(19) United States
(12) Patent Application Publication
(10) Pub. No.: US 2026/0206487 A1
(43) Pub. Date: Jul. 16, 2026

(54) CONDENSED RING COMPOUND, LIGHT-EMITTING DEVICE, AND ELECTRONIC

(71) Applicant: Samsung Display Co., LTD., Yongin-si (KR)

(72) Inventors: Ha Young YU, Yongin-si (KR); Gyeong Heon KIM, Yongin-si (KR); Hoi Lim KIM, Yongin-si (KR)

(21) Appl. No.: 19/387,834

(22) Filed: Nov. 13, 2025

(30) Foreign Application Priority Data
Jan. 10, 2025 (KR) 10-2025-0004185

(51) Int. Cl. H10K 85/60; C07D 491/048; C07D 495/04; H10K 50/15; H10K 50/17; H10K 50/18

(52) U.S. Cl. CPC H10K 85/6574; C07D 491/048; C07D 495/04; H10K 85/654; H10K 85/6572; H10K 85/6576; H10K 50/15; H10K 50/17; H10K 50/181

(57) ABSTRACT

Provided are a condensed ring compound represented by Chemical Formula 1, a light-emitting device that includes the condensed ring compound, and an electronic device that includes the light-emitting device. The light-emitting device includes a first electrode, a second electrode, and an intermediate layer disposed between the first electrode and the second electrode. The intermediate layer includes a hole transfer region, an emission layer, and an electron transfer region, and the hole transfer region includes the condensed ring compound represented by Formula 1.

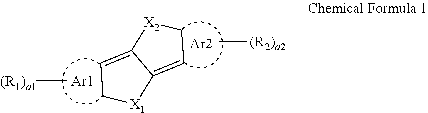

wherein Chemical Formula 1 is as defined herein.

### CROSS-REFERENCE TO RELATED APPLICATION

This application claims priority to and the benefit of Korean Patent Application No. 10-2025-0004185, filed on Jan. 10, 2025 in the Korean Intellectual Property Office (KIPO), the entire content of which is incorporated by reference herein.

### TECHNICAL FIELD

Embodiments of the present disclosure relate to a condensed ring compound, a light-emitting device, and an electronic device.

### BACKGROUND

An organic light-emitting diode (OLED) display has a self-luminous property, and may provide improved viewing angle and contrast properties. Additionally, a high response rate and a high luminance may be provided.

A light-emitting device may include an emission layer disposed between a first electrode and a second electrode. A hole transferred from the first electrode and an electron transferred from the second electrode may be recombined in the emission layer to generate an exciton. Light emission properties are implemented as the exciton is shifted from an excited state to a ground state.

### SUMMARY

According to an aspect, there is provided a condensed ring compound having improved driving voltage, luminous efficiency, and life-span property.

According to an aspect, there is provided a light-emitting device having improved driving voltage, luminous efficiency, and life-span property.

According to an aspect, there is provided an electronic device having driving voltage, luminous efficiency, and life-span property.

The condensed ring compound is represented by Chemical Formula 1.

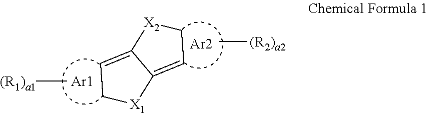

In Chemical Formula 1, X₁ and X₂ are each independently oxygen, sulfur, or selenium; and Ar₁ and Ar₂ are each independently a fused C₆-C₆₀ ring including an aromatic ring.

In Chemical Formula 1, R₁ and R₂ are each independently hydrogen, deuterium, —OH, —CN, —F, —Cl, —Br, —I, —SF₅, —CD₃, —CD₂H, —CDH₂, —CF₃, —CF₂H, —CFH₂, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₁-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₁-C₆₀ heteroaryloxy group, a substituted or unsubstituted C₁-C₆₀ heteroarylthio group, a substituted or unsubstituted C₈-C₆₀ condensed polycyclic group, or a substituted or unsubstituted silyl group. The silyl group may be as defined herein.

In Chemical Formula 1, at least one of R₁ and R₂ is an electron-withdrawing group or a group substituted with an electron-withdrawing group, wherein the electron-withdrawing group has a para Hammett substituent constant (σₚ) of greater than 0 according to the Hammett equation.

In Chemical Formula 1, a1 and a2 are each independently an integer of 1 to 6.

In Chemical Formula 1, when R₁ and R₂ are each independently 2 or more, two or more of each of R₁ and R₂ are the same or different.

In Chemical Formula 1, two or more of adjacent R₁ are optionally combined with each other to form a saturated ring or an unsaturated ring, and two or more of adjacent R₂ are optionally combined with each other to form a saturated ring or an unsaturated ring.

In some embodiments, the electron-withdrawing group may each independently be —F, —Cl, —Br, —I, —SF₅, —CF₃, —CN, —SCN, —SOCH₃, —SOCH₂CH₃, —SCH(CH₃)₂, —NO₂, or a □ electron-depleted nitrogen-containing C₃-C₃₀ cyclic group.

A light-emitting device may include a first electrode, a second electrode, and an intermediate layer disposed between the first electrode and the second electrode. The intermediate layer may include a hole transfer region, an emission layer, and an electron transfer region, and the hole transfer region may include the condensed ring compound represented by Chemical Formula 1.

An electronic device may include the light-emitting device.

The condensed ring compound represented by Chemical Formula 1 according to embodiments of the present disclosure may provide improved driving voltage, luminous efficiency, and life-span property.

The light-emitting device according to embodiments of the present disclosure and the electronic device including the light-emitting device may provide improved driving voltage, luminous efficiency, and life-span property.

### BRIEF DESCRIPTION OF THE DRAWINGS

FIGS. 1 to 6 are schematic cross-sectional views illustrating light-emitting devices in accordance with one or more exemplary embodiments.

FIG. 7 is a schematic cross-sectional view illustrating a display device in accordance with one or more exemplary embodiments.

FIG. 8 is a schematic cross-sectional view illustrating a display device in accordance with one or more exemplary embodiments.

FIG. 9 is a schematic cross-sectional view illustrating a stack construction of light-emitting structure in a display device in accordance with one or more exemplary embodiments.

FIG. 10 is a schematic cross-sectional view illustrating a display device in accordance with one or more exemplary embodiments.

FIG. 11 is a schematic cross-sectional view illustrating a display device in accordance with one or more exemplary embodiments.

FIG. 12 is a schematic exploded perspective view illustrating an electronic device in accordance with one or more exemplary embodiments.

FIG. 13 is a schematic view illustrating an electronic device in accordance with one or more exemplary embodiments.

FIG. 14 is a block diagram of an electronic device in accordance with one or more exemplary embodiments.

FIG. 15 are schematic diagrams of electronic devices in accordance with one or more exemplary embodiments.

### DETAILED DESCRIPTION

A condensed ring compound of Chemical Formula 1 includes a core including a dihydrofuran structure, and two or more aromatic ring structures fused to the core, and has one or more electron-withdrawing groups or one or more groups substituted with an electron-withdrawing group bonded to the aromatic ring structure. Accordingly, energy levels of HOMO and LUMO may be adjusted to levels appropriate for exciton formation in an emission layer. Additionally, the condensed ring compound may provide improved hole mobility and lower electron mobility.

The condensed ring compound of Chemical Formula 1 has an increased conjugation length to have improved structural and chemical stability.

In example embodiments, the condensed ring compound of Chemical Formula 1 may be used in a hole transfer region of a light-emitting device to improve efficiency and life-span properties of a light-emitting device.

According to the present disclosure, a light-emitting device and an electronic device including the condensed ring compound of Chemical Formula 1 are also provided.

### Definition of Terminology

The present disclosure may have various modifications and may be embodied in different forms, and example embodiments will be explained in more detail with reference to the accompany drawings. The present disclosure may, however, be embodied in different forms and should not be construed as being limited to the embodiments set forth herein. Rather, all modifications, equivalents, and substituents that are included in the spirit and technical scope of the present disclosure should be included herein.

Like reference numerals refer to like elements throughout, and duplicative descriptions thereof may not be provided. In the drawings, the dimensions of structures may be exaggerated for clarity of illustration. It will be understood that, although the terms first, second, etc. may be used herein to describe various elements, these elements should not be limited by these terms. These terms are only used to distinguish one element from another element. Thus, a first element could be alternatively termed a second element without departing from the teachings of the present disclosure. Similarly, a second element could be termed a first element. As used herein, singular forms such as “a,” “an,” and “the” are intended to include the plural forms as well, unless the context clearly indicates otherwise.

It will be further understood that the terms “includes,” “including,” “comprises,” and/or “comprising,” when used in this specification, specify the presence of stated features, numerals, steps, operations, elements, parts, or the combination thereof, but do not preclude the presence or addition of one or more other features, numerals, steps, operations, elements, parts, or the combination thereof.

As used herein, expressions such as “at least one of”, “one of”, and “selected from”, when preceding a list of elements, modify the entire list of elements and do not modify the individual elements of the list. As used herein, the term “and/or” includes any and all combinations of one or more of the associated listed items. Further, the use of “may” when describing embodiments of the present disclosure refers to “one or more embodiments of the present disclosure”.

It will also be understood that when a layer, a film, a region, a plate, etc. is referred to as being “on” or “above” another part, it can be “directly on” the other part, or intervening layers may also be present. When a layer, a film, a region, a plate, etc. is referred to as being “under” or “below” another part, it can be “directly under” the other part, or intervening layers may also be present. Also, when an element is referred to as being disposed “on” another element, it can be disposed under the other element.

As used herein, the term “substituted or unsubstituted” may refer to being substituted or unsubstituted by one or more substituents that are, for example, a deuterium atom, a halogen atom, a cyano group, a nitro group, an amino group, a silyl group, an oxy group, a thio group, a sulfinyl group, a sulfonyl group, a carbonyl group, an ester group, boron, a phosphine oxide group, a phosphine sulfide group, an alkyl group (e.g., a C₁-C₆₀ alkyl group, C₁-C₁₀ alkyl group), an alkenyl group (e.g., a C₂-C₆₀ alkenyl group, C₂-C₁₀ alkenyl group), an alkynyl group (e.g., a C₂-C₆₀ alkynyl group, C₂-C₁₀ alkynyl group), an alkoxy group (e.g., a C₁-C₆₀ alkoxy group, C₁-C₁₀ alkoxy group), a hydrocarbon ring group, an aryl group (e.g., a C₆-C₆₀ aryl group), or a heterocyclic group (e.g., a C₁-C₆₀ heterocyclic group). For example, the term “substituted alkyl group” may refer to a group in which at least one of hydrogen atoms of the alkyl group is substituted with the above-described substituent, and thus the substituent is further bonded to a carbon atom of the alkyl group.

The substituent may include a combination of substituents selected from the groups described above. For example, at least one hydrogen atom in the alkyl group, the aryl group, etc., included as a substituent may be substituted with a deuterium atom, a halogen atom, a cyano group, a nitro group, an amino group, a silyl group, an oxy group, a thio group, a sulfinyl group, a sulfonyl group, a carbonyl group, an ester group, boron, a phosphine oxide group, a phosphine sulfide group, an alkyl group, an alkenyl group, an alkynyl group, a hydrocarbon ring group, an aryl group, a heterocyclic group, or a combination thereof.

In the substituents described above, a multivalent substituent such as an amino group, a phosphine sulfide group, a phosphine oxide group, a sulfinyl group, a sulfonyl group, an oxy group, a carbonyl group, an ester group, etc., may each independently be substituted with a C₁-C₁₀ alkyl group, a C₁-C₁₀ alkenyl group, a C₁-C₁₀ alkynyl group, or a C₆-C₁₀ aryl group.

As used herein, the term “substituted or unsubstituted Cₐ-C_{b} Y group” the range of a to b refers to the number of carbon atoms in an unsubstituted Y group, and may not include the number of carbon atoms of a substituent group that is bonded thereto.

As used herein, the term “alkyl group” may be a monovalent hydrocarbon group in which one hydrogen atom is removed from a linear or branched hydrocarbon group. Non-limiting examples of an alkyl group may include a methyl group, an ethyl group, a propyl group, a sec-butyl group, a tert-butyl group, an iso-butyl group, a pentyl group, a neopentyl group, a 2-ethyl butyl group, a 3,3-dimethyl butyl group, a hexyl group, a heptyl group, an octyl group, or the like.

As used herein, the term “alkylene group” may be a divalent hydrocarbon group in which two hydrogen atoms are removed from a linear or branched hydrocarbon group.

As used herein, the term “alkenyl group” may have the same skeleton as that of an alkyl group, and may be a monovalent hydrocarbon group that includes at least one carbon-carbon double bond. As used herein, the term “alkenylene group” may be a divalent hydrocarbon group in which one hydrogen atom is further removed from an alkenyl group.

As used herein, the term “alkynyl group” may have the same skeleton as that of an alkyl group, and may be a monovalent hydrocarbon group that includes at least one carbon-carbon triple bond. As used herein, the term “alkynylene group” may be a divalent hydrocarbon group in which one hydrogen atom is further removed from an alkynyl group.

As used herein, the term “aryl group” may be a monovalent hydrocarbon group in which one hydrogen atom is removed from a hydrocarbon group having an aromatic structure. The definition of an aryl group may also encompass a group in which multiple aromatic rings are directly connected, such as a biphenyl group. Non-limiting examples of an aryl group may include, e.g., a phenyl group, a naphthyl group, an anthracenyl group, a phenanthrenyl group, a pyrenyl group, a fluorenyl group, a tetracenyl group, a biphenyl group, a terphenyl group, a quaterphenyl group, a chrysenyl group, or the like.

As used herein, a group in which two or more aryl rings are condensed to each other or linked to each other by an alicyclic hydrocarbon ring, such as a fluorenyl group, can be encompassed in the definition of an aryl group.

For example, a biphenyl group may be interpreted as an aryl group, or may be interpreted as a phenyl group that is substituted with a phenyl group.

As used herein, the term “arylene group” may be a divalent hydrocarbon group in which two hydrogen atoms are removed from an aryl group.

As used herein, the term “heteroaryl group” may be a monovalent group having an aromatic structure that includes at least one heteroatom selected from B, O, N, P, S, Si, Se, or Ge as a ring-forming atom. As used herein, the term “heteroarylene group” may be a divalent group having an aromatic structure that includes at least one heteroatom such as B, O, N, P, S, Si, Se, or Ge as a ring-forming atom. When a heteroaryl group or a heteroarylene group includes two or more heteroatoms, the two or more heteroatoms may be the same as or different from each other. Non-limiting examples of the heteroaryl group include thiophene, furan, pyrrole, imidazole, thiazole, oxazole, oxadiazole, triazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzimidazole, benzothiazole, benzocarbazole, benzothiophene, dibenzothiophene, thienothiophene, benzofuran, phenanthroline, isooxazole, oxadiazole, thiadiazole, phenothiazine, dibenzosilole, dibenzofuran, or the like.

As used herein, a group in which two or more aryl rings are condensed or linked to a non-aromatic heterocyclic ring, such as a carbazole group, can also be encompassed in the definition of a heteroaryl group.

As used herein, the term “cyclic group” may encompass a monocyclic group or a polycyclic group, and may also encompass an alicyclic ring or an aromatic ring.

As used herein, the term “polycyclic group” may be a group in which two or more rings are connected to each other or condensed to each other through one or more atoms. For example, a polycyclic structure may include a bicyclic structure through a bridge carbon, a spiro structure, a fused structure, or the like.

As used herein, the term “condensed group” or “condensed ring structure” may each be a group in which two or more adjacent rings share two or more atoms among the above-described polycyclic structures. Non-limiting examples of a condensed ring structure may include naphthalene, anthracene, phenanthrene, fluorene, pyrene, benzopyrene, pentacene, polyacene, helicene, or the like.

As used herein, the term “carbocyclic group (e.g., C₃-C₆₀ carbocyclic group)” may be a cyclic group in which carbon atoms are the only ring-forming atoms. As used herein, the term “heterocyclic group” (e.g., a C₁-C₆₀ heterocyclic group) may be a cyclic group that includes at least one heteroatom as a ring-forming atom, in addition to carbon atoms as ring-forming atoms.

As used herein, a carbocyclic group and a heterocyclic group may each independently be a monocyclic group that consists of one ring or a polycyclic group in which two or more rings are condensed with each other.

As used herein, the term “hydrocarbon ring” may refer to an optional functional group or substituent derived from an aliphatic hydrocarbon ring. The hydrocarbon ring group may be a saturated hydrocarbon ring group of 5 to 30 carbon atoms for forming a ring.

As used herein, the term “silyl group” includes an alkyl silyl group and an aryl silyl group. Non-limiting examples of the silyl group include trimethylsilyl, triethylsilyl, t-butyldimethylsilyl, vinyldimethylsilyl, propyldimethylsilyl, triphenylsilyl, diphenylsilyl, phenylsilyl, or the like. However, an embodiment of the present disclosure is not limited thereto.

As used herein, the carbon number of the amino group is not specifically limited, but may be 1 to 30. The amino group may include an alkyl amino group, an aryl amino group, or a heteroaryl amino group. Non-limiting examples of the amino group include a methylamino group, a dimethylamino group, a phenylamino group, a diphenylamino group, a naphthylamino group, a 9-methyl-anthracenylamino group, a triphenylamino group, or the like.

### Condensed Ring Compound

The condensed ring compound is represented by Chemical Formula 1.

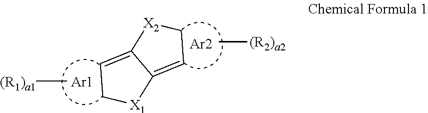

In Chemical Formula 1, X₁ and X₂ are each independently oxygen, sulfur, or selenium. Ar₁ and Ar₂ are each independently a fused C₆-C₆₀ ring including an aromatic ring.

In Chemical Formula 1, R₁ and R₂ are each independently hydrogen, deuterium, —OH, —CN, —F, —Cl, —Br, —I, —SF₅, —CD₃, —CD₂H, —CDH₂, —CF₃, —CF₂H, —CFH₂, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₁-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₁-C₆₀ heteroaryloxy group, a substituted or unsubstituted C₁-C₆₀ heteroarylthio group, a substituted or unsubstituted C₈-C₆₀ condensed polycyclic group, or a substituted or unsubstituted silyl group.

In Chemical Formula 1, at least one of R₁ and R₂ is an electron-withdrawing group or a group substituted with an electron-withdrawing group. The electron-withdrawing group is a group having a para Hammett substituent constant (σₚ) of greater than 0 according to the Hammett equation.

In Chemical Formula 1, a1 and a2 are each independently an integer of 1 to 6.

In Chemical Formula 1, when R₁ and R₂ are each independently 2 or more, two or more of each of R₁ and R₂ are the same or different.

In Chemical Formula 1, two or more of adjacent R₁ are optionally combined with each other to form a saturated ring or an unsaturated ring, and two or more of adjacent R₂ are optionally combined with each other to form a saturated ring or an unsaturated ring.

The condensed ring compound represented by Chemical Formula 1 may have high structural and chemical stability by an extended conjugation length, and may easily generate excitons by improving hole mobility and reducing electron mobility.

A light-emitting device including the condensed ring compound represented by Formula 1 may provide sufficient luminescence efficiency and enhanced high life-span at a relatively low driving voltage.

In some embodiments, the saturated ring may be selected from a 5-membered ring, a 6-membered ring, and a 7-membered ring, and the above rings may be a hydrocarbon ring or a heteroatom-containing ring. The saturated ring may be unsubstituted or substituted with at least one of deuterium, —F, —Cl, —CD₃, —CD₂H, —CDH₂, a C₁-C₁₀ straight-chain alkyl group, a C₃-C₁₀ branched alkyl group, a C₂-C₁₀ straight-chain alkenyl group, a C₃-C₁₀ branched alkenyl group, or a C₆-C₁₀ aryl group.

In some embodiments, the unsaturated ring can be independently selected from a 5-membered ring, a 6-membered ring, and a 7-membered ring, and the above ring may be a hydrocarbon ring or a heteroatom-containing ring. The unsaturated ring may be, e.g., a cycloalkene or an aromatic ring containing a C═C unsaturated double bond. The unsaturated ring may be unsubstituted or substituted with at least one of deuterium, —F, —Cl, —CD₃, —CD₂H, —CDH₂, a C₁-C₁₀ straight-chain alkyl group, a C₃-C₁₀ branched alkyl group, a C₂-C₁₀ straight-chain alkenyl group, a C₃-C₁₀ branched alkenyl group, or a C₆-C₁₀ aryl group.

The condensed ring compound represented by Formula 1 includes at least one electron-withdrawing group to improve hole mobility.

The Hammett equation may be used as a measure of a degree of electron-withdrawing or electron-donating of a functional group. The para substituent constant (σₚ) determined by the Hammett equation can be found in the literature such as “A survey of Hammett substituent constants and resonance and field parameters,” but even without reference to such literature, the para substituent constant (σₚ) can be measured based on the Hammett equation.

In some embodiments, the electron withdrawing group may have a para substituent constant (σₚ) of greater than 0.05 according to the Hammett equation.

In some embodiments, the electron withdrawing group may be —F, —Cl, —Br, —I, —SF₅, —CF₃, —CN, —SCN, —SOCH₃, —SOCH₂CH₃, —SCH(CH₃)₂, —NO₂, or a π electron-depleted nitrogen-containing C₃-C₃₀ cyclic group. Accordingly, the HOMO and LUMO energy levels of the condensed ring compound represented by Formula 1 may be adjusted to further enhance hole mobility and to further reduce electron mobility.

In some embodiments, the electron withdrawing group may be independently —F, —Cl, —Br, —I, —SF₅, —CF₃, —CN, —SCN, —SOCH₃, —SOCH₂CH₃, —SCH(CH₃)₂, —NO₂, a substituted or unsubstituted triazine group, a substituted or unsubstituted thiazole group, a substituted or unsubstituted benzothiazole group, a substituted or unsubstituted pyrazine group, a substituted or unsubstituted pyridine group, a substituted or unsubstituted pyridazine group, a substituted or unsubstituted pyrimidine group, a substituted or unsubstituted naphthyridine group, a substituted or unsubstituted phthalazine group, a substituted or unsubstituted quinoline group, a substituted or unsubstituted quinazoline group, a substituted or unsubstituted benzocinnoline group, a substituted or unsubstituted phenanthroline group, and a substituted or unsubstituted acridine group.

In some embodiments, the substitution may further include substitution with the electron withdrawing group. For example, the substituted triazine group, the substituted thiazole group, the substituted benzothiazole group, the substituted pyrazine group, the substituted pyridine group, the substituted pyridazine group, the substituted pyrimidine group, the substituted naphthyridine group, the substituted phthalazine group, the substituted quinoline group, the substituted quinazoline group, the substituted benzocinnoline group, the substituted phenanthroline group, and the substituted acridine group may be a triazine group, a thiazole group, a benzothiazole group, a pyrazine group, a pyridine group, a pyridazine group, a pyrimidine group, a naphthyridine group, a phthalazine group, a quinoline group, a quinazoline group, a benzocinnoline group, a phenanthroline group, and an acridine group, respectively, may be substituted with at least one of —F, —Cl, —Br, —I, —SF₅, —CF₃, —CN, —SCN, —SOCH₃, —SOCH₂CH₃, —SCH(CH₃)₂, —NO₂, a triazine group, a thiazole group, a benzothiazole group, a pyrazine group, a pyridine group, a pyridazine group, a pyrimidine group, a naphthyridine group, a phthalazine group, a quinoline group, a quinazoline group, a benzocinnoline group, a phenanthroline group, or an acridine group.

In some embodiments, Ar₁ and Ar₂ may each independently represent a group in which a benzene ring, a naphthalene ring, an anthracene ring, a phenanthrene ring, a tetracene ring, a pyrene ring, or a benzopyrene ring is fused.

In some embodiments, Ar₁ and Ar₂ may each independently represent a group in which a benzene ring or a naphthalene ring is fused.

Accordingly, the condensed ring compound represented by Chemical Formula 1 may have an extended conjugation length, thereby further improving structural and chemical stability.

In some embodiments, the condensed ring compound of Chemical Formula 1 may be represented by Chemical Formula 1-1.

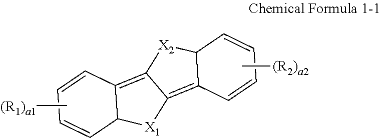

wherein, in Chemical Formula 1-1, a1 and a2 are each independently an integer of 1 to 4. The definitions of X₁, X₂, R₁, and R₂ as the same as provided for Chemical Formula 1.

In some embodiments, the condensed ring compound of Chemical Formula 1 may be represented by Chemical Formula 1-2, Chemical Formula 1-3, or Chemical Formula 1-4.

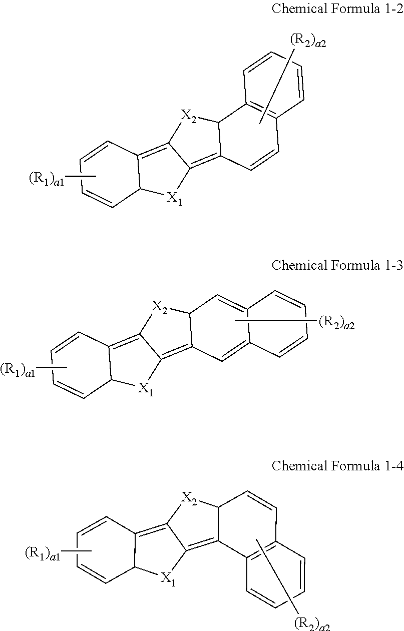

wherein, in Chemical Formulae 1-2, 1-3, and 1-4, a1 is an integer of 1 to 4, and a2 is an integer of 1 to 6. The definitions of X₁, X₂, R₁, and R₂ as the same as provided for Chemical Formula 1.

In some embodiments, the condensed ring compound of Chemical Formula 1 may be represented by Chemical Formula 1-5, Chemical Formula 1-6, Chemical Formula 1-7, Chemical Formula 1-8, Chemical Formula 1-9, or Chemical Formula 1-10.

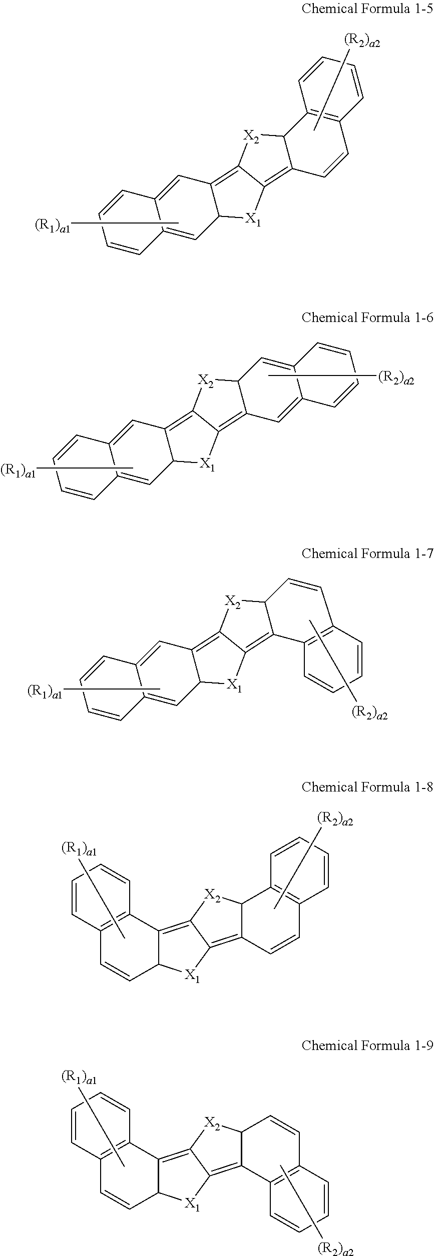

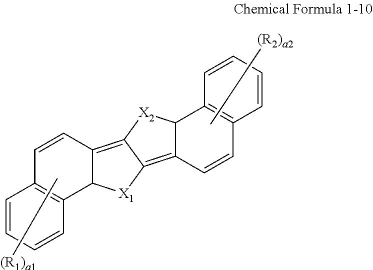

In some embodiments, the condensed ring compound represented by Chemical Formula 1 may have a hole mobility from 1.30×10⁻³ centimeter squared per volt second (cm²/Vs) to 2.50×10⁻² cm²/Vs. In an embodiment, the hole mobility of the condensed ring compound represented by Chemical Formula 1 may be from 1.35×10⁻³ cm²/Vs to 2.30×10⁻³ cm²/Vs. The hole mobility may be determined by a space-charge-limited current (SCLC) method.

In some embodiments, the condensed ring compound represented by Chemical Formula 1 may have an electron mobility from 4.00×10⁻³ cm²/Vs to 3.00×10⁻² cm²/Vs. In an embodiment, the electron mobility of the condensed ring compound represented by Chemical Formula 1 may be from 4.10×10⁻³ cm²/Vs to 2.80×10⁻² cm²/Vs. The electron mobility may be determined by a space-charge-limited current (SCLC) method.

In the above-described ranges of the hole mobility and the electron mobility, the condensed ring compound represented by Chemical Formula 1 may generate excitons more efficiently. In some embodiments, the condensed ring compound represented by Chemical Formula 1 may have a glass transition temperature from 110° C. to 190° C. In an embodiment, the glass transition temperature of the condensed ring compound represented by Chemical Formula 1 may be from 120° C. to 180° C. In the above-described ranges of the glass transition temperature, the life-span properties may be further improved. The glass transition temperature may be determined by differential scanning calorimetry (DSC).

In some embodiments, the condensed ring compound represented by Chemical Formula 1 may have a highest occupied molecular orbital (HOMO) energy level from −9.0 electron volts (eV) to −6.0 eV. In an embodiment, the HOMO energy level of the condensed ring compound represented by Chemical Formula 1 may be from −8.5 eV to −6.2 eV. The HOMO energy level may be determined by cyclic voltammetry (CV).

In some embodiments, the condensed ring compound represented by Chemical Formula 1 may have a lowest unoccupied molecular orbital (LUMO) energy level from −5.8 eV to −4.9 eV. In an embodiment, the LUMO energy level of the condensed ring compound represented by Chemical Formula 1 may be from −5.6 eV to −5.0 eV. The LUMO energy level may be determined by cyclic voltammetry (CV).

The condensed ring compound represented by Chemical Formula 1 may have the HOMO energy level and/or LUMO energy level in the above-described ranges, so that the light-emitting device including the condensed ring compound represented by Chemical Formula 1 may have improved efficiency and life-span properties at a relatively low driving voltage.

In some embodiments, the condensed ring compound represented by Chemical Formula 1 may be one of the compounds 1 to 36:

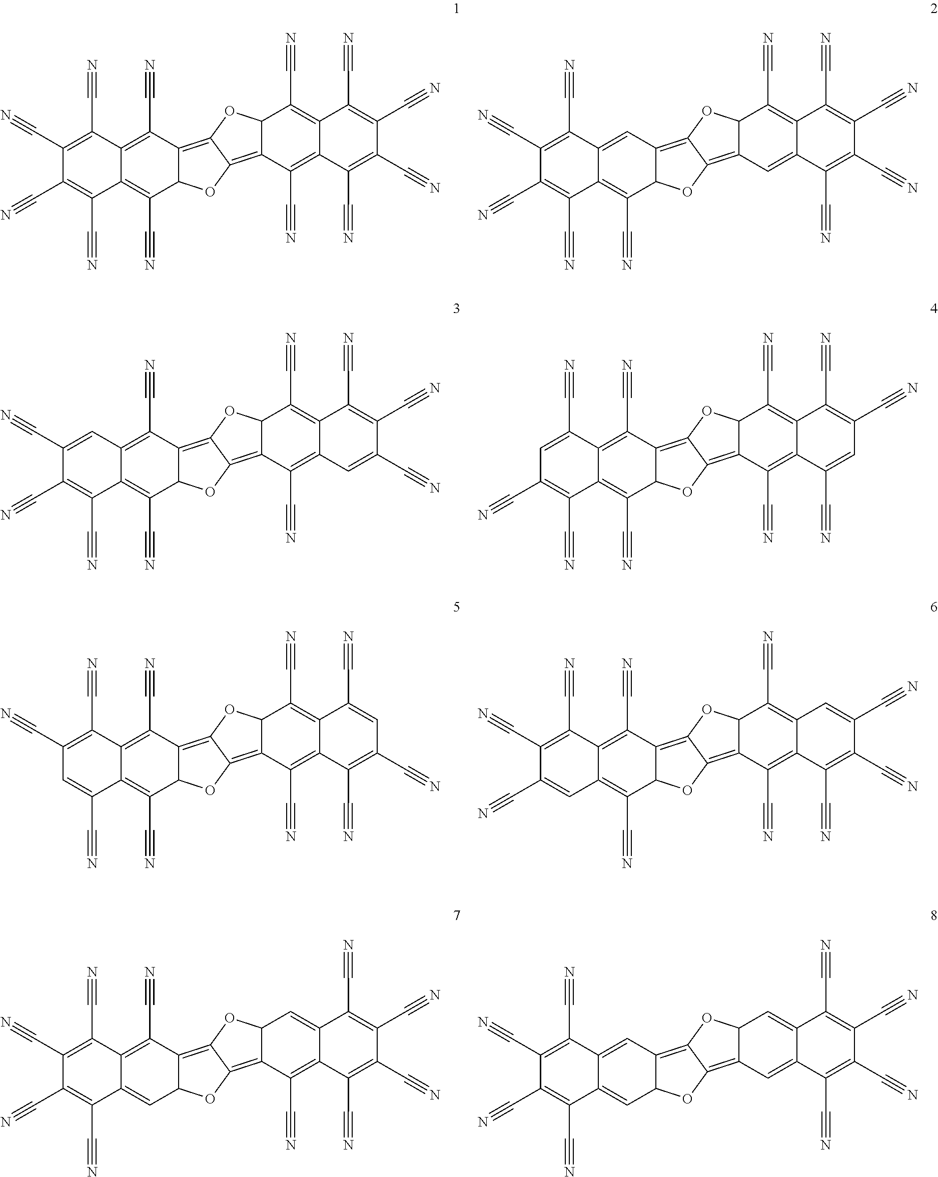

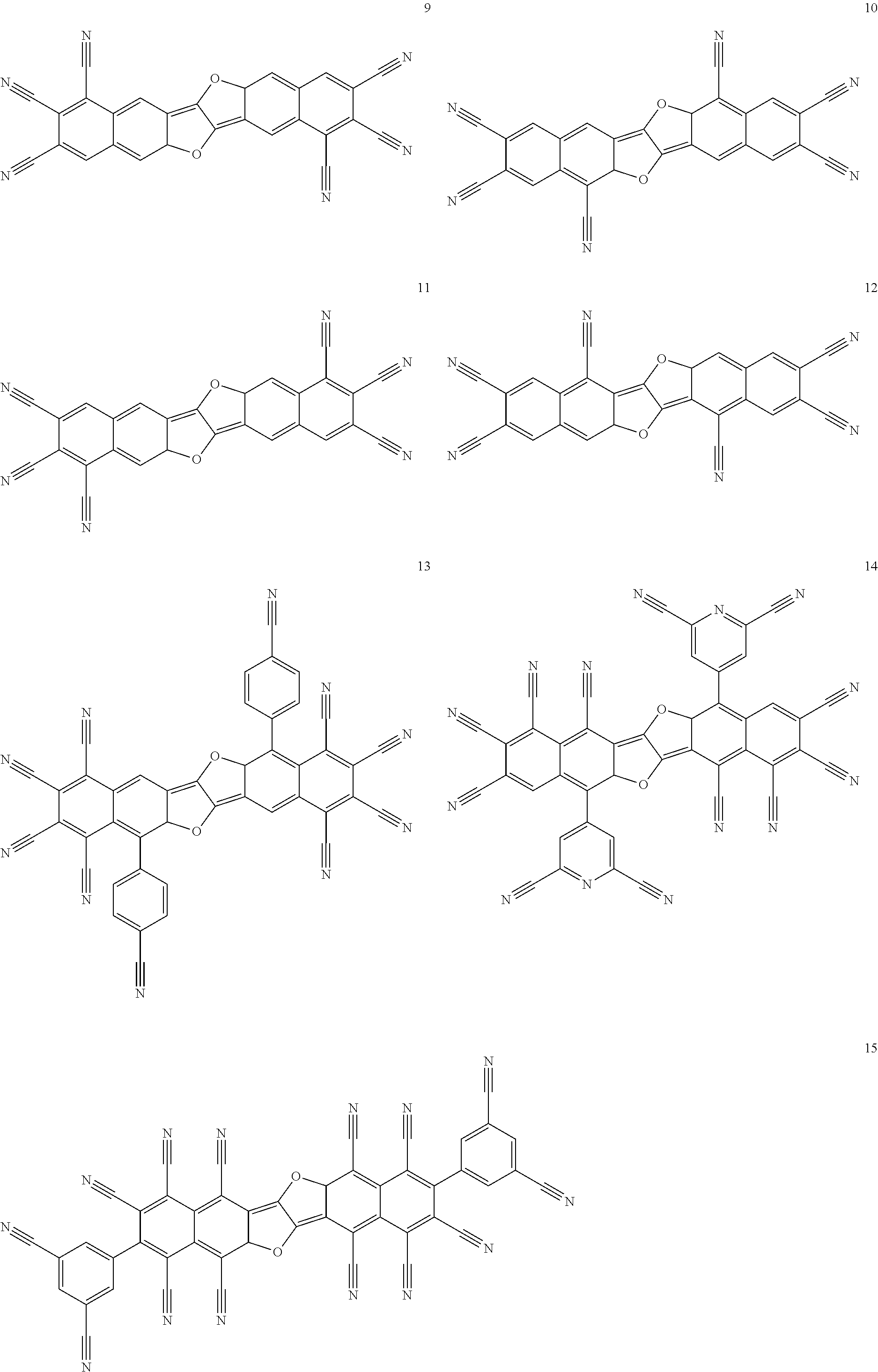

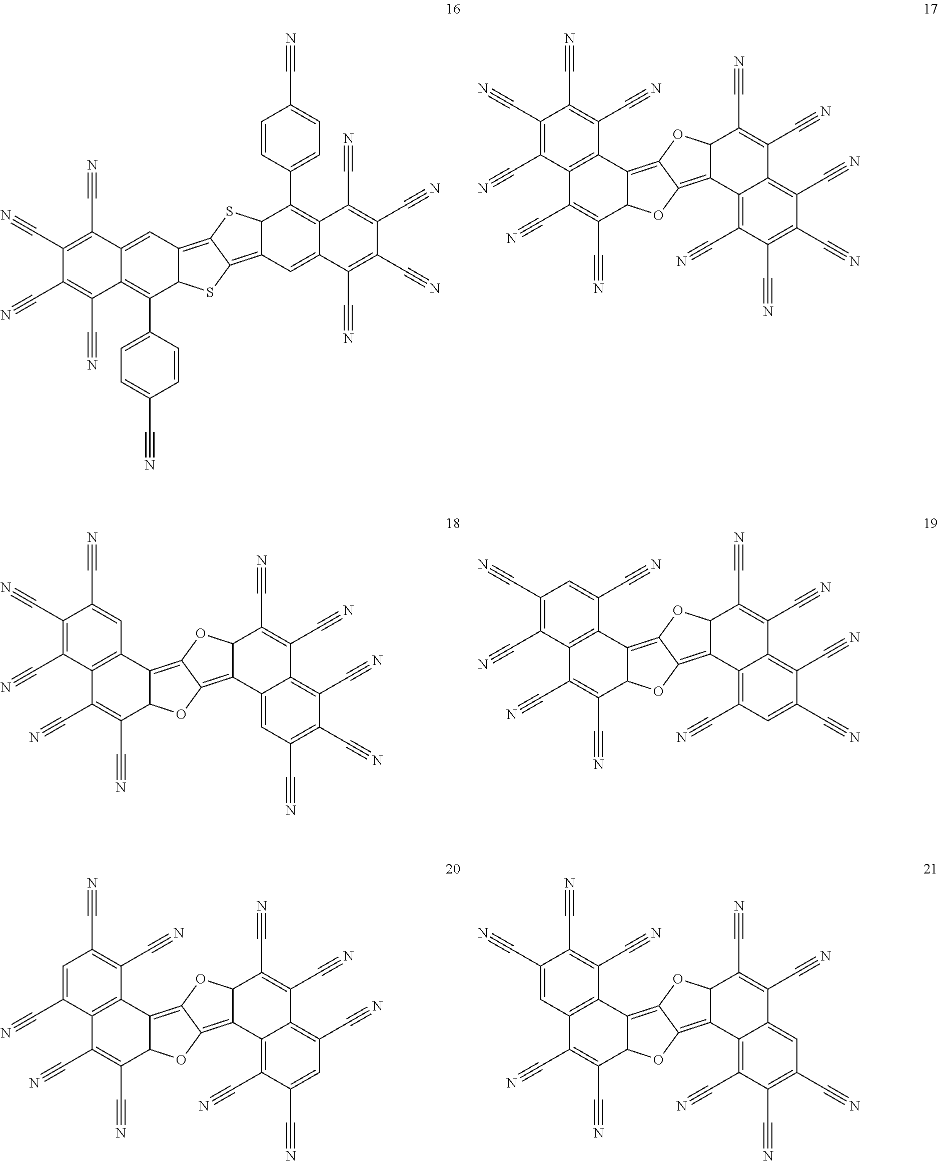

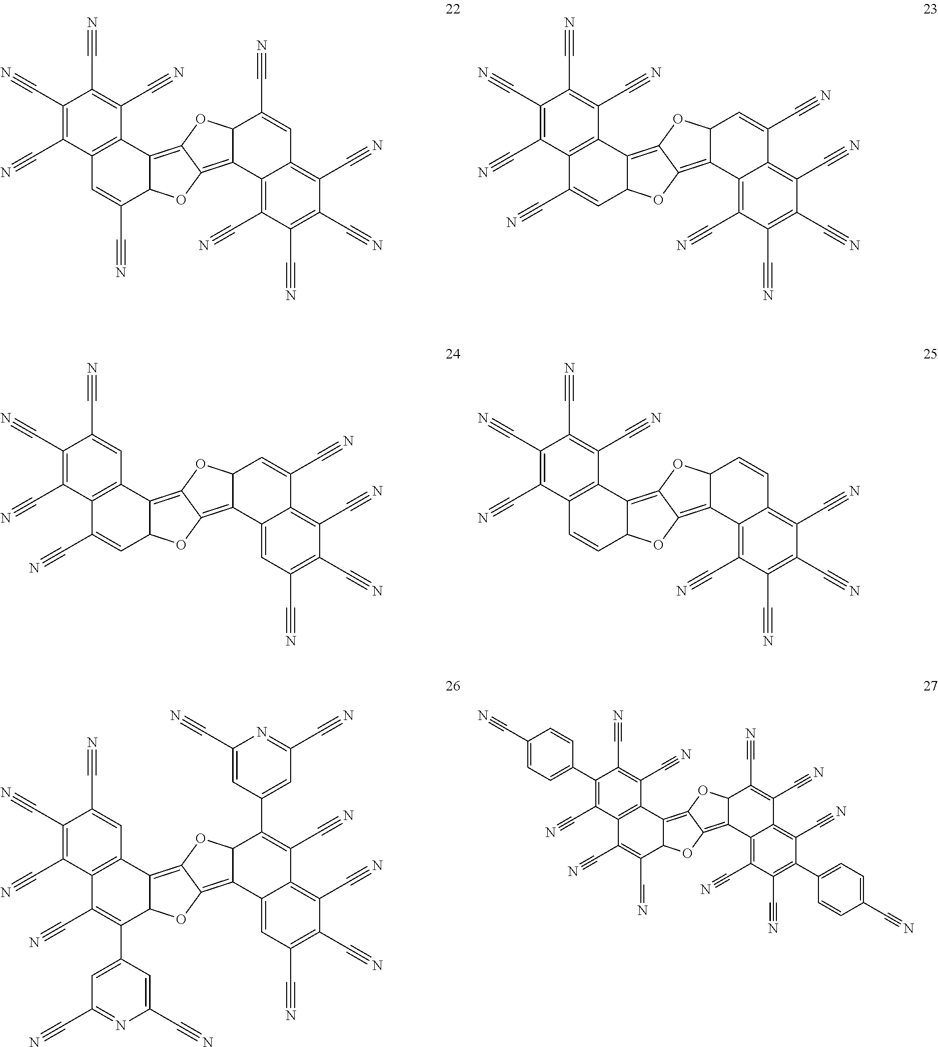

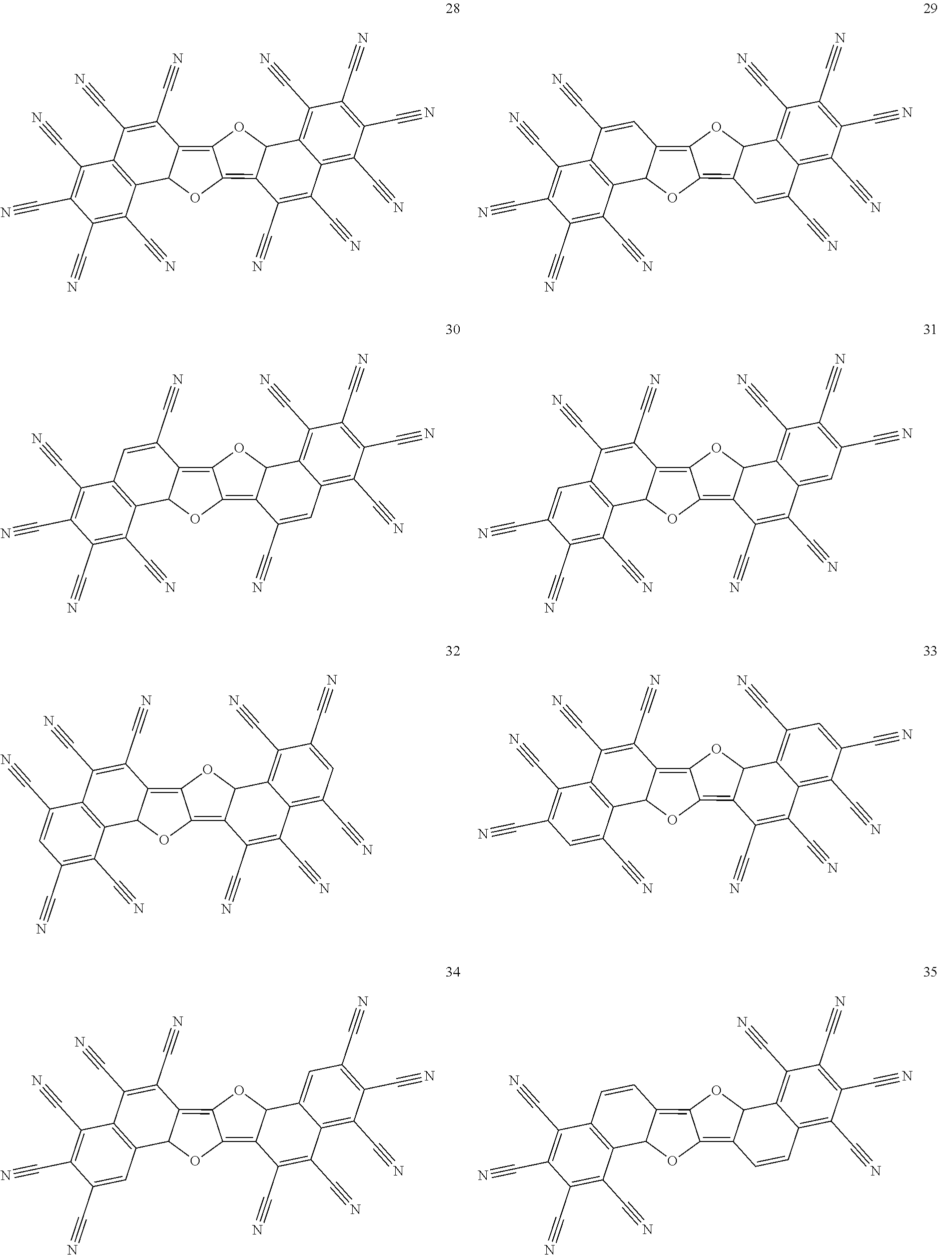

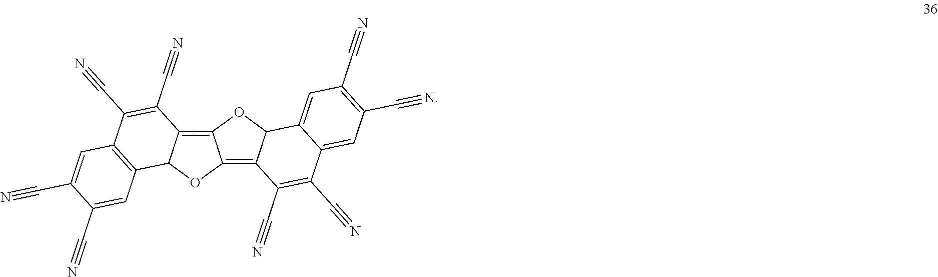 [SMILES: N#CC1=C(C#N)c2cc(C#N)c(C#N)cc2C2OC3=C4C(C#N)=C(C#N)c5cc(C#N)c(C#N)cc5C4OC3=C12]

### Light-Emitting Device

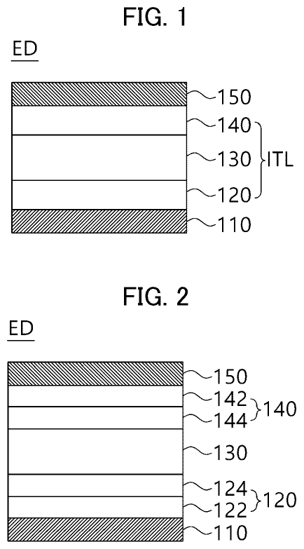

FIGS. 1 to 6 are schematic cross-sectional views illustrating light-emitting devices in accordance with example embodiments.

Referring to FIG. 1, a light-emitting device ED may include a first electrode 110, a second electrode 150, and an intermediate layer ITL disposed between the first electrode 110 and the second electrode 150. The intermediate layer ITL may include a hole transfer region 120, an emission layer 130, and an electron transfer region 140.

The hole transfer region 120 may include the above-described condensed ring compound represented by Chemical Formula 1 to achieve improved driving voltage and life-span properties, and may provide sufficient luminous efficiency.

In some embodiments, the hole transfer region 120 may include at least one of a hole injection layer 122, a hole transport layer 124, or an electron blocking layer 126, and at least one of the hole injection layer 122, the hole transport layer 124, or the electron blocking layer 126 may include the condensed ring compound represented by Chemical Formula 1.

In some embodiments, at least one of the hole injection layer 122, the hole transport layer 124, or the electron blocking layer 126 may include at least one of the above-described condensed ring compounds represented by Chemical Formula 1.

In some embodiments, the condensed ring compound represented by Chemical Formula 1 may include at least one compound represented by any one of Chemical Formulae 1-1 to 1-8 described herein.

In some embodiments, the condensed ring compound represented by Chemical Formula 1 may include at least one of the compounds 1 to 36.

In some embodiments, the emission layer 130 may emit a blue light having a maximum emission central wavelength from 430 nanometers (nm) to 490 nm.

The light-emitting device ED may include, e.g., two or more light-emitting structures each of which included the emission layer 130 between the first electrode 110 and the second electrode 150. The light-emitting structure may include, e.g., a stacked structure of the hole transfer region 120, the emission layer 130, and the electron transfer region 140. A charge generation layer may be disposed between the light-emitting structures. The charge generation layer may include a p-type charge generation layer and/or an n-type charge generation layer.

The first electrode 110 may be an anode or a cathode. In some embodiments, the first electrode 110 may serve as an anode, and may serve as a pixel electrode. In this case, the first electrode 110 may include a conductive material with a high work function that promotes hole injection.

In an embodiment, the first electrode 110 may be a transmissive electrode. The first electrode 110 may include a transparent conductive oxide such as indium tin oxide (ITO), indium zinc oxide (IZO), zinc oxide (ZnO), indium tin zinc oxide (ITZO), or the like.

In an embodiment, the first electrode 110 may be a translucent electrode or a reflective electrode. The first electrode 110 may include at least one of Ag, Mg, Cu, Al, Pt, Pd, Au, Ni, Nd, Ir, Cr, Li, Ca, Mo, Ti, W, In, Sn, Zn, or an alloy or a compound (e.g., LiF) containing at least one therefrom. For example, the first electrode 110 may include Li, Ca, LiF/Ca (a stacked structure of LiF and Ca), LiF/Al (a stacked structure of LiF and Al), a mixture of Ag and Mg, or the like, but embodiments are not limited thereto.

The first electrode 110 may have a single-layered structure or a multi-layered structure. For example, the first electrode 110 may have a triple-layered structure of ITO/Ag/ITO.

A thickness of the first electrode 110 may be about 700 angstroms (Å) to about 10,000 Å. For example, the thickness of the first electrode 110 may be about 1,000 Å to about 3,000 Å.

The second electrode 150 may be a cathode or an anode. In some embodiments, the second electrode 150 may serve as an electron injection electrode or as a cathode. The second electrode 150 may include a metal, an alloy, an electrically conductive compound, or the like, each having a low work function.

For example, the second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or the like. The second electrode 150 may include one of the aforementioned materials, or a combination thereof.

The second electrode 150 may be a transmissive electrode, a translucent electrode, or a reflective electrode. The second electrode 150 may have a single-layered structure or a multi-layered structure.

The emission layer 130 may include a host and a dopant.

In a non-limiting example, the emission layer 130 may include the dopant in an amount of about 0.01 parts by weight to about 15.00 parts by weight, or about 0.01 parts by weight to about 12.00 parts by weight, based on 100 parts by weight of the host.

The emission layer 130 may emit a red light, a green light, a blue light, and/or a white light. For example, the emission layer 130 may emit a blue light.

In some embodiments, an emission half width (full width at half maximum) of the blue light may be about 30 nm or less, or about 28 nm or less.

For example, the emission layer 130 may include a host material such as an anthracene derivative, a pyrene derivative, a fluoranthene derivative, a chrysene derivative, a dihydrobenzoanthracene derivative, a triphenylene derivative, or the like.

In some embodiments, the emission layer 130 may include, e.g., a host material represented by Chemical Formula FH. For example, the compound represented by Chemical Formula FH may be used as a fluorescent host material.

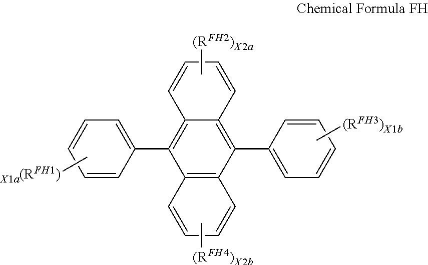

In Chemical Formula FH, R^{FH1} to R^{FH4} may each independently be a hydrogen atom, a deuterium atom, a halogen atom, a substituted or unsubstituted silyl group, a substituted or unsubstituted thio group, a substituted or unsubstituted oxy group, a substituted or unsubstituted C₁-C₁₀ alkyl group, a substituted or unsubstituted C₂-C₁₀ alkenyl group, a substituted or unsubstituted C₆-C₃₀ aryl group, a substituted or unsubstituted C₁-C₃₀ heteroaryl group, or a cyclic group formed through a combination thereof. In an embodiment, in Chemical Formula FH, at least one of R^{FH1} to R^{FH4} may form a condensed ring with a bonded benzene ring.

In Chemical Formula FH, x1a and x1b may each independently be an integer from 0 to 5; and x2a and x2b may each independently be an integer from 0 to 4. When x1a, x1b, x2a, and x2b are each 2 or more, two or more of each of R^{FH1} to R^{FH4} may be the same as or different from each other.

In some embodiments, the emission layer 130 may include, e.g., a host material represented by Chemical Formula PH. For example, the compound represented by Chemical Formula PH may be used as a host material for a phosphorescent device.

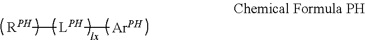

In Chemical Formula PH, R^{PH} may be a substituted or unsubstituted carbazole group. L^{PH} may be a direct linkage, a substituted or unsubstituted C₆-C₃₀ arylene group, or a substituted or unsubstituted C₁-C₃₀ heteroarylene group. Ar^{PH} may be a substituted or unsubstituted C₆-C₃₀ aryl group, or a substituted or unsubstituted C₁-C₃₀ heteroaryl group.

As described above in the definition of terminology, the term “C₆-C₃₀ aryl group” may encompass a group in which multiple aryl rings are condensed or bonded through a cyclic group (e.g., an alicyclic hydrocarbon ring). For example, a C₆-C₃₀ aryl group may include a fluorenyl group.

As described above in the definition of terminology, the term “C₁-C₃₀ heteroaryl group” may encompass a group in which multiple aryl rings are condensed or bonded through a heterocyclic ring. For example, a C₁-C₃₀ heteroaryl group may include a carbazole group, a dibenzofuran group, a dibenzothiophene group, or the like. In an embodiment, a C₁-C₃₀ heteroaryl group may be a group in which multiple aryl rings are condensed or bonded to each other through the same or different heterocyclic rings.

In an embodiment, a substituent included in Ar^{PH} may include a silyl group represented by —Si(R^{sa})(R^{sb})(R^{sc}); and R^{sa}, R^{sb}, and R^{sc} may each independently be hydrogen, deuterium, halogen, a hydroxyl group, a C₁-C₆₀ alkyl group, a C₁-C₆₀ alkoxy group, a C₁-C₆₀ alkylthio group, a C₆-C₆₀ aryl group, or a C₁-C₃₀ heteroaryl group. At least one of R^{sa}, R^{sb}, and R^{sc} may be a C₆-C₆₀ aryl group or a C₁-C₃₀ heteroaryl group. For example, R^{sa}, R^{sb} and R^{sc} may each independently be a C₆-C₆₀ aryl group or a C₁-C₃₀ heteroaryl group.

In Chemical Formula PH, 1× may be an integer from 0 to 10. When 1× is 2 or more, two or more of L^{PH} may be the same as or different from each other.

The emission layer 130 may include, e.g., BCPDS (bis(4-(9H-carbazol-9-yl) phenyl) diphenylsilane), POPCPA ((4-(1-(4-(diphenylamino) phenyl) cyclohexyl) phenyl) diphenyl-phosphine oxide), DPEPO (bis[2-(diphenylphosphino)phenyl]ether oxide), mCBP (3,3′-di(9H-carbazol-9-yl)-1,1′-biphenyl), CBP (4,4′-bis(N-carbazolyl)-1,1′-biphenyl), mCP (1,3-bis(carbazol-9-yl)benzene), PPF (2,8-bis(diphenylphosphoryl) dibenzo[b,d]furan), TCTA (4,4′,4″-tris(carbazol-9-yl)-triphenylamine), TPBi (1,3,5-tris(1-phenyl-1H-benzo[d]imidazole-2-yl)benzene), Alq₃ (tris(8-hydroxyquinolino) aluminum), ADN (9,10-di(naphthalene-2-yl)anthracene), TBADN (2-tert-butyl-9,10-di(naphth-2-yl)anthracene), DSA (distyrylarylene), CDBP (4,4′-bis(9-carbazolyl)-2,2′-dimethyl-biphenyl), MADN (2-methyl-9,10-bis(naphthalen-2-yl)anthracene), CP1 (hexaphenyl cyclotriphosphazene), UGH2 (1,4-bis(triphenylsilyl)benzene), DPSiO₃ (hexaphenylcyclotrisiloxane), DPSiO₄ (octaphenylcyclotetrasiloxane), or the like, as a host material.

In an embodiment, in the emission layer 130, the host may include one of the materials as described above, or any combination thereof.

Non-limiting examples of the host material are as follows:

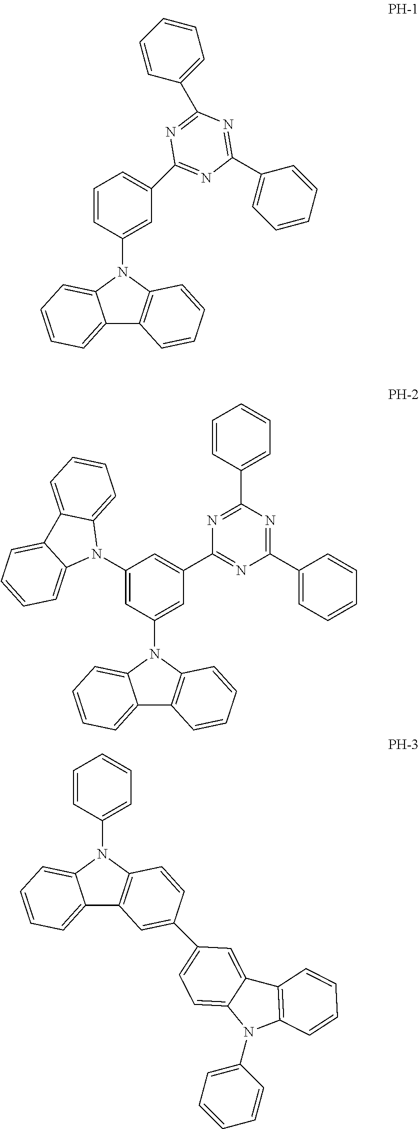

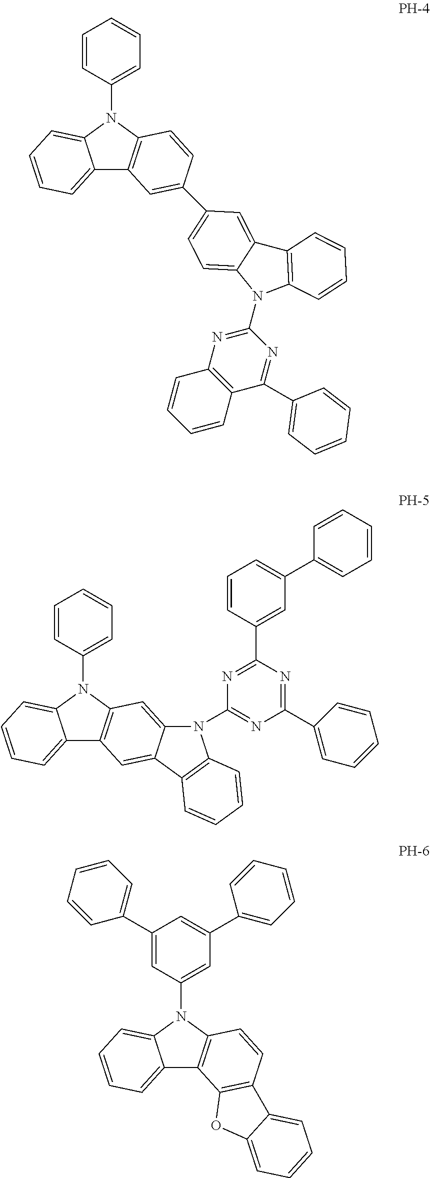 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-c3ccccc3)cc(-n3c4ccccc4c4c5oc6ccccc6c5ccc43)c2)cc1]

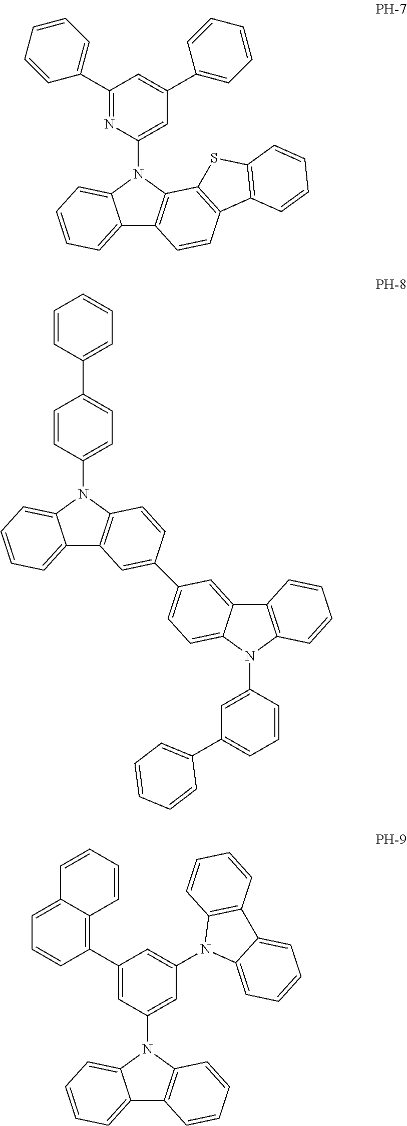 [SMILES: c1ccc(-c2cc(-c3ccccc3)nc(-n3c4ccccc4c4ccc5c6ccccc6sc5c43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

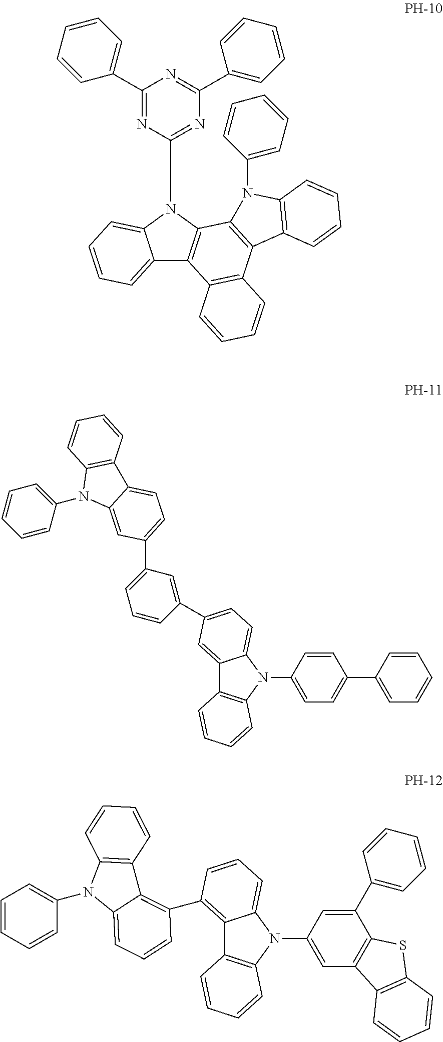 [SMILES: c1ccc(-c2nc(-c3ccccc3)nc(-n3c4ccccc4c4c5ccccc5c5c6ccccc6n(-c6ccccc6)c5c43)n2)cc1 ; c1ccc(-c2ccc(-n3c4ccccc4c4cc(-c5cccc(-c6ccc7c8ccccc8n(-c8ccccc8)c7c6)c5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

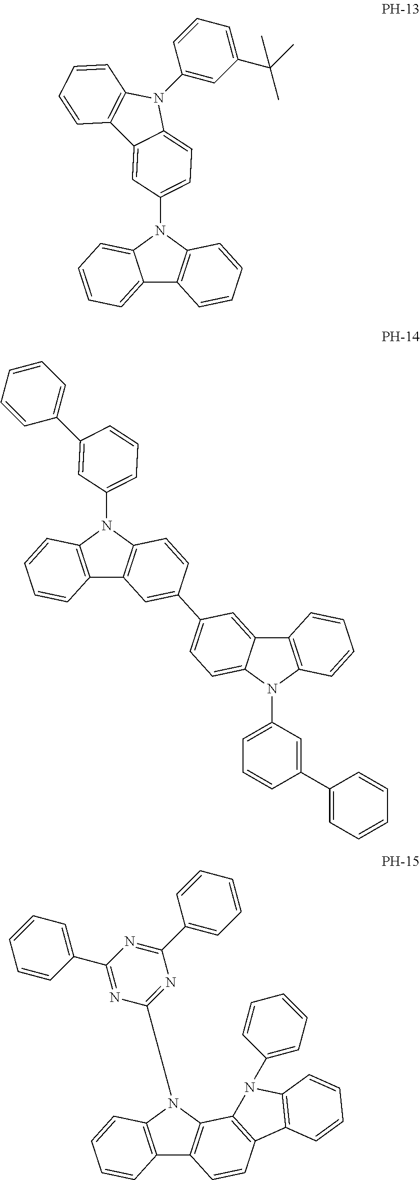 [SMILES: CC(C)(C)c1cccc(-n2c3ccccc3c3cc(-n4c5ccccc5c5ccccc54)ccc32)c1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2nc(-c3ccccc3)nc(-n3c4ccccc4c4ccc5c6ccccc6n(-c6ccccc6)c5c43)n2)cc1]

The emission layer 130 may further include a dopant interacting with the host material.

In some embodiments, the emission layer 130 may include a dopant represented by Chemical Formula FD. For example, the compound represented by Chemical Formula FD may be used as a fluorescent dopant.

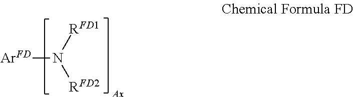

In Chemical Formula FD, Ar^{FD}, R^{FD1}, and R^{FD2} may each independently be a substituted or unsubstituted C₃-C₆₀ carbocyclic group, or a substituted or unsubstituted C₁-C₆₀ heterocyclic group. Ax may be an integer from 1 to 6.

In some embodiments, Ar^{FD} may include a condensed ring structure in which three or more aryl rings or benzene rings are condensed together (e.g., an anthracene group, a chrysene group, a pyrene group, etc.).

In some embodiments, the emission layer 130 may include a phosphorescent dopant. The phosphorescent dopant may include an organometallic compound that includes a central metal and at least one ligand bonded to the central metal via a coordination bond. The central metal may include, e.g., a transition metal, and the ligand may include, e.g., a monodentate ligand, a bidentate ligand, a tridentate ligand, a tetradentate ligand, a pentadentate ligand, a hexadentate ligand, or a combination thereof.

The phosphorescent dopant may include, e.g., a compound represented by Chemical Formula PD.

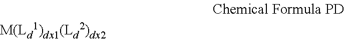

In Chemical Formula PD, M may be a transition metal atom, e.g., iridium (Ir), platinum (Pt), palladium (Pd), osmium (Os), titanium (Ti), gold (Au), hafnium (Hf), europium (Eu), terbium (Tb), rhodium (Rh), rhenium (Re), ruthenium (Ru), copper (Cu), or thulium (Tm).

In Chemical Formula PD, L_{d}¹ may be a ligand represented by Chemical Formula LD1.

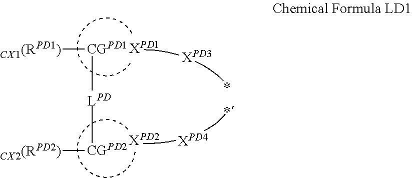

In Chemical Formula LD1, X^{PD1} and X^{PD2} may each independently be C or N.

In an embodiment, one of X^{PD1} and X^{PD2} may be C and the other of X^{PD1} and X^{PD2} may be N. In an embodiment, X^{PD1} and X^{PD2} may each be N.

In Chemical Formula LD1, CG^{PD1} and CG^{PD2} may each independently be a substituted or unsubstituted C₃-C₆₀ carbocyclic group, or a substituted or unsubstituted C₁-C⁶⁰ heterocyclic group. For example, CG^{PD1} and CG^{PD2} may each independently be a pyrrole group, a pyrazole group, an imidazole group, a triazole group, an oxazole group, an isoxazole group, a thiazole group, an isothiazole group, an oxadiazole group or a thiadiazole group, a benzene group, a pyridine group, a pyrimidine group, a naphthalene group, a dibenzofuran group, a dibenzothiophene group, a carbazole group, a fluorene group, a dibenzosilole group, a naphthobenzofuran group, a naphthobenzothiophene group, a benzocarbazole group, a benzofluorene group, a naphthobenzosilole group, a dinaphthofuran group, a dinaphthothiophene group, a dibenzocarbazole group, a dibenzofluorene group, a dinaphthosilole group, an azadibenzofuran group, an azadibenzothiophene group, an azacarbazole group, an azafluorene group, an azadibenzosilole group, an azanaphthobenzofuran group, an azanaphthobenzothiophene group, an azabenzocarbazole group, an azabenzofluorene group, an azanaphthobenzosilole group, an azadinaphthofuran group, an azadinaphthothiophene group, an azadibenzocarbazole group, an azadibenzofluorene group, or an azadinaphthosilole group.

In Chemical Formula LD1, L^{PD} may be a single bond, a substituted or unsubstituted methylene group, a substituted or unsubstituted ethylene group, *—O—*′, *—S—*′, *—C(═O)—*′, *—N(R^{PD3})—*′, *—C(R^{PD4})—*, or *═C(R^{PD5})—*′.

In Chemical Formula LD1, X^{PD3} and X^{PD4} may each independently be a chemical bond, O, S, N(R^{PD6}), B(R^{PD7}), P(R^{PD8}), C(R^{PD9})(R^{PD10}), or Si(R^{PD11})(R^{PD12}). The chemical bond may be, e.g., a covalent bond or a coordination bond.

In Chemical Formula LD1, R^{PD1} and R^{PD2} may each independently be hydrogen, deuterium, —F, —Cl, —Br, —I, —SF₅, —OH, —CN, —NO₂, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₁-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₁-C₆₀ heteroaryloxy group, a substituted or unsubstituted C₁-C₆₀ heteroarylthio group, a substituted or unsubstituted C₈-C₆₀ condensed polycyclic group, a substituted or unsubstituted silyl group, a substituted or unsubstituted amine group, a substituted or unsubstituted aniline group, —B(R^{PD13})(R^{PD14}), —C(═O)(R^{PD15}), —S(═O)₂(R^{PD16}), —P(R^{PD17})(R^{PD18}), or —P(═O)(R^{PD17})(R^{PD18}). The silyl group may be represented by —Si(R^{sa})(R^{sb})(R^{sc}), as explained above.

R^{PD3} to R^{PD18} may each independently be hydrogen, deuterium, —F, —Cl, —Br, —I, —SF₅, —OH, —CN, —NO₂, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₁-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₁-C₆₀ heteroaryloxy group, a substituted or unsubstituted C₁-C₆₀ heteroarylthio group, or a substituted or unsubstituted C₈-C₆₀ condensed polycyclic group.

In Chemical Formula LD1, cx1 and cx2 may each independently be an integer from 0 to 10. When at least one of cx1 and cx2 is 2 or more, two or more of R^{PD1} or two or more of R^{PD2} may be the same as or different from each other.

The symbols -* and -*′ each represent a binding site where the ligand represented by Chemical Formula LD1 bonds to M.

In Chemical Formula PD, dx1 may be an integer from 1 to 3. When dx1 is 2 or 3, two or three of L_{d}¹ may be the same as or different from each other. Among two or three of L_{d}¹, CG^{PD1} and/or CG^{PD2} adjacent to each other may be connected to each other through a connecting group such as L^{PD1} L^{PD2}, or the like. The connecting group such as L^{PD1}, L^{PD2} etc., may each independently be the same as defined in connection with L^{PD}.

In Chemical Formula PD, L_{d}² may be an organic ligand. L_{d}² may include, e.g., a halogen group, CO, NO, CS, a picolinate group, an acetate group, an oxalate group, a diketone group, an isonitrile group, a isothiocyanato-N group, a thiosulphato-S group, an alkyl phosphine group, a phenylphosphine group, an aryl phosphine group, a phosphine oxide group, a phosphite group, or a combination thereof.

In Chemical Formula PD, dx2 is an integer of 0 to 4. When dx2 is 2 or more, two or more of L_{d}² may be the same as or different from each other.

Non-limiting examples of the phosphorescent dopant are as follows:

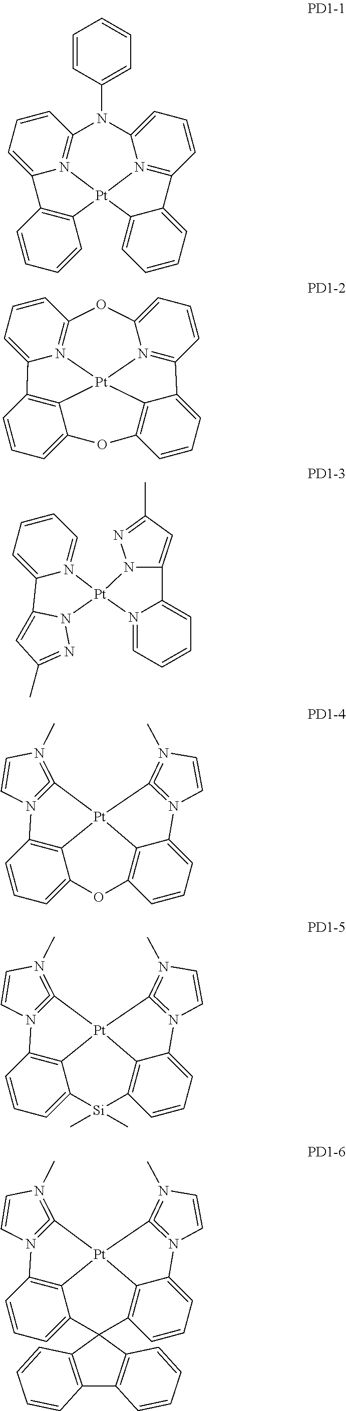

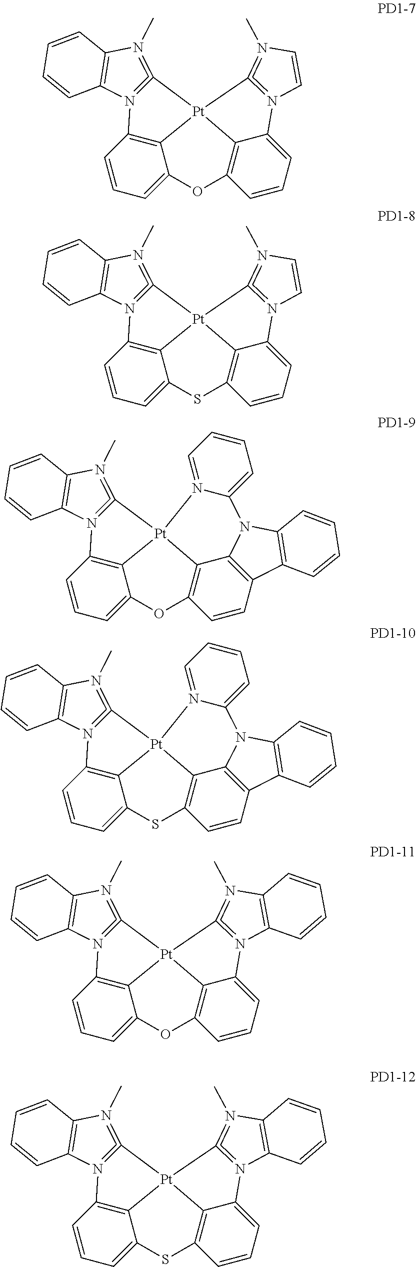

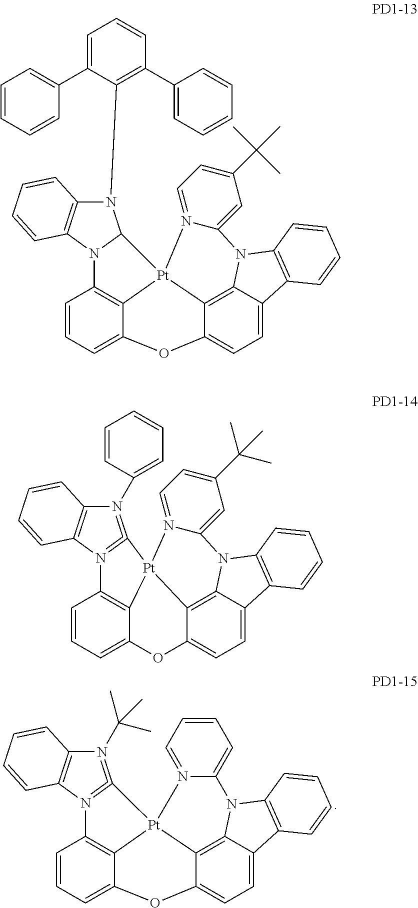

In some embodiments, the emission layer 130 may include a styryl derivative (e.g., 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (NBDAVBi), etc.), 4,4′-bis[2-(4-(N,N-diphenylamino)phenyl)vinyl]biphenyl (DPAVBi), perylene or a derivative thereof (e.g., 2,5,8,11-tetra-t-butylperylene (TBP)), pyrene or a derivative thereof (e.g., 1,1-dipyrene, 1,4-dipyrenylbenzene, 1,4-bis(N,N-diphenylamino)pyrene), or the like), or the like, as a fluorescent dopant material.

The emission layer 130 may include a metal complex that includes iridium (Ir), platinum (Pt), osmium (Os), gold (Au), titanium (Ti), zirconium (Zr), hafnium (Hf), europium (Eu), terbium (Tb), or thulium (Tm) as a phosphorescent dopant, in addition to the materials described above. For example, FIrpic (iridium(III) bis(4,6-difluorophenylpyridinato-N,C2′)picolinate), FIr6 (bis(2,4-difluorophenylpyridinato)-tetrakis(1-pyrazolyl)borate iridium(III)), PtOEP (platinum octaethylporphyrin), or the like, may be used as the phosphorescent dopant.

In embodiments, the emission layer 130 may include a boron-containing dopant represented by Chemical Formula BD.

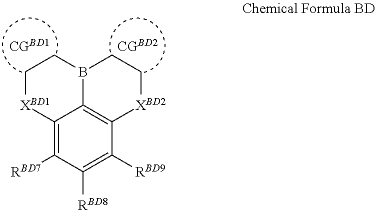

In Chemical Formula BD, X^{BD1} and X^{BD2} may each independently be N(R^{BD1}), P(R^{BD2}) C(R^{BD3})(R^{BD4}), Si(R^{BD5})(R^{BD6}), S, or O. In an embodiment, X^{BD1} and X^{BD2} may each be N. R^{BD1} to R^{BD6} may each independently be hydrogen, deuterium, a substituted or unsubstituted C₁-C₂₀ alkyl group, a substituted or unsubstituted C₆-C₃₀ aryl group, or a substituted or unsubstituted C₁-C₃₀ heteroaryl group. R^{BD7}, R^{BD8}, and R^{BD9} may each independently be hydrogen, deuterium, halogen, a cyano group, a substituted or unsubstituted amine group, a substituted or unsubstituted boryl group (boron group), a substituted or unsubstituted oxy group, a substituted or unsubstituted thio group, a substituted or unsubstituted C₁-C₂₀ alkyl group, a substituted or unsubstituted C₆-C₃₀ aryl group, or a substituted or unsubstituted C₁-C₃₀ heteroaryl group. R^{BD7}, R^{BD8}, and/or R^{BD9} may be bonded to an adjacent group to form a ring.

In Chemical Formula BD, CG^{BD1} and CG^{BD2} each represents a cyclic group, and CG^{BD1} and CG^{BD2} may each independently be a substituted or unsubstituted C₃-C₆₀ carbocyclic group, or a substituted or unsubstituted C₁-C₆₀ heterocyclic group. In some embodiments, CG^{BD1} and CG^{BD2} may each independently be a substituted or unsubstituted C₆-C₃₀ aryl group, or a substituted or unsubstituted C₁-C₃₀ heteroaryl group.

In an embodiment, CG^{BD1} and CG^{BD2} may each independently be a substituted or unsubstituted benzene ring. In this case, the boron-containing dopant may serve as a thermally activated delayed fluorescence (TADF) dopant.

In an embodiment, one of CG^{BD1} or CG^{BD2} may be a non-condensed aryl group or a non-condensed heteroaryl group, and the other one thereof may be a condensed polycyclic aryl group or a condensed polycyclic heteroaryl group. In this case, the boron-containing dopant may serve as a fluorescent dopant.

In an embodiment, the emission layer 130 may include one of the dopant materials as described above, or any combination thereof.

In some embodiments, the emission layer 130 may include two or more host materials. For example, the emission layer 130 may include a hole transporting host and an electron transporting host. In this case, the emission layer 130 may include a hole transporting host, an electron transporting host, a photosensitive agent, and a dopant. In example embodiments, the hole transporting host and the electron transporting host may form an exciplex, and energy may be transferred from the exciplex to the photosensitive agent and from the photosensitive agent to the dopant, thereby inducing a light emission.

Non-limiting examples of the hole transporting host may include a compound represented by Chemical Formula HT as described herein. Non-limiting examples of the electron transporting host may include a compound represented by Chemical Formula ET as described herein.

In some embodiments, the emission layer 130 may include quantum dots. A quantum dot may include a Group II-VI compound, a Group III-VI compound, a Group I-III-VI compound, a Group III-V group compound, a Group III-II-V group compound, a Group IV-VI compound, a Group IV element, a Group IV compound, or a combination thereof.

The quantum dot may include a core that includes the compound as described above, and a shell surrounding the core. The shell may include an inorganic oxide or a semiconductor compound. Examples of the semiconductor compound may include CdS, CdSe, CdTe, ZnS, ZnSe, ZnTe, ZnSe, ZnTeS, GaAs, GaP, GaSb, HgS, HgSe, HgTe, InAs, InP, InGaP, InSb, AlAs, AlP, AlSb, or the like.

In example embodiments, a color of light from a quantum dot may be adjusted according to a particle size of the quantum dot. The quantum dot may be a blue quantum dot, a red quantum dot, or a green quantum dot.

The hole transfer region 120 may be formed between the first electrode 110 and the emission layer 130. The hole transfer region 120 may have a single-layered structure or a multi-layered structure including different materials.

The hole transfer region 120 may include the above-described condensed ring compound. For example, the condensed ring compound may be used as a p-dopant.

The hole transfer region 120 may include a hole injection layer, a hole transport layer, and/or an electron blocking layer, and may further include an auxiliary emission layer.

In some embodiments, as illustrated in FIG. 2, the hole transfer region 120 may include a hole injection layer 122 and a hole transport layer 124, sequentially stacked from the first electrode 110.

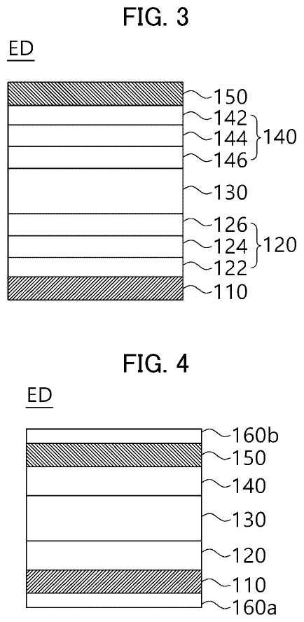

In some embodiments, as illustrated in FIG. 3, the hole transfer region 120 may include a hole injection layer 122, a hole transport layer 124, and an electron blocking layer 126, sequentially stacked from the first electrode 110. The electron blocking layer 126 may block an electron transfer from the electron transfer region 140 to the hole transfer region 120. Accordingly, the generation of excitons in the emission layer 130 may be increased, and light-emission efficiency may be further increased.

In some embodiments, the hole injection layer 122 may include the condensed ring compound represented by Chemical Formula 1 in an amount of 1 wt % to 5 wt % based on the total weight thereof.

In some embodiments, the hole transfer region 120 may further include a compound described herein.

For example, the hole transfer region 120 may further include a compound represented by Chemical Formula HT.

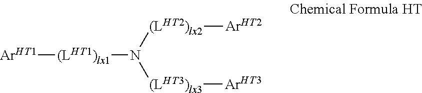

In Chemical Formula HT, L^{HT1} L^{HT2}, and L^{HT3} may each independently be a direct linkage, a substituted or unsubstituted C₆-C₃₀ arylene group, or a substituted or unsubstituted C₁-C₃₀ heteroarylene group.

In Chemical Formula HT, lx1 to lx3 may each independently be an integer from 0 to 10. When lx1, lx2, or lx3 is 2 or more, two or more of each of L^{HT1} L^{HT2}, or L^{HT3} may be directly connected by, e.g., carbon atoms (e.g., sp² carbon atoms) of each aryl ring, to form a substituted or unsubstituted C₆-C₃₀ arylene group, or a substituted or unsubstituted C₁-C₃₀ heteroarylene group.

In Chemical Formula HT, Ar^{HT1} and Ar^{HT2} may each independently be a substituted or unsubstituted C₆-C₃₀ aryl group, or a substituted or unsubstituted C₁-C₃₀ heteroaryl group. Ar^{HT3} may be a substituted or unsubstituted C₆-C₃₀ aryl group.

In an embodiment, the compound represented by Chemical Formula HT may be a monoamine compound. In an embodiment, the compound represented by Chemical Formula HT may be a diamine compound in which at least one of Ar^{HT1} to Ar^{HT3} includes an amine group as a substituent.

In some embodiments, the compound represented by Chemical Formula HT may be a carbazole-containing compound in which at least one of Ar^{HT1} and Ar^{HT2} includes a substituted or unsubstituted carbazole group, or a fluorene-containing compound in which at least one of Ar^{HT1} and Ar^{HT2} includes a substituted or unsubstituted fluorene group.

In some embodiments, two adjacent groups among Ar^{HT1} to Ar^{HT3} may be condensed together to form a ring.

In non-limiting examples, the hole transfer region 120 may further include at least one of compounds as follows, but embodiments are not limited thereto:

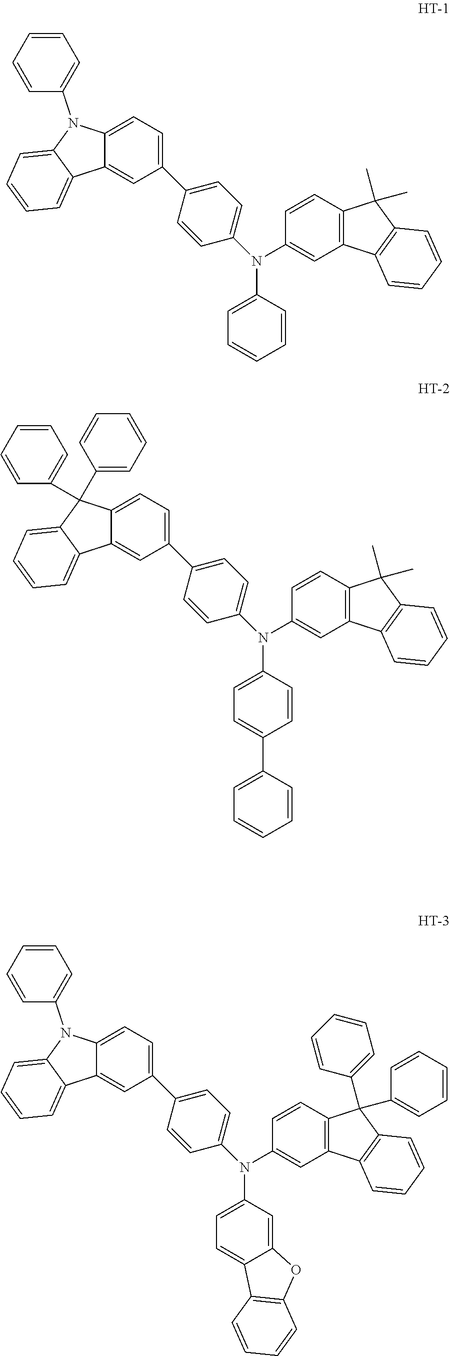 [SMILES: CC1(C)c2ccccc2-c2cc(N(c3ccccc3)c3ccc(-c4ccc5c(c4)c4ccccc4n5-c4ccccc4)cc3)ccc21 ; CC1(C)c2ccccc2-c2cc(N(c3ccc(-c4ccccc4)cc3)c3ccc(-c4ccc5c(c4)-c4ccccc4C5(c4ccccc4)c4ccccc4)cc3)ccc21 ; c1ccc(-n2c3ccccc3c3cc(-c4ccc(N(c5ccc6c(c5)-c5ccccc5C6(c5ccccc5)c5ccccc5)c5ccc6c(c5)oc5ccccc56)cc4)ccc32)cc1]

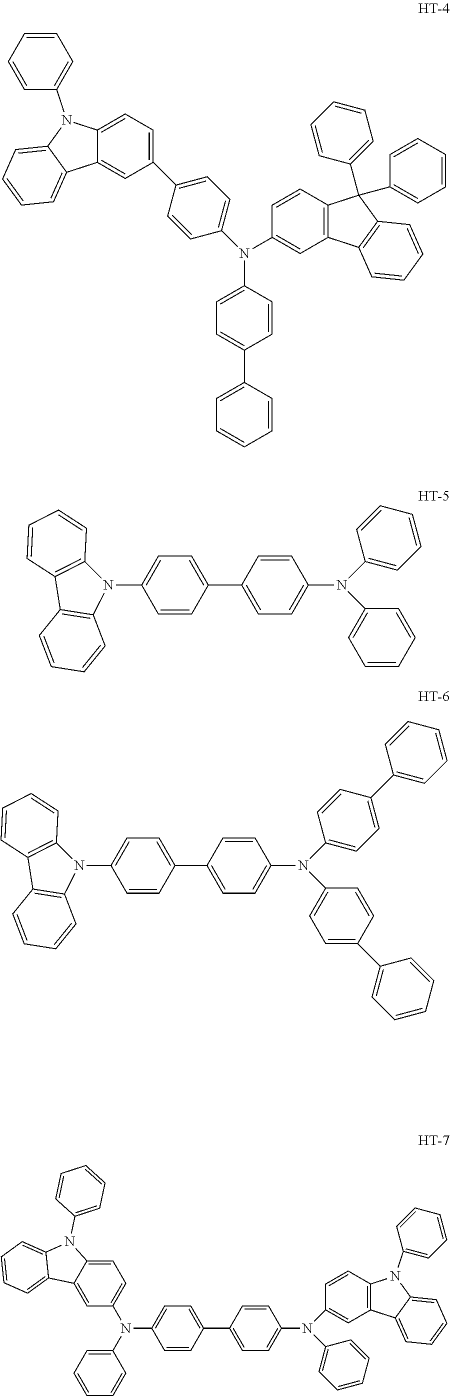 [SMILES: c1ccc(-c2ccc(N(c3ccc(-c4ccc5c(c4)c4ccccc4n5-c4ccccc4)cc3)c3ccc4c(c3)-c3ccccc3C4(c3ccccc3)c3ccccc3)cc2)cc1 ; c1ccc(N(c2ccccc2)c2ccc(-c3ccc(-n4c5ccccc5c5ccccc54)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

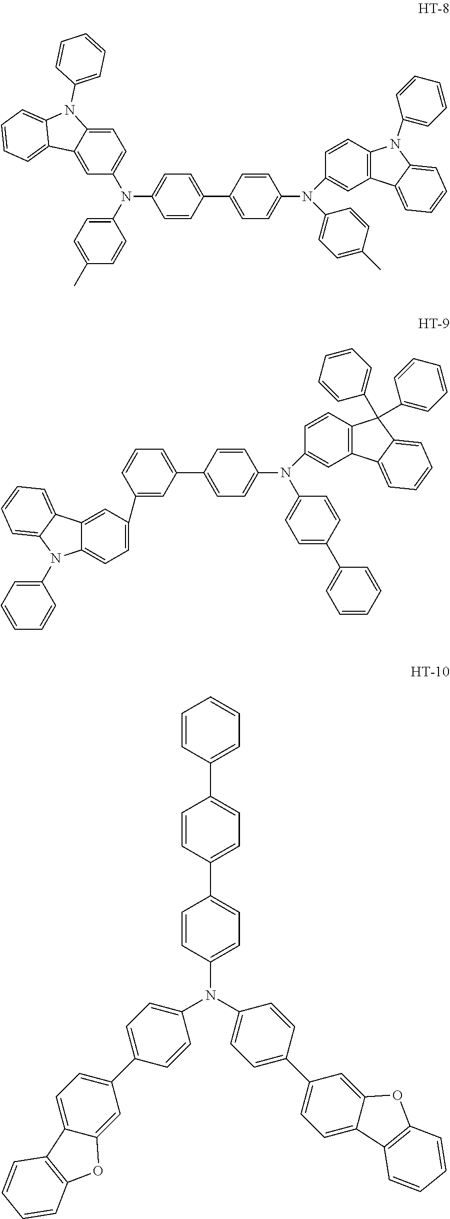 [SMILES: Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; c1ccc(-c2ccc(N(c3ccc(-c4cccc(-c5ccc6c(c5)c5ccccc5n6-c5ccccc5)c4)cc3)c3ccc4c(c3)-c3ccccc3C4(c3ccccc3)c3ccccc3)cc2)cc1 ; c1ccc(-c2ccc(-c3ccc(N(c4ccc(-c5ccc6c(c5)oc5ccccc56)cc4)c4ccc(-c5ccc6c(c5)oc5ccccc56)cc4)cc3)cc2)cc1]

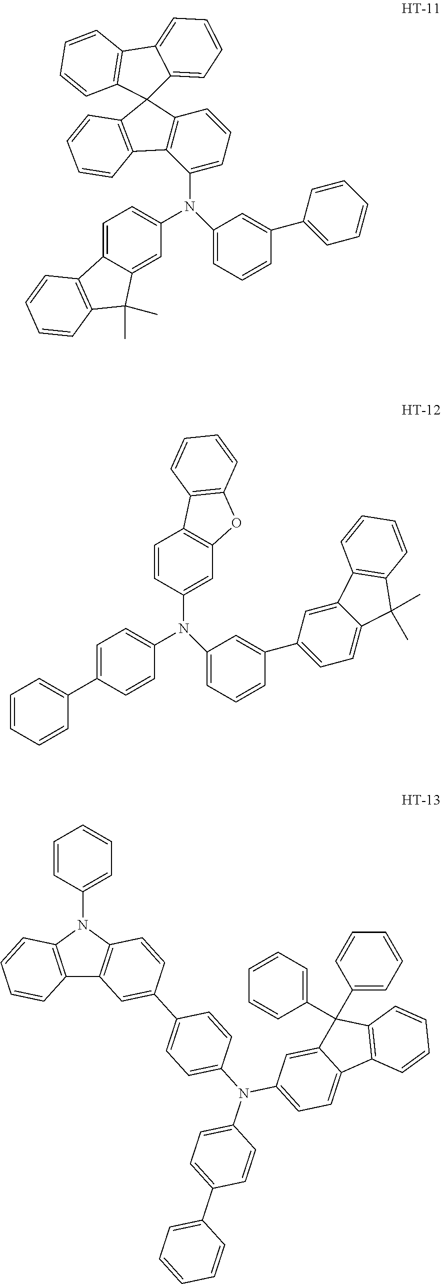 [SMILES: CC1(C)c2ccccc2-c2ccc(N(c3cccc(-c4ccccc4)c3)c3cccc4c3-c3ccccc3C43c4ccccc4-c4ccccc43)cc21 ; CC1(C)c2ccccc2-c2cc(-c3cccc(N(c4ccc(-c5ccccc5)cc4)c4ccc5c(c4)oc4ccccc45)c3)ccc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

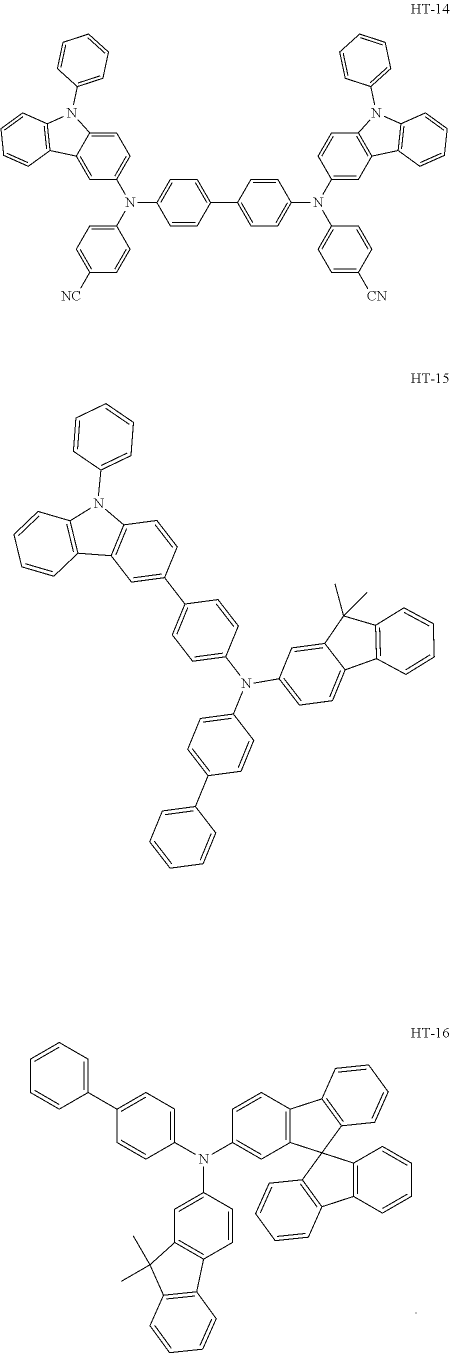 [SMILES: N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccc(-c4ccccc4)cc3)c3ccc4c(c3)C3(c5ccccc5-c5ccccc53)c3ccccc3-4)cc21]

For example, the hole transfer region 120 may include m-MTDATA (4,4′,4″-[tris(3-methylphenyl)phenylamino]triphenylamine), TDATA (4,4′4″-tris(N,N-diphenylamino)triphenylamine), 2-TNATA (4,4′,4″-tris[N(2-naphthyl)-N-phenylamino]-triphenylamine), NPB (N,N′-di(naphthalene-1-yl)-N,N′-diphenyl-benzidine), TPD (N,N′-bis(3-methylphenyl)-N,N′-diphenyl-[1,1′-biphenyl]-4,4′-diamine), Spiro-TPD, Spiro-NPB, DNTPD (N¹,N^{1′}-([1,1′-biphenyl]-4,4′-diyl)bis(N¹-phenyl-N⁴,N⁴-di-m-tolylbenzene-1,4-diamine), TAPC (4,4′-cyclohexylidene bis[N,N-bis(4-methylphenyl)benzenamine]), HMTPD (4,4′-bis[N,N′-(3-tolyl)amino]-3,3′-dimethylbiphenyl), TCTA (4,4′,4″-tris(N-carbazolyl)triphenylamine), PANI/DBSA (polyaniline/dodecylbenzenesulfonic acid), PEDOT/PSS (poly(3,4-ethylenedioxythiophene)/poly(4-styrenesulfonate)), PANI/CSA (polyaniline/Camphor sulfonic acid), PANI/PSS (polyaniline/poly(4-styrenesulfonate)), a phthalocyanine compound, a carbazole compound (N-phenylcarbazole, polyvinylcarbazole, CzSi (9-(4-tert-butylphenyl)-3,6-bis(triphenylsilyl)-9H-carbazole), CCP (9-phenyl-9H-3,9′-bicarbazole), mDCP (1,3-bis(1,8-dimethyl-9H-carbazol-9-yl)benzene), or the like), a fluorene compound, or the like. The hole transfer region 120 may include one of the hole transfer materials described herein, or a combination thereof.

The hole transfer materials described herein may be included in at least one of the hole injection layer 122, the hole transport layer 124, or the electron blocking layer 126.

The hole transfer region 120 may further include a charge generating material. The charge generating material may be a dopant material such as a p-dopant, so that conductivity of the hole transfer region 120 may be improved.

Examples of the dopant material may include a halogenated metal compound such as LiF, NaCl, CsF, RbCl, RbI, CuI, or KI; a quinone derivative such as TCNQ (tetracyanoquinodimethane), F4-TCNQ (2,3,5,6-tetrafluoro-7,7,8,8-tetracyanoquinodimethane), or the like; a cyano-containing compound such as HATCN (dipyrazino[2,3-f: 2′,3′-h]quinoxaline-2,3,6,7,10,11-hexacarbonitrile), NDP9 (4-[[2,3-bis[cyano-(4-cyano-2,3,5,6-tetrafluorophenyl)methylidene]cyclopropylidene]-cyanomethyl]-2,3,5,6-tetrafluorobenzonitrile), or the like; a tungsten (W) oxide; a molybdenum (Mo) oxide; or the like. The hole transfer region 120 may include one of the dopant materials described herein, or a combination thereof.

A thickness of the hole transfer region 120 may be about 100 Å to about 10,000 Å. For example, the thickness of the hole transfer region 120 may be about 100 Å to about 1,500 Å.

When the hole transfer region 120 includes the hole injection layer 122 or the hole transport layer 124, a thickness of the hole injection layer 122 may be from about 100 Å to about 9,000 Å, from about 100 Å to about 3,000 Å, or from about 100 Å to about 1,000 Å. A thickness of the hole transport layer 124 may be from 50 Å to about 2,000 Å, from about 100 A to about 1,500 Å, from about 100 Å to about 1,000 Å, or from about 100 Å to about 600 Å.

In the thickness ranges described herein, hole transfer properties may be enhanced even at a low voltage operation, and a life-span of the device may be further improved.

Each layer of the hole transfer region 120 may be formed by a process such as a thermal evaporation deposition, a vacuum deposition, a spin coating, an inkjet printing, a laser printing, a casting, a laser thermal transfer, or the like.

The electron transfer region 140 may be disposed between the second electrode 150 and the emission layer 130. The electron transfer region 140 may have a single-layered, or a multi-layered structure including different materials.

The electron transfer region 140 may include an electron injection layer, an electron transport layer, and/or a hole blocking layer, and may further include an auxiliary emission layer.

In embodiments, as illustrated in FIG. 2, the electron transfer region 140 may include an electron injection layer 142 and an electron transport layer 144, stacked from the second electrode 150 to the emission layer 130.

In some embodiments, as illustrated in FIG. 3, the electron transfer region 140 may include an electron injection layer 142, an electron transport layer 144, and a hole blocking layer 146, sequentially stacked from the second electrode 150. The hole blocking layer 146 may block or suppress a hole transfer from the hole transfer region 120. Accordingly, emission energy and luminescence efficiency in the emission layer 130 may be further improved.

For example, the electron transfer region 140 may include a compound represented by Chemical Formula ET.

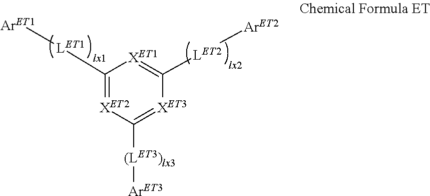

In Chemical Formula ET, at least one of X^{ET1} to X^{ET3} may be N; and the remainder of X^{ET1} to X^{ET3} may each independently be C(R^{ET}). R^{ET} may be a hydrogen atom, a deuterium atom, a substituted or unsubstituted C₁-C₂₀ alkyl group, a substituted or unsubstituted C₆-C₆₀ aryl group, or a substituted or unsubstituted C₁-C₆₀ heteroaryl group.

When one of X^{ET1} to X^{ET3} is N, the compound represented by Chemical Formula ET may include a pyridine group. When two of X^{ET1} to X^{ET3} are N, the compound represented by Chemical Formula ET may include a pyrimidine group. When X^{ET1} to X^{ET3} are each N, the compound represented by Chemical Formula ET may include a triazine group.

In Chemical Formula ET, lx1 to lx3 may each independently be an integer from 0 to 10. L^{ET1} to L^{ET3} may each independently be a direct linkage, a substituted or unsubstituted C₆-C₃₀ arylene group, or a substituted or unsubstituted C₁-C₃₀ heteroarylene group.

When lx1, lx2, or lx3 is 2 or more, two or more of each of L^{ET1}, L^{ET2}, or L^{ET3} respectively, may be directly linked together, e.g., by carbon atoms of each aryl ring (e.g., sp² carbon atoms), to form a substituted or unsubstituted C₆-C₃₀ arylene group, or a substituted or unsubstituted C₁-C₃₀ heteroarylene group.

In Chemical Formula ET, Ar^{ET1} to Ar^{ET3} may each independently be a hydrogen atom, a deuterium atom, a substituted or unsubstituted C₁-C₂₀ alkyl group, a substituted or unsubstituted C₆-C₃₀ aryl group, or a substituted or unsubstituted C₁-C₃₀ heteroaryl group. For example, Ar^{ET1} to Ar^{ET3} may each independently be a substituted or unsubstituted phenyl group, a substituted or unsubstituted carbazole group, a substituted or unsubstituted fluorene group, or a substituted or unsubstituted silyl group. The silyl group may be represented by —Si(R^{sa})(R^{sb})(R^{sc}), as explained herein.

Non-limiting examples of the electron transfer material included in the electron transfer region 140 include the following:

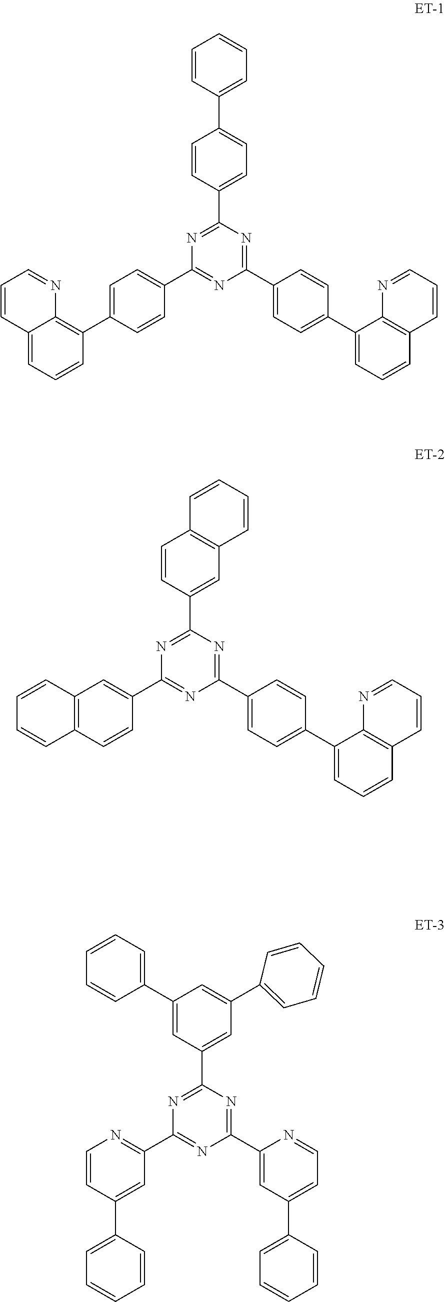 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; c1ccc(-c2cc(-c3ccccc3)cc(-c3nc(-c4cc(-c5ccccc5)ccn4)nc(-c4cc(-c5ccccc5)ccn4)n3)c2)cc1]

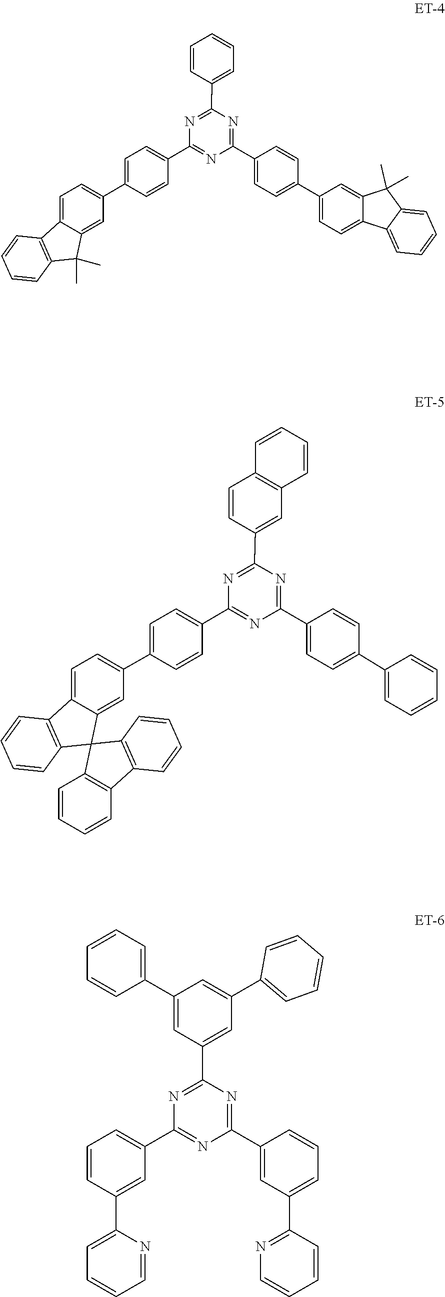 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3nc(-c4ccc(-c5ccc6c(c5)C5(c7ccccc7-c7ccccc75)c5ccccc5-6)cc4)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

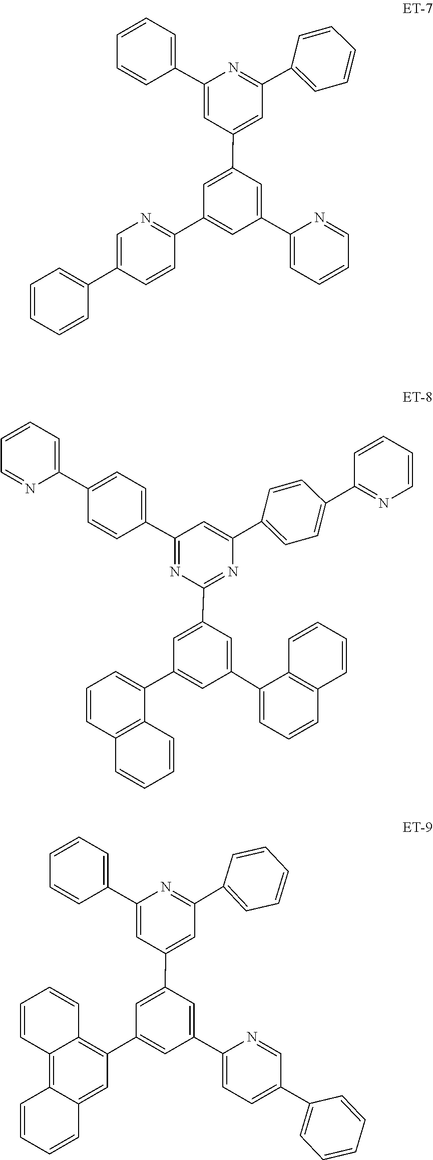 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

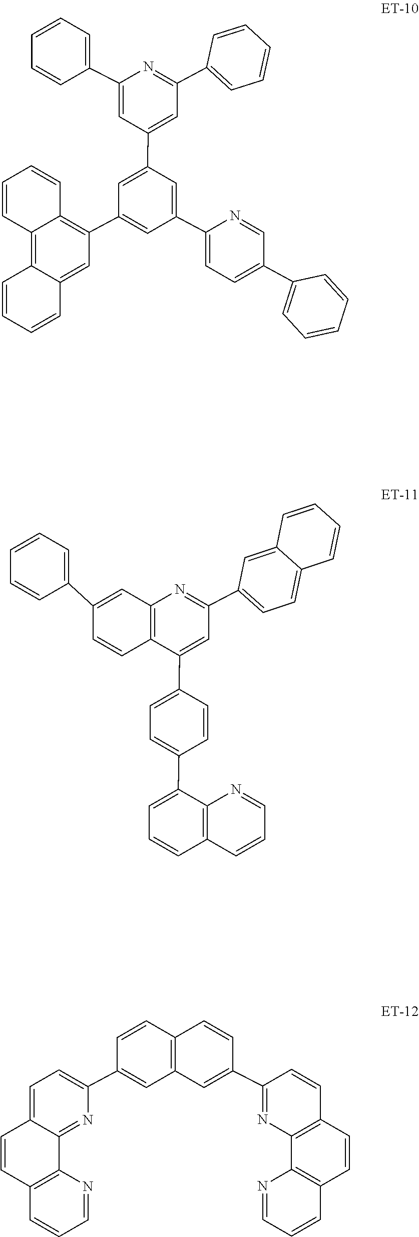 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

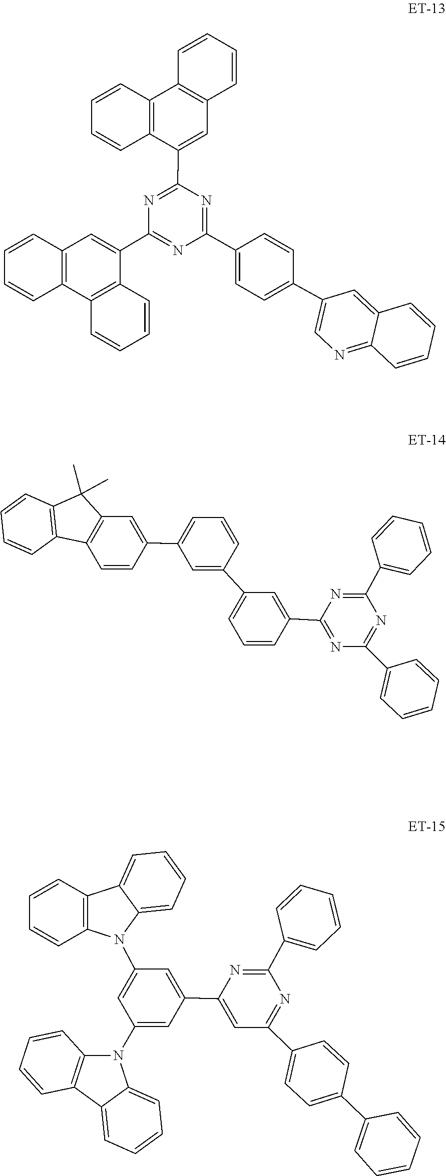 [SMILES: c1ccc2ncc(-c3ccc(-c4nc(-c5cc6ccccc6c6ccccc56)nc(-c5cc6ccccc6c6ccccc56)n4)cc3)cc2c1 ; CC1(C)c2ccccc2-c2ccc(-c3cccc(-c4cccc(-c5nc(-c6ccccc6)nc(-c6ccccc6)n5)c4)c3)cc21 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

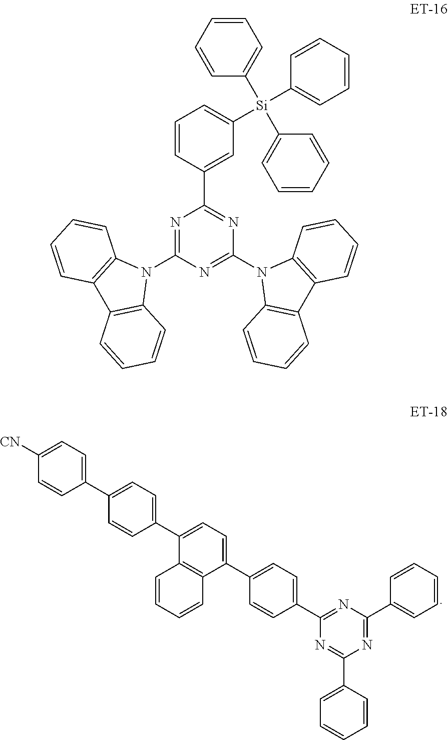

For example, the electron transfer region 140 may include an anthracene compound, Alq₃ (tris(8-hydroxyquinolinato)aluminum), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzimidazol-1-yl)phenyl)-9,10-dinaphthylanthracene, TPBi (1,3,5-Tri(1-phenyl-1H-benzo[d]imidazol-2-yl)benzene), BCP (2,9-dimethyl-4,7-diphenyl-1,10-phenanthroline), Bphen (4,7-diphenyl-1,10-phenanthroline), TAZ (3-(4-biphenylyl)-4-phenyl-5-tert-butylphenyl-1,2,4-triazole), NTAZ (4-(naphthalen-1-yl)-3,5-diphenyl-4H-1,2,4-triazole), tBu-PBD (2-(4-biphenylyl)-5-(4-tert-butylphenyl)-1,3,4-oxadiazole), BAlq (Bis(2-methyl-8-quinolinolato-N1,O8)-(1,1′-biphenyl-4-olato)aluminum), Bebg₂ (beryllium bis(benzoquinolin-10-olate)), AND (9,10-di(naphthalene-2-yl)anthracene), BmPyPhB (1,3-bis[3,5-di(pyridin-3-yl)phenyl]benzene), TSPO1 (diphenyl(4-(triphenylsilyl)phenyl)phosphine oxide), or the like. The electron transfer region 140 may include one of the electron transfer materials described herein, or a combination thereof.

The above-mentioned material may be included in at least one of the electron injection layer 142, the electron transport layer 144, or the hole blocking layer 146.

The electron transfer region 140 may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or a combination thereof. In an embodiment, the above-mentioned material may be included electron injection layer 142.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or the like, or any combination thereof.

The alkali metal-containing compound, the alkaline earth metal-containing compound, and the rare earth metal-containing compound may include an oxide, a halide (e.g., a fluoride, a chloride, a bromide, an iodide, or the like), a telluride, or a combination thereof of the alkali metal, the alkaline earth metal, and the rare earth metal, respectively.

The alkali metal complex, the alkaline earth metal complex, and the rare earth metal complex may include a metal ion such as an alkali metal ion, an alkaline earth metal ion or a rare earth metal ion, and a ligand bonded to the metal ion. The ligand may include, e.g., hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, cyclopentadiene, or a combination thereof.

A thickness of the electron transfer region 140 may be from about 100 Å to about 1,000 Å, e.g., from about 150 Å to about 500 Å.

When the electron transfer region 140 includes an electron injection layer 142 or an electron transport layer 144, a thickness of the electron injection layer 142 may be from about 1 Å to about 100 Å, from 1 Å to about 90 Å or from about 5 Å to about 50 Å, and a thickness of the electron transport layer 144 may be from 10 Å to about 900 Å, from about 10 Å to about 500 Å or from about 100 Å to about 400 Å.

Within any of the thickness ranges described herein, electron injection and electron transport properties may be further improved without an excessive increase in driving voltage, and stability of the electron transfer region 140 may be improved.

Each layer of the electron transfer region 140 may be formed by a process such as a thermal evaporation deposition, a vacuum deposition, a spin coating, an inkjet printing, a laser printing, a casting, a laser thermal transfer, or the like.

The light-emitting device ED may further include a capping layer. Light emission efficiency to outside of the light-emitting device ED may be improved through the capping layer.

As illustrated in FIG. 4, a second capping layer 160b may be formed on an outer surface of the second electrode 150. In some embodiments, a first capping layer 160a may be formed on an outer surface of the first electrode 110.

A refractive index of the first capping layer 160a and/or the second capping layer 160b may be about 1.6 or more. For example, the refractive index of the first capping layer 160a and/or the second capping layer 160b may be 1.6 or more, 1.8 or more, or 2.0 or more for a light in a wavelength range of 550 nm to 660 nm.

The first capping layer 160a and the second capping layer 160b may each be formed as an organic capping layer including an organic material, an inorganic capping layer including an inorganic material, or an organic-inorganic hybrid capping layer including both the organic and inorganic materials.

The first capping layer 160a and/or the second capping layer 160b may each have a single-layered structure or a multi-layered structure including different materials.

In some embodiments, the first capping layer 160a and the second capping layer 160b may each independently include a carbocyclic compound, a heterocyclic compound, an amine group-containing compound, a porphine derivative, a phthalocyanine derivative, a naphthalocyanine derivative, an alkaline metal complex, an alkaline earth metal complex, or the like. The first capping layer 160a and the second capping layer 160b may each independently include one of the aforementioned materials, or a combination thereof.

In an embodiment, the first capping layer 160a and/or the second capping layer 160b may each independently include an amine group-containing compound.

In a non-limiting example, the first capping layer 160a and/or the second capping layer 160b may include at least one of the compounds represented by Chemical Formulae P1 to P4, and/or at least one of the compounds HT-7, HT-8, or HT-14.

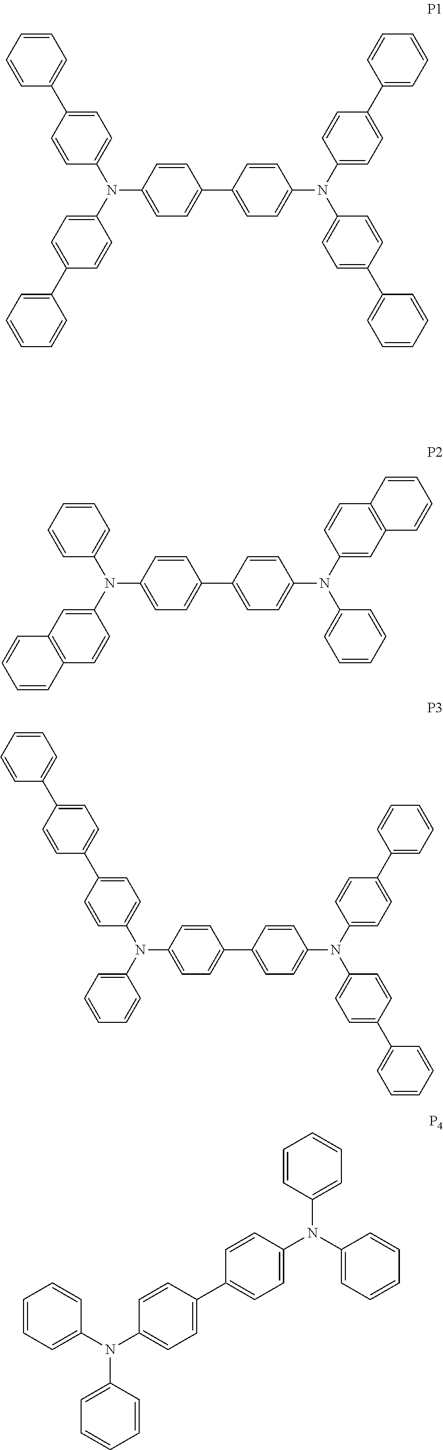 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccccc2)c2ccc(-c3ccc(N(c4ccccc4)c4ccccc4)cc3)cc2)cc1]

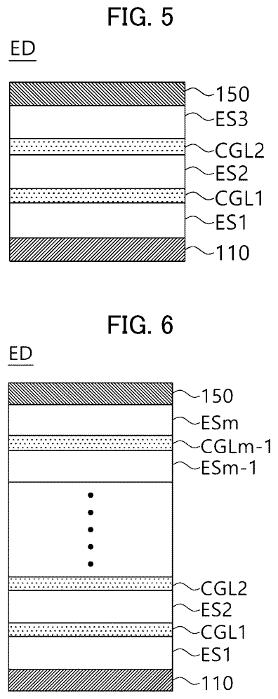

Referring to FIG. 5, the light-emitting device ED may include a plurality of light-emitting structures (e.g., the light-emitting structures ES1, ES2, and ES3). The light-emitting structures ES1, ES2, and ES3 may each include a stacked structure of the hole transfer region 120, the emission layer 130, and the electron transfer region 140, as described with reference to FIGS. 1 to 4. In example embodiments, the light-emitting device ED of FIG. 5 may be a light-emitting device having a tandem structure.

Charge generation layers CGL1 and CGL2 may each be disposed between adjacent structures among the light-emitting structures ES1, ES2, and ES3. Charge generation layers CGL1 and CGL2 may each independently include a p-type charge generation layer and/or an n-type charge generation layer.

The p-type charge generation layer may include a hole transport host compound, such as NPB. For example, the p-type charge generation layer may include a compound represented by Chemical Formula HT as described herein. The p-type charge generation layer may further include a p-dopant, such as TCNQ or the like.

In some embodiments, the n-type charge generation layer may include at least one of an alkali metal, an alkaline earth metal, a lanthanide metal, a rare earth metal, a transition metal, a post-transition metal, or an alloy thereof.

The n-type charge generation layer may further include, e.g., a metal complex, and the metal complex can include the above-described metal and at least one organic ligand. The organic ligand may include, e.g., hydroxyquinoline, hydroxyisoquinoline, hydroxybenzoquinoline, hydroxyacridine, hydroxyphenanthridine, hydroxyphenyloxazole, hydroxyphenylthiazole, hydroxydiphenyloxadiazole, hydroxydiphenylthiadiazole, hydroxyphenylpyridine, hydroxyphenylbenzimidazole, hydroxyphenylbenzothiazole, bipyridine, phenanthroline, cyclopentadiene, or the like.

The n-type charge generation layer may further include an electron transport host compound. For example, the n-type charge generation layer may include a compound represented by Chemical Formula ET as described herein. In an embodiment, the n-type charge generation layer may include a phenanthroline-containing compound.

For example, a thickness of the n-type charge generation layer and a thickness of the p-type charge generation layer may each independently be from 20 Å to 1000 Å, from 20 Å to 700 Å, or from 30 Å to 500 Å.

The charge generation layers CGL1 and CGL2 may include a first charge generation layer CGL1 disposed between the first light-emitting structure ES1 and the second light-emitting structure ES2, and a second charge generation layer CGL2 disposed between the second light-emitting structure ES2 and the third-light emitting structure ES3.

In example embodiments, the first light-emitting structure ES1, the first charge generation layer CGL1, the second light-emitting structure ES2, the second charge generation layer CGL2, the third light-emitting structure ES3, and the second electrode 150 may be sequentially stacked from a top surface of the first electrode 110.

Colors emitted from the first light-emitting structure ES1, the second light-emitting structure ES2, and the third light-emitting structure ES3 may be the same or different from each other. In some embodiments, the first light-emitting structure ES1, the second light-emitting structure ES2, and the third light-emitting structure ES3 may include a red light-emitting layer, a green light-emitting layer, and a blue light-emitting layer, respectively, and a white light-emitting structure may be implemented through the tandem structure, but is not limited thereto.

In FIG. 5, the 3-stack tandem structure in which three light-emitting structures are stacked is illustrated as an example, but the tandem structure of the light-emitting device is not limited to the structure illustrated in FIG. 5. For example, a 2-stack structure, or a 4-stack structure, a 5-stack structure, or more stacked structure as will be described with reference FIG. 6 may also be implemented.

Referring to FIG. 6, as described with reference to FIG. 5, a tandem structure ED in which the light-emitting structure and a charge generation layer are alternately and repeatedly stacked may be disposed between the first electrode 110 and the second electrode 150.

In example embodiments, first to m^{th} light-emitting structures ES1 to ESm may be sequentially stacked from the top surface of the first electrode 110 with the charge generation layer(s) interposed therebetween. The charge generation layer(s) may include a first charge generation layer CGL1 to an (m−1)th charge generation layer CGLm−1 sequentially stacked from the top surface of the first electrode 110.

As illustrated in FIG. 6, the first light-emitting structure ES1, the first charge generation layer CGL1, the second light-emitting structure ES2, the second charge generation layer CGL2, . . . , an (m−1)th light-emitting structure ESm−1, an (m−1)th charge generation layer CGLm−1, an m^{th} light-emitting structure ESm, and the second electrode 150 may be sequentially stacked from the top surface of the first electrode 110.

In some embodiments, m may be 4, and an intermediate layer of the light-emitting device may have a 4-stack tandem structure, and may include first to fourth light-emitting structures ES1, ES2, ES3, and ES4, and first to third charge generation layers CGL1, CGL2, and CGL3. Colors of light generated from the first to fourth light-emitting structures ES1, ES2, ES3, and ES4 may be the same or different from each other.

In an embodiment, the first to fourth light emitting structures ES1, ES2, ES3, and ES4 may include at least one blue light-emitting structure and at least one green-light emitting structure. In a non-limiting example, the first to third light emitting structures ES1, ES2, and ES3 may correspond to the blue light-emitting structure, and the fourth light emitting structure ES4 may correspond to the green-light emitting structure.

In some embodiments, m may be 5, and an intermediate layer of the light-emitting device may have a 5-stack tandem structure, and may include first to fifth light-emitting structures ES1, ES2, ES3, ES4, and ES5, and first to fourth charge generation layers CGL1, CGL2, CGL3, and CGL4. Colors of light generated from the first to fifth light-emitting structures ES1, ES2, ES3, ES4, and ES5 may be the same or different from each other.

In an embodiment, the first to fifth light-emitting structures ES1, ES2, ES3, ES4, and ES5 may include at least one blue light emitting structure and at least one green light emitting structure. In a non-limiting example, the first to fifth light-emitting structures ES1, ES2, ES3, ES4, and ES5 may include three blue light-emitting structures and two green light-emitting structures. For example, the first, third, and fifth light-emitting structures ES1, ES3, and ES5 may correspond to the blue light-emitting structures, and the second and fourth light-emitting structures ES2 and ES4 may correspond to the green light-emitting structures.

### Electronic Device

The above-described light-emitting device ED may be applied to an electronic device and may be provided as a light-emitting portion or a light-emitting unit of the electronic device.

The electronic device may include the light-emitting device ED including the condensed ring compound of Chemical Formula 1 described herein, thereby providing improved luminous efficiency and life-span properties.

The electronic device may further include, e.g., a functional layer disposed on the light-emitting device, and the functional layer may include a sensor layer, a polarizing layer, a color conversion layer, a color filter layer, or a combination of at least two thereof.

Examples of an electronic device may include a display device, a billboard, a signboard, a light source, a lighting device, a personal computer such as a laptop computer or a desktop computer, a mobile phone, an electronic book, an electronic dictionary, an electronic notebook, a health-care device including a diagnostic device and various sensors, various display parts for transportation means (automobile, aircraft, ship, train, etc.). For example, the electronic device may be one of a flat panel display, a curved display, a computer monitor, a medical monitor, a television, a billboard, a light for indoor or outdoor lighting, a signal lighting, a head-up display, a full or partial transparent display, a flexible display, a rollable display, a foldable display, a stretchable display, a laser printer, a phone, a mobile phone, a tablet, a phablet, a personal information terminal (PDA), a wearable device, a laptop computer, a digital camera, a camcorder, a viewfinder, a micro display, a 3D display, a virtual reality or augmented reality display, a vehicle, a video wall including multiple displays tiled together, a theater or stadium screen, a phototherapy device, or a signage.

In example embodiments, the light-emitting device ED may be applied to an organic light emitting diode (OLED) display device or a quantum dot (QD)-OLED display device.

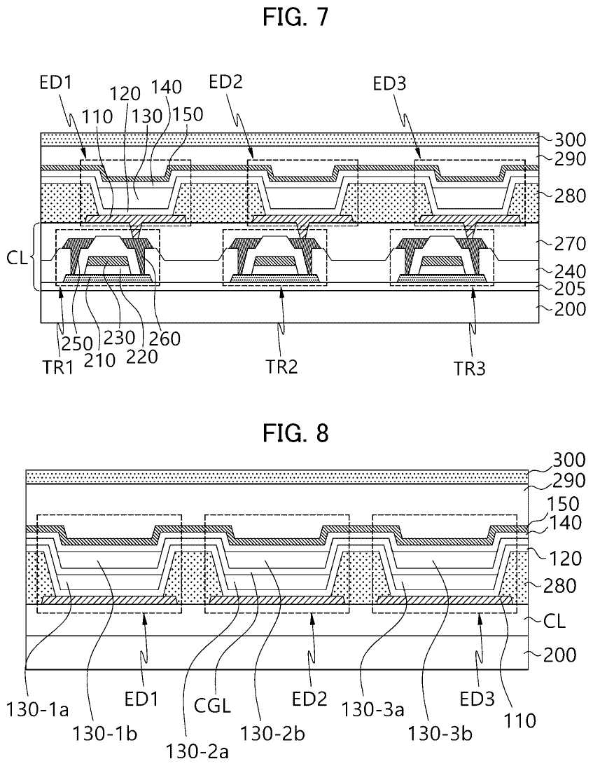

FIG. 7 is a schematic cross-sectional view illustrating a display device in accordance with example embodiments.

Referring to FIG. 7, the display device may include a circuit layer CL disposed on a base substrate 200, and light-emitting devices ED1, ED2, and ED3 disposed on the circuit layer CL.

The base substrate 200 may serve as a supporting substrate or as a back-plane substrate of a display device. The base substrate 200 may be a glass substrate or a plastic substrate.

In some embodiments, the base substrate 200 may include a polymer material having transparent and flexible properties. When the base substrate 200 includes a polymer material, the base substrate 200 may be used in a transparent flexible display device. For example, the base substrate 200 may include a polymer material such as polyimide, polysiloxane, an epoxy resin, an acrylic resin, polyester, or the like. In an embodiment, the base substrate 200 may include a polyimide.

The circuit layer CL may include transistors TR1, TR2, and TR3. The circuit layer CL may include wiring layers and insulating layers that form a thin film transistor array (TFT-Array).

The circuit layer CL may further include a buffer layer 205 on a top surface of the base substrate 200. The buffer layer 205 may block the penetration of moisture through the base substrate 200, and may also block the diffusion of impurities between the base substrate 200 and the structures formed thereon.

The buffer layer 205 may include, for example, silicon oxide, silicon nitride, or silicon oxynitride. The buffer layer 205 may include one of the aforementioned materials, or a combination thereof. In some embodiments, the buffer layer 205 may have a stacked structure that includes a silicon oxide layer and a silicon nitride layer.

The transistors TR1, TR2, and TR3 may be disposed on the buffer layer 205. A first transistor TR1, a second transistor TR2, and a third transistor TR3 may be electrically connected to a first light-emitting device ED1, a second light-emitting device ED2, and a third light-emitting device ED3, respectively.

The transistors TR1, TR2, and TR3 may each include an active layer 210, a gate insulation layer 220, and a gate electrode 230.

The active layer 210 may be disposed on the buffer layer 205, and may be patterned for each pixel. The active layer 210 may include a silicon compound such as amorphous silicon or polysilicon. A p-type dopant or an n-type dopant may be doped in a region of the active layer 210, and the active layer 210 may include a source region, a drain region, and a channel region.

The active layer 210 may include an oxide semiconductor, such as indium gallium zinc oxide (IGZO), zinc tin oxide (ZTO), or indium tin zinc oxide (ITZO).

The gate insulation layer 220 may be formed on the active layer 210, and the gate electrode 230 may be stacked on the gate insulation layer 220. As illustrated in FIG. 7, the gate insulation layer 220 may be patterned to partially cover each active layer 210. Alternatively, the gate insulation layer 220 may extend continuously over multiple pixels or light-emitting regions, and may be provided as a common layer for the first, second, and third transistors TR1, TR2, and TR3.

The gate electrode 230 may overlap the channel region of the active layer 210 in a thickness direction.

An insulating interlayer 240 may be formed on the active layer 210 to cover the gate electrode 230 and the gate insulation layer 220. Connection electrodes 250 and 260 which may be in contact with or electrically connected to the active layer 210 may each be disposed on the insulating interlayer 240.

The connection electrodes 250 and 260 may extend through the insulating interlayer 240 to be in contact with or electrically connected to the active layer 210. When the gate insulation layer 220 is provided as a common layer for multiple light-emitting regions, the connection electrodes 250 and 260 may also extend through the gate insulation layer 220.

The connection electrodes 250 and 260 may include a source electrode 250 that may be in contact with or connected to the source region of the active layer 210, and a drain electrode 260 that may be in contact with or connected to the drain region of the active layer 210.

The gate insulation layer 220 and the insulating interlayer 240 may each independently include silicon oxide, silicon nitride, or silicon oxynitride, and may each have a stacked structure that includes a silicon oxide layer and a silicon nitride layer.

The gate electrode 230 and the connection electrodes 250 and 260 may include a metal such as Ag, Mg, Al, W, Cu, Ni, Cr, Mo, Ti, Pt, Ta, Nd, Sc, an alloy thereof, or a nitride thereof.

A via insulation layer 270 may be formed on the insulating interlayer 240 to cover the connection electrodes 250 and 260.

The via insulation layer 270 may accommodate a via structure electrically connecting the first electrode 110 and the drain electrode 260. The via insulation layer 270 may serve as a planarization layer of the circuit layer CL. In embodiments, the via insulation layer 270 may include an organic material such as polyimide, an epoxy resin, an acrylic resin, polyester, or the like, or a combination thereof.

The light-emitting devices ED1, ED2, and ED3 may be disposed on the via insulation layer 270. For example, as described with reference to FIGS. 1 to 4, the light-emitting devices ED1, ED2, and ED3 may include the first electrode 110, the hole transfer region 120, the emission layer 130, the electron transfer region 140, and the second electrode 150 which are sequentially stacked from the via insulation layer 270.

The first electrode 110 may be electrically connected to the transistors TR1, TR2, and TR3 or the connection electrodes 250 and 260 in the circuit layer CL through the via structure. As illustrated in FIG. 7, the first electrode 110 may be in contact with or connected to the drain electrode 260 to serve as a pixel electrode patterned for each light-emitting region or pixel.

A pixel defining layer 280 may be formed on the via insulation layer 270 to define each light-emitting region or pixel. A blue light-emitting region, a red light-emitting region, and a green light-emitting region may be separated and defined by the pixel defining layer 280, and the light-emitting devices ED1, ED2, and ED3 may respectively correspond to a blue light-emitting device, a red light-emitting device, and a green light-emitting device.

The pixel defining layer 280 may partially cover the first electrode 110 of each light-emitting region.

As illustrated in FIG. 7, the hole transfer region 120 and the electron transfer region 140 may each be provided as a common layer that continuously extends over the pixel defining layer 280 and the first electrodes 110. The emission layer 130 may be formed within each light emitting-region or pixel, and may be separated by the pixel defining layer 280.

In some embodiments, the emission layer 130 may also be provided as a common layer that continuously extends over the light emitting-regions or pixels. In some embodiments, the hole transfer region 120, the emission layer 130, and the electron transfer region 140 may each be patterned and separately formed for each light-emitting region or pixel.

The second electrode 150 may be provided as a common electrode that continuously extends over the light-emitting regions or the pixels.

An encapsulation layer 290 may be disposed on the pixel defining layer 280 and the light-emitting devices ED1, ED2, and ED3 to protect the light-emitting devices ED1, ED2, and ED3 from moisture and/or oxygen. The encapsulation layer 290 may be a thin film encapsulation (TFE) having a single-layered structure or multi-layered structure.

The encapsulation layer 290 may include an inorganic layer that includes silicon nitride (SiNₓ), silicon oxide (SiOₓ), indium tin oxide, indium zinc oxide, or any combination thereof; an organic layer that includes polyethylene terephthalate, polyethylene naphthalate, polycarbonate, polyimide, polyethylene sulfonate, polyoxymethylene, polyarylate, hexamethyldisiloxane, an acrylic resin (e.g., polymethylmethacrylate, polyacrylic acid, or the like), an epoxy resin (e.g., an aliphatic glycidyl ether (AGE)), or any combination thereof; or a combination of the inorganic layer and the organic layer.

The display device may further include a functional layer 300 disposed on the encapsulation layer 290. The functional layer 300 may include a sensor layer such as a touch sensor layer, an optical layer such as a polarizing layer, a color conversion layer, a color filter layer, a window film, or any combination thereof.

FIG. 8 is a schematic cross-sectional view illustrating a display device in accordance with example embodiments.

Referring to FIG. 8, each of the light-emitting devices ED1, ED2, and ED3 may have a tandem structure, e.g., a 2-stack tandem structure.

In some embodiments, the hole transfer region 120 and the electron transfer region 140 may be continuously and commonly formed and included in an intermediate layer of each light-emitting structure. Additionally, a charge generation layer CGL may continuously extend across a plurality of pixels and may be commonly included in the intermediate layer of each light-emitting structure.

The first light-emitting device ED1 may include a first lower emission layer 130-1a disposed between the hole transfer region 120 and the charge generation layer CGL, and a first upper emission layer 130-1b disposed between the charge generation layer CGL and the electron transfer region 140.

The second light-emitting device ED2 may include a second lower emission layer 130-2a disposed between the hole transfer region 120 and the charge generation layer CGL, and a second upper emission layer 130-2b disposed between the charge generation layer CGL and the electron transfer region 140.

The third light-emitting device ED3 may include a third lower emission layer 130-3a disposed between the hole transfer region 120 and the charge generation layer CGL, and a third upper emission layer 130-3b disposed between the charge generation layer CGL and the electron transfer region 140.

The lower and upper emission layers included in each light-emitting structure may generate light of the same color. In an embodiment, each of the first lower emission layer 130-1a and the first upper emission layer 130-1b included in the first light-emitting device ED1 may correspond to a red emission layer. Each of the second lower emission layer 130-2a and the second upper emission layer 130-2b included in the second light-emitting device ED2 may correspond to a green emission layer. Each of the third lower emission layer 130-3a and the third upper emission layer 130-3b included in the third light-emitting device ED3 may correspond to a blue emission layer.

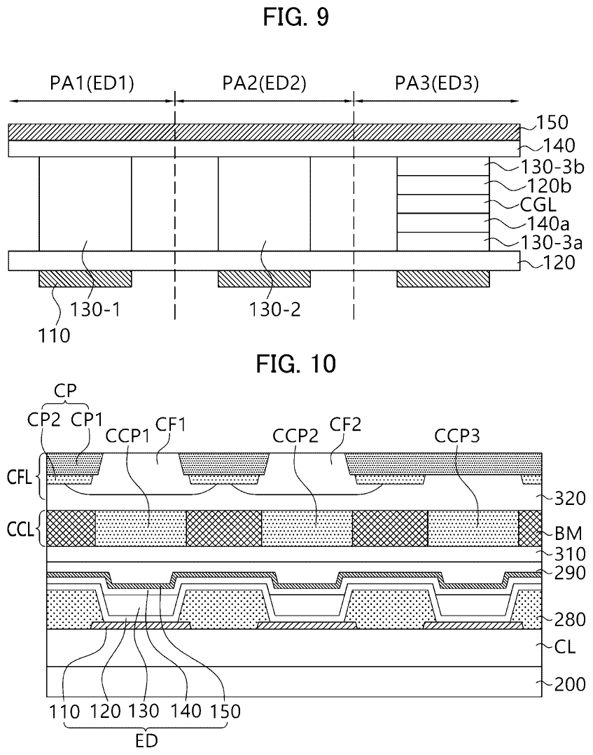

FIG. 9 is a schematic cross-sectional view illustrating a stack construction of light-emitting structure in a display device in accordance with example embodiments. For convenience of illustration and description, illustration of the circuit layer, the base substrate, the pixel defining layer, etc., is omitted from FIG. 9, and a shape of each layer or element in the light-emitting structure is briefly shown as a rectangle.

Referring to FIG. 9, at least one of the light-emitting devices ED1, ED2, or ED3 or pixel areas PA1, PA2, or PA3 may have a tandem structure including a plurality of emission layers, and at least one of the remainder may have a single emission layer structure.

In some embodiments, one of the light-emitting devices ED1, ED2, or ED3 or the pixel areas PA1, PA2 or PA3 may have a tandem structure, and the remainder may have a single emission layer structure.

As illustrated in FIG. 9, the first light-emitting device ED1, the second light-emitting device ED2, and the third light-emitting device ED3 may be included in the first pixel area PA1, the second pixel area PA2, and the third pixel area PA3, respectively. In some embodiments, the first pixel area PA1, the second pixel area PA2, and the third pixel area PA3 may correspond to a red pixel area, a green pixel area, and a blue pixel area, respectively.

The hole transfer region 120, the electron transfer region 140, and the second electrode 150 may each be provided as a common layer continuously extending over the first pixel area PA1, the second pixel area PA2, and the third pixel area PA3.

The first-light emitting device ED1 included in the first pixel area PA1 may include a first emission layer 130-1, and the second light-emitting device ED2 included in the second pixel area PA2 may include a second emission layer 130-2. Each of the first emission layer 130-1 and the second emission layer 130-2 may be a single-layered emission layer.

The third light-emitting device ED3 included in the third pixel area PA3 may have, e.g., a 2-stack tandem structure. The third light-emitting device ED3 may include a third lower emission layer 130-3a and a third upper emission layer 130-3b separated with the charge generation layer CGL interposed therebetween. Each of the third lower emission layer 130-3a and the third upper emission layer 130-3b may correspond to a blue emission layer.

A lower electron transfer region 140a may be disposed between the charge generation layer CGL and the third lower emission layer 130-3a. An upper hole transfer region 120b may be disposed between the charge generation layer CGL and the third upper emission layer 130-3b.

Accordingly, a tandem light-emitting structure in which the first electrode 110, the hole transfer region 120, the third lower emission layer 130-3a, the lower electron transfer region 140a, the charge generation layer CGL, the upper hole transfer region 120b, the third upper emission layer 130-3b, the electron transfer region 140, and the second electrode 150 are sequentially stacked may be disposed in the third pixel area PA3.

FIG. 10 is a schematic cross-sectional view illustrating a display device in accordance with example embodiments.

FIG. 10 illustrates a display device having a QD-OLED structure according to embodiments. Detailed descriptions regarding elements and structures that are the same as or substantially similar to those described with reference to FIG. 7 will not be repeated here.

Referring to FIG. 10, the pixel defining layer 280 and the light-emitting device ED may be disposed on the circuit layer CL, as described above with reference to FIG. 7. In example embodiments, each pixel may emit light of the same wavelength region. In an embodiment, each light-emitting device ED may emit a blue light.

In some embodiments, each light-emitting region may include the light-emitting device having the tandem structure, as described above with respect to FIG. 5. In this case, the intermediate layer of each light-emitting device ED may be provided as a common layer that continuously extends over a plurality of the light-emitting regions.

A color control layer CCL may be disposed on the encapsulation layer 290, and the color control layer CCL may include color control portions CCP1, CCP2, and CCP3.

The color control portions CCP1, CCP2, and CCP3 may each include a light transformer such as a quantum dot or a phosphor. In each of the color control portions CCP1, CCP2, and CCP3, the light transformer may convert a wavelength of a provided light and emit a resulting light.

The color control portions CCP1, CCP2, and CCP3 may be separated or spaced apart from each other by a bank BM. The bank BM may substantially overlap the pixel defining layer 280, and the color control portions CCP1, CCP2, and CCP3 may substantially overlap each of the emission layers 130.

The color control layer CCL may include a first color control portion CCP1 including a first quantum dot that converts a first color light provided from the light-emitting device ED into a second color light, a second color control portion CCP2 including a second quantum dot that converts the first color light into a third color light, and a third color control portion CCP3 that transmits the first color light.

In some embodiments, the first color light, the second color light, and the third color light may be a blue light, a red light, and a green light, respectively. The first quantum dot and the second quantum dot may respectively be a red quantum dot and a green quantum dot.

The color control portions CCP1, CCP2, and CCP3 may each further include a scattering material such as inorganic particles. For example, the third color control portion CCP3 may not include quantum dots and may include the scattering material. The scattering material may include TiO₂, ZnO, Al₂O₃, SiO₂, hollow silica, or the like. The scattering material may be one of the aforementioned materials or a combination thereof.

The color control portions CCP1, CCP2, and CCP3 may each further include a binder resin that disperses the quantum dot and the scattering material. The binder resin may include an acrylic resin, a urethane resin, a silicone resin, an epoxy resin, or the like.

A color filter layer CFL that includes color filters CF1 and CF2 and a light-shielding portion CP may be disposed on the color control layer CCL.

The color filter layer CFL may include a first filter CF1 that transmits the second color light, a second filter CF2 that transmits the third color light, and a third filter that transmits the first color light. For example, the first filter CF1 may be a red filter, the second filter CF2 may be a green filter, and the third filter may be a blue filter.

The color filters CF1 and CF2 may each include a photosensitive binder resin and a colorant including a pigment and/or a dye. The first filter CF1 may include a red pigment or dye, and the second filter CF2 may include a green pigment or dye.

The light-shielding portion CP may be disposed between the color filters. In some embodiments, the light-shielding portion may include a first light-shielding portion CP1 and a second light-shielding portion CP2 that includes colorants of different colors.

In some embodiments, the first light-shielding portion CP1 may include a blue colorant, and the second light-shielding portion CP2 may include a red colorant or a black colorant. In an embodiment, in the blue light-emitting region, a portion of the first light-shielding portion CP1 may be provided as a blue color filter and may be exposed between the second light-shielding portions CP2, so that an additional color filter (e.g., the third filter) may be omitted.

A first barrier layer 310 may be disposed between the color control layer CCL and the light-emitting device ED (or the encapsulation layer 290). A second barrier layer 320 may be disposed between the color control layer CCL and the color filter layer CFL.

The barrier layers 310 and 320 may each include at least one inorganic layer. For example, the barrier layers 310 and 320 may each independently include silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, or the like.

In an embodiment, the barrier layers 310 and 320 may each have a multi-layered structure that further includes an organic layer.

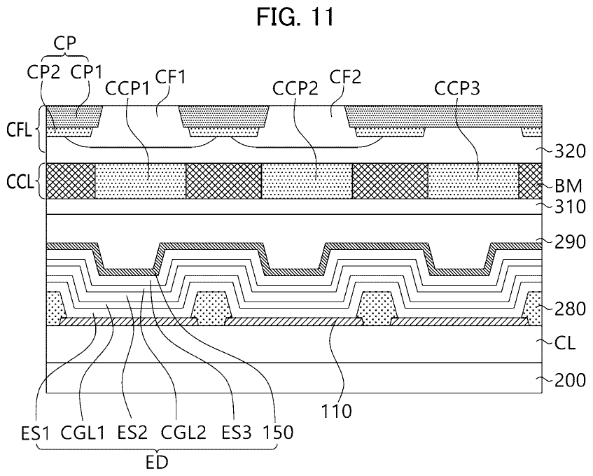

FIG. 11 is a schematic cross-sectional view illustrating a display device in accordance with example embodiments. Detailed descriptions of elements and structures substantially the same as or similar to those described with reference to FIG. 10 are omitted herein.

Referring to FIG. 11, the light-emitting device ED corresponding to the color control portions CCP1, CCP2, and CCP3 may be disposed on the first electrode 110 serving as the pixel electrode, and the light-emitting device ED may have a tandem structure.

In some embodiments, as described with reference to FIG. 5, the first light-emitting structure ES1, the first charge generation layer CGL1, the second light-emitting structure ES2, the second charge generation layer CGL2, and the third light-emitting structure ES3 may be sequentially stacked between the first electrode 110 and the second electrode 150. The first light-emitting structure ES1, the first charge generation layer CGL1, the second light-emitting structure ES2, the second charge generation layer CGL2, and the third light-emitting structure ES3 may be continuously and commonly formed in a plurality of pixel areas or light-emitting regions.

In an embodiment, the first light-emitting structure ES1, the second light-emitting structure ES2, and the third light-emitting structure ES3 may generate different color lights, and the light-emitting device ED may generate a white light. In an embodiment, the first light-emitting structure ES1, the second light-emitting structure ES2, and the third light-emitting structure ES3 may all generate blue lights.

In some embodiments, as described with reference to FIG. 6, the light-emitting device ED may include a tandem structure of 4-stack, 5-stack, or more of the stacked number.

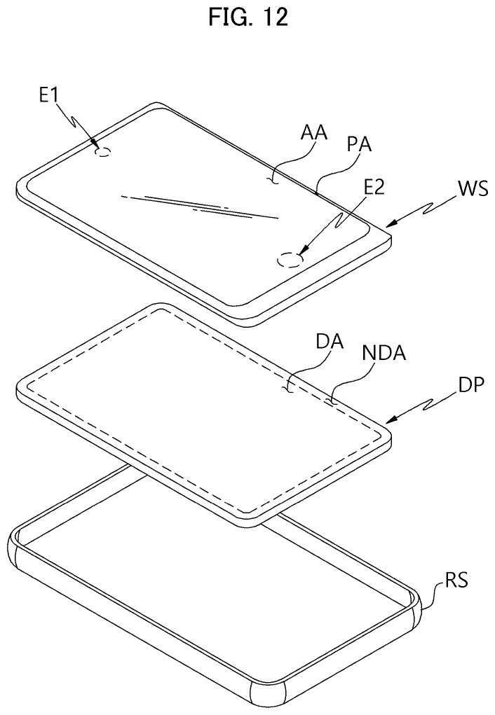

FIG. 12 is a schematic exploded perspective view illustrating an electronic device in accordance with example embodiments.

According to example embodiments, the electronic device may be implemented in the form of a mobile phone (smart phone), a tablet, a PC, or the like, including the above-described display device.

Referring to FIG. 12, the electronic device may include a window structure WS, a display panel DP, and a rear structure RS.

The window structure WS may provide an external display surface recognized by a user, such as a viewing surface of a mobile phone, and may include a transparent material film. For example, the window structure WS may include glass (e.g., ultra-thin glass (UTG), a hard coating film, a plastic film, or the like.

An outer surface of the window structure WS may include an active area AA and a peripheral area PA. The active area AA may provide a surface from which an image of the display device DD is substantially displayed and to which a user's touch/command is input. The peripheral area PA may substantially correspond to a bezel area of the display device.

The display panel DP may include the above-described display device and may have a display area DA and a non-display area NDA. The display area DA of the display panel DP may substantially correspond to or overlap the active area AA of the window structure WS. The non-display area NDA of the display panel DP may substantially correspond to or overlap the peripheral area PA of the window structure WS.

In some embodiments, functional device areas E1 and E2 may be included in the active area AA of the window structure WS. For example, a first functional device area E1 may be included at one end portion of the active area AA and may be implemented, e.g., in the form of a camera hole. The second functional device area E2 may serve as a fingerprint sensing area.

For example, a sensor structure for a touch sensing or a fingerprint sensing may be disposed in the display panel DP or between the window structure WS and the display panel DP.

The rear structure RS may serve as a frame structure or a housing of the display device or the electronic device. A cover panel may be disposed between the rear structure RS and the display panel DP.

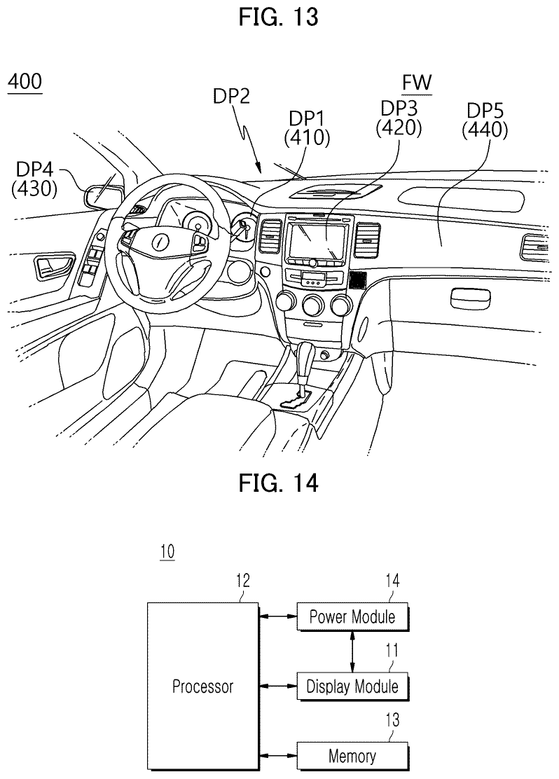

FIG. 13 is a schematic cross-sectional view illustrating an electronic device in accordance with an example embodiment.

The electronic device may be installed in, embedded in, attached to, or integrated with a vehicle 400. However, the vehicle 400 is not limited to the embodiment illustrated in FIG. 13 Further examples of the vehicle 400 may include a transportation means such as a three-wheeled or four-wheeled vehicle, a construction machine, a two-wheeled vehicle, a motor vehicle, a bicycle, a train, etc. Other examples of the vehicle 400 may include an electric vehicle, a hybrid vehicle, etc.

Referring to FIG. 13, at least one of first to fifth display devices DP1, DP2, DP3, DP4, and DP5 may be applied to the vehicle 400.

In example embodiments, the first display device DP1 may be disposed in a cluster area 410. Driving information such as a driving distance and speed, and various warning lights may be displayed in the cluster area 410.

The second display device DP2 may be disposed on a front window FW of the vehicle 400. For example, the second display device DP2 may be installed as a head-up display (HUD).

The third display device DP3 may be disposed on a center fascia 420 of the vehicle 400. In the center fascia 420, a button or a switch for controlling an image display or a music player, an air conditioner, a heater, etc., may be displayed, and vehicle information may be displayed thereon.

The fourth display device DP4 may be applied to side mirrors 430 of the vehicle 400. A side mirror 430 may be installed at each of both sides of an exterior of the vehicle 400, and the fourth display device DP4 may be applied to at least one of the side mirrors 430 installed at each of the both sides.

The fifth display device DP5 may be disposed on a passenger seat dashboard 440. Information/image identical to or different from information/image displayed on the cluster area 410 and/or the center fascia 420 may be displayed at the passenger seat dashboard 440.

The electronic device may include, e.g., a flat panel display, a curved display, a computer monitor, a medical monitor, a television, a billboard, a light for indoor or outdoor lighting, a signal light, a head-up display, a full or partial transparent display, a flexible display, a rollable display, a foldable display, a stretchable display, a laser printer, a phone, a mobile phone, a tablet, a phablet, a personal digital assistant (PDA), a wearable device, a laptop computer, a digital camera, a camcorder, a viewfinder, a micro display, a 3D display, a virtual reality or augmented reality display, a vehicle, a video wall including multiple displays tiled together, a theater or stadium screen, a phototherapy device, or a signage.

The display device according to the embodiments of the present disclosure may be applied to various electronic devices. The electronic device according to an embodiment includes the above-described display device, and may further include a module or device having another additional function in addition to the display device.

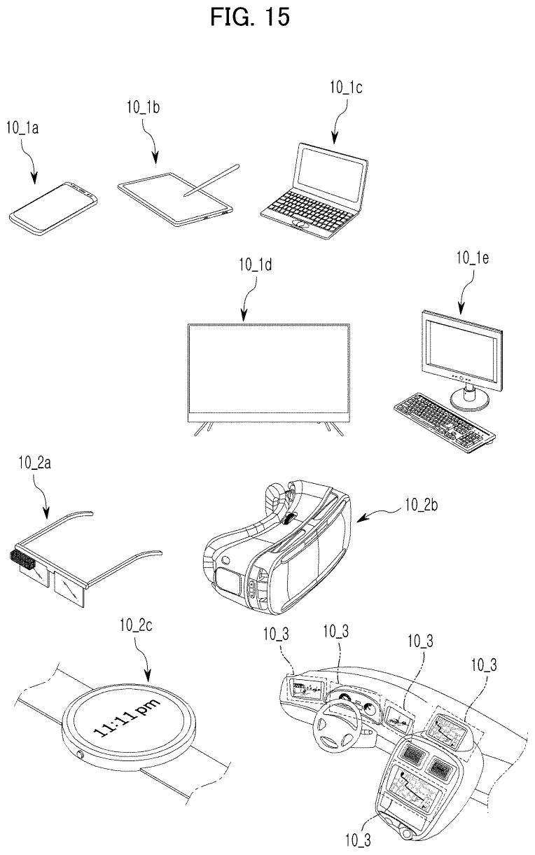

FIG. 14 is a block diagram of an electronic device in accordance with an embodiment.

Referring to FIG. 14, an electronic device 10 according to an embodiment may include a display module 11, a processor 12, a memory 13, and a power module 14.

The processor 12 may include a central processing unit (CPU), an application processor (AP), a graphic processing unit (GPU), a communication processor (CP), an image signal processor (ISP), and/or a controller.

Data information for an operation of the processor 12 or the display module 11 may be stored in the memory 13. When the processor 12 executes an application stored in the memory 13, an image data signal and/or an input control signal may be transmitted to the display module 11, and the display module 11 may process the received signal and output image information through a display screen.

The power module 14 may include a power supply module such as a power adapter or a battery device, and a power conversion module that converts a power supplied by the power supply module to generate power required for the operation of the electronic device 10.

At least one of the components of the electronic device 10 as described above may be included in the display device according to the above-described embodiments. Additionally, some of the individual modules functionally included in one module may be included in the display device, and others may be provided separately from the display device. For example, the display module 11 may include the display device, and the processor 12, the memory 13 and the power module 14 may be provided in the form of another device in the electronic device 10 different from the display device.

FIG. 15 is a schematic diagram of an electronic device in accordance with various embodiments.

Referring to FIG. 15, non-limiting examples of various electronic devices to which the display device according to the above-described embodiments is applied include an electronic device for displaying an image such as a smartphone 10_1a, a tablet PC 10_1b, a laptop 101c, a TV 10_1d, a desk monitor 10_1e, or the like; a wearable electronic device including a display module such as smart glasses 10_2a, a head mounted display 10_2b, a smart watch 10_2c, or the like; a vehicle electronic device 10_3 including a display module such as a center information display (CID) disposed at a vehicle instrument panel, a center fascia, a dashboard, etc., a room mirror display, a head-up display, or the like. The electronic device may include a virtual reality glass or an augmented reality glass.

Hereinafter, an organometallic compound according to an embodiment will be described in further detail with reference to the Examples and the Comparative Examples. The Examples are provided to assist in understanding the disclosure, but they are provided as non-limiting examples, and the scope of the disclosure is not limited thereto. It will be clear to those skilled in the art that various changes and modifications to disclosed examples can be made within the scope of the disclosure.

### EXAMPLES

### Synthesis Example 1: Synthesis of Compound 10

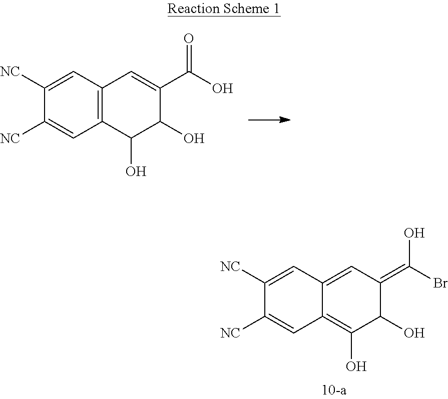 [SMILES: N#Cc1cc2c(cc1C#N)C(O)C(O)C(C(=O)O)=C2 ; N#Cc1cc2c(cc1C#N)=C(O)C(O)/C(=C(/O)Br)C=2]

In an inert gas environment, 6,7-dicyano-3,4-dihydroxy-3,4-dihydronaphthalene-2-carboxylic acid (10.00 grams (g), 39 millimoles (mmol)) was added to deionized (DI) water, and PBr₃ (12.68 g, 47 mmol) was added to the stirred solution. The reaction proceeded while heating to 120° C. to obtain a compound 10-a (11.6 g) (yield: 93.1%).

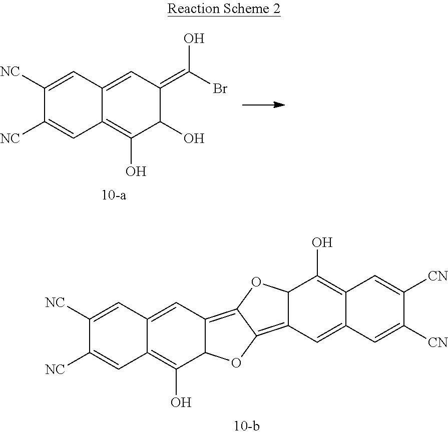 [SMILES: N#Cc1cc2c(cc1C#N)=C(O)C(O)/C(=C(/O)Br)C=2 ; N#Cc1cc2c(cc1C#N)=C(O)C1OC3=C4C=c5cc(C#N)c(C#N)cc5=C(O)C4OC3=C1C=2]

In an inert gas environment, phenyl boronic acid (4.59 g, 38 mmol), sodium carbonate (3.04 g, 38 mmol) and a palladium catalyst (1 mol %) in 1,3,5-triphenylbenzene-1,2-bis(diphenylphosphino)ethane-dimethoxymethane were dissolved in ethanol/water (2:3 volume ratio) to prepare a solution.

The compound 10-a (10.00 g, 31 mmol) was added to the solution and stirred at 80° C. for 2 hours. The solution was cooled and extracted with dichloromethane (DCM). The obtained product and Nafion-H catalyst were added to toluene, stirred, and heated at reflux for 36 hours to obtain a compound 10-b (5.6 g) (yield: 80.8%).

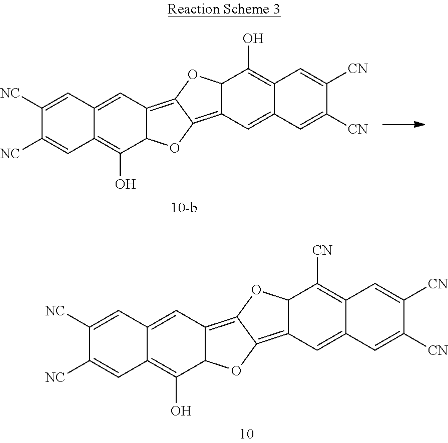 [SMILES: N#Cc1cc2c(cc1C#N)=C(O)C1OC3=C4C=c5cc(C#N)c(C#N)cc5=C(O)C4OC3=C1C=2 ; N#CC1=c2cc(C#N)c(C#N)cc2=CC2=C3OC4C(=C3OC21)C=c1cc(C#N)c(C#N)cc1=C4O]

The compound 10-b (5.50 g, 12 mmol) and cyanogen bromide (2.90 g, 27 mmol) were dissolved in a pentane/water (volume ratio 5:2) solvent, and cooled to 0° C. The solution was stirred for 10 minutes, triethylamine (4 mL) was added dropwise. The mixture was stirred at 0° C. for 1 hour and filtered to obtain a compound 10 (4.9 g) (yield: 85.6%).

### Synthesis Example 2: Synthesis of Compound 8

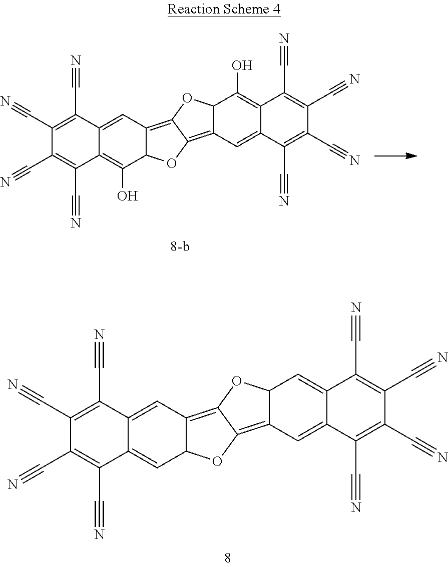 [SMILES: N#Cc1c(C#N)c(C#N)c2c(c1C#N)=CC1=C3OC4C(=C3OC1C=2O)C=c1c(C#N)c(C#N)c(C#N)c(C#N)c1=C4O ; N#Cc1c(C#N)c(C#N)c2c(c1C#N)=CC1=C3OC4C=c5c(C#N)c(C#N)c(C#N)c(C#N)c5=CC4=C3OC1C=2]

The same process as that of the synthetic process for the compound 10-b in Synthesis Example 1 was performed except that 5,6,7,8-tetracyano-3,4-dihydroxy-3,4-dihydronaphthalene-2-carboxylic acid (10.00 g, 33 mmol) was used instead of 6,7-dicyano-3,4-dihydroxy-3,4-dihydronaphthalene-2-carboxylic acid in the synthesis of compound 10-a to obtain a compound 8-b (6.7 g) (yield: 88.3%).

The compound 8-b (6.50 g, 12 mmol) and 50 mg of a CoMo nanosulfide catalyst were added to 50 ml of a decalin solvent and purged with nitrogen for 2 hours to remove air. The reaction vessel was heated to 300° C., and a hydrogen gas was added at 4.0 MPa and stirred at a high speed to obtain a compound 8 (4.9 g) (yield: 80.1%).

### Synthesis Example 3: Synthesis of Compound 13

The same process as that of Reaction Scheme 3 in Synthesis Example 1 was performed except that the compound 8-b (10.00 g, 18 mmol) and 4-bromobenzonitrile (7.38 g, 41 mmol) were used instead of the compound 10-b and cyanogen bromide to obtain a compound 13 (8.5 g) (yield: 64.7%).

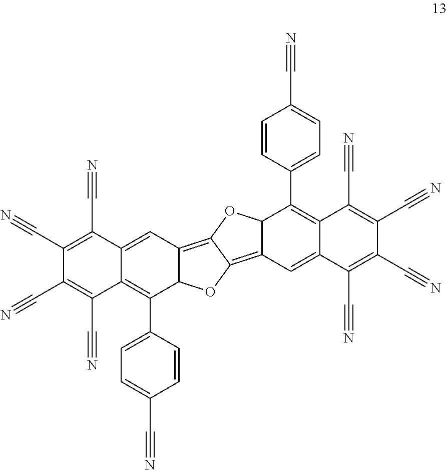 [SMILES: N#Cc1ccc(C2=c3c(C#N)c(C#N)c(C#N)c(C#N)c3=CC3=C4OC5C(=C4OC32)C=c2c(C#N)c(C#N)c(C#N)c(C#N)c2=C5c2ccc(C#N)cc2)cc1]

### Synthesis Example 4: Synthesis of Compound 16

The same process as that of the synthetic process for the compound 10-b in Synthesis Example 1 was performed except that 5,6,7,8-tetracyano-3,4-dihydroxy-3,4-dihydronaphthalene-2-carbothioic O-acid (10.00 g, 31 mmol) was used instead of 6,7-dicyano-3,4-dihydroxy-3,4-dihydronaphthalene-2-carboxylic acid in the synthesis of compound 10-a to obtain an intermediate compound (16-b) (7.50 g).

Thereafter, the same process as that of Reaction Scheme 3 in Synthesis Example 1 was performed except that the intermediate compound (16-b) (7.50 g, 13 mmol) and 4-bromobenzonitrile (5.41 g, 30 mmol) were used instead of the compound 10-b and cyanogen bromide to obtain a compound 16 (6.7 g) (yield: 58.0%).

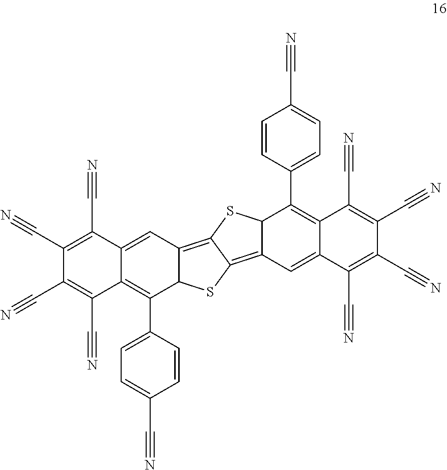 [SMILES: N#Cc1ccc(C2=c3c(C#N)c(C#N)c(C#N)c(C#N)c3=CC3=C4SC5C(=C4SC32)C=c2c(C#N)c(C#N)c(C#N)c(C#N)c2=C5c2ccc(C#N)cc2)cc1]

### Synthesis Example 5: Synthesis of Compound 17

The same process as that of Synthesis Example 4 was performed except that (E)-7-hydroxy-8-(hydroxymethylene)-7,8-dihydronaphthalene-1,2,3,4,5,6-hexacarbonitrile (10.00 g, 31 mmol) was used instead of 5,6,7,8-tetracyano-3,4-dihydroxy-3,4-dihydronaphthalene-2-carbothioic O-acid in the synthesis of the intermediate compound 16-b in Synthesis Example 4 to obtain a compound 17 (3.8 g) (yield: 40.4%).

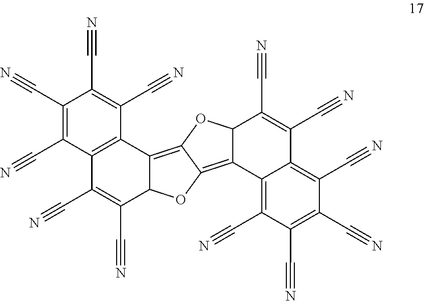

¹H NMR and MS/FAB results of the compounds synthesized in the above Synthesis Examples 1 to 5 are shown in Table 1. For the ¹H NMR data, the values are reported as chemical shifts (□□□ppm), wherein s is singlet, and d is doublet.

TABLE 1

| | | MS/FAB | |
|---|---|---|---|
| | | Found | |
| | ¹H NMR (CDCl₃, 500 MHz) | [M + 1] | calc. |
| Synthetic | 8.05 (s, 4H), 6.46 (s, 2H), 4.86 (s, 2H) | 459.1 | 460.07 |
| Example 1 | | | |
| Synthetic | 6.46 (s, 2H), 5.86 (d, 2H), 4.86 (d, 2H) | 509.2 | 510.06 |
| Example 2 | | | |
| Synthetic | 7.87 (d, 4H), 7.52 (d, 4H), 6.46 (s, | 711.3 | 712.11 |
| Example 3 | 2H), 4.86 (s, 2H) | | |
| Synthetic | 7.87 (d, 4H), 7.52 (d, 4H), 6.46 (s, | 743.1 | 744.07 |
| Example 4 | 2H), 3.90 (s, 2H) | | |
| Synthetic | 4.86 (s, 2H) | 609.3 | 610.04 |
| Example 5 | | | |

### Fabrication of Light-Emitting Device

As the first electrode, a glass substrate (Corning product) on which a 15 ohms per square centimeter (Ω/cm²) (1,300 Å) ITO electrode was formed was cut into a size of 50 millimeters (mm)×50 mm×0.7 mm, and the cut substrate was ultrasonically cleaned for 5 minutes using isopropyl alcohol and DI water. The ultrasonically cleaned substrate was irradiated with an ultraviolet ray for 30 minutes and exposed to ozone, and then mounted on a vacuum deposition device.

Thereafter, a p-dopant compound and HT-15 were vacuum-deposited in a weight ratio of 3:97 on the anode to form a hole injection layer having a thickness of 100 Å. HT-16 was vacuum-deposited on the hole injection layer to form a hole transport layer having a thickness of 1,250 Å. PH-14, PH-15 and PD1-15 were vacuum-deposited on the hole transport layer in a weight ratio of 45:45:10 to form an emission layer having a thickness of 300 Å.

ET-14 was vacuum-deposited on the emission layer to form a hole blocking layer having a thickness of 50 Å. ET-18 and LiQ were vacuum-deposited on the hole blocking layer in a weight ratio of 5:5 to form an electron transport layer having a thickness of 310 Å.

Yb was vacuum-deposited on the electron transport layer to form an electron injection layer having a thickness of 15 Å. Ag and Mg were vacuum-deposited on the electron injection layer in a weight ratio of 5:5 to form a cathode having a thickness of 1,000 Å.

The above-mentioned compounds used in the fabrication of the light-emitting device are shown below. Sublimation-purified compounds from commercially available products were used.

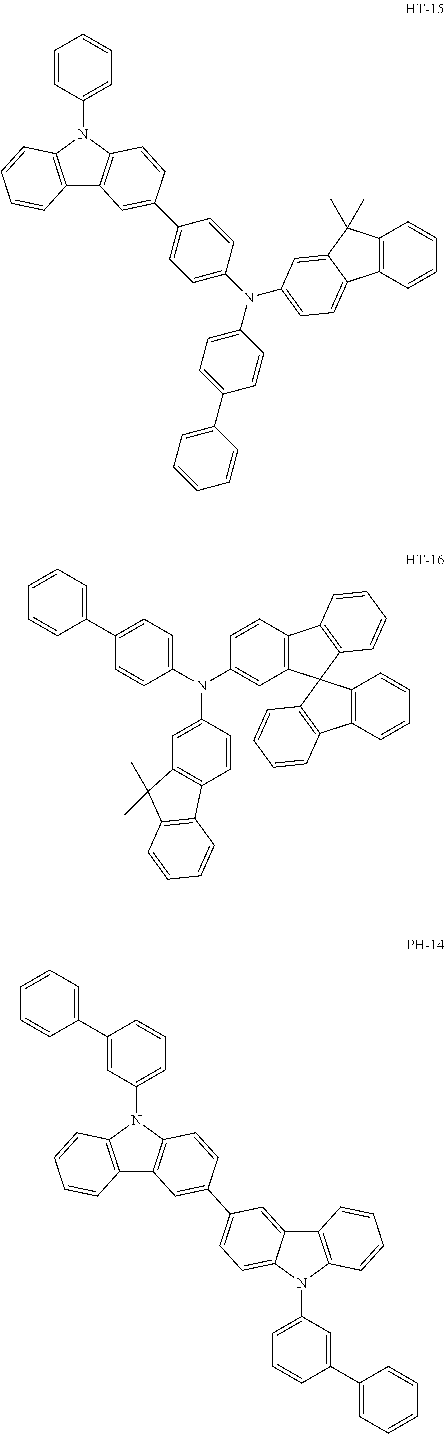 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccc(-c4ccccc4)cc3)c3ccc4c(c3)C3(c5ccccc5-c5ccccc53)c3ccccc3-4)cc21 ; c1ccc(-c2cccc(-n3c4ccccc4c4cc(-c5ccc6c(c5)c5ccccc5n6-c5cccc(-c6ccccc6)c5)ccc43)c2)cc1]

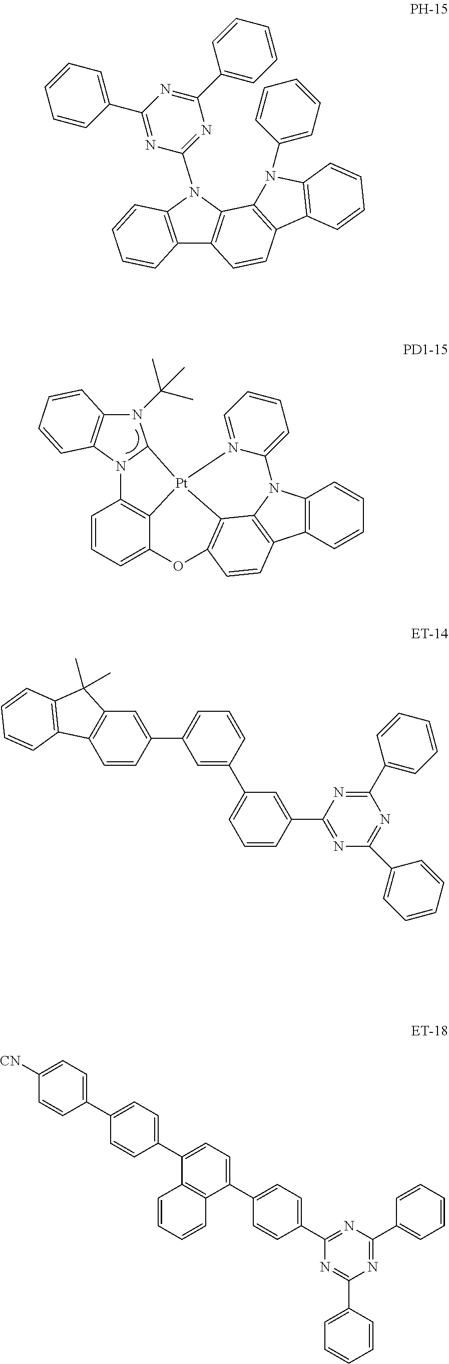

The p-dopant compounds used in the fabrication of the light-emitting device are shown in Table 2 below.

TABLE 2

| | compound |
|---|---|
| Example 1 | 10 (Synthesis Example 1) |
| Example 2 | 8 (Synthesis Example 2) |
| Example 3 | 13 (Synthesis Example 3) |
| Example 4 | 16 (Synthesis Example 4) |
| Example 5 | 17 (Synthesis Example 5) |
| Comparative | 101 |
| Example 1 | |
| Comparative | 102 |
| Example 2 | |
| Comparative | 103 |
| Example 3 | |
| Comparative | 104 |
| Example 4 | |
| Comparative | 105 |
| Example 5 | |
| Comparative | 106 |
| Example 6 | |

The compounds of Comparative Examples are shown below. Sublimation-purified compounds from commercially available products were used.

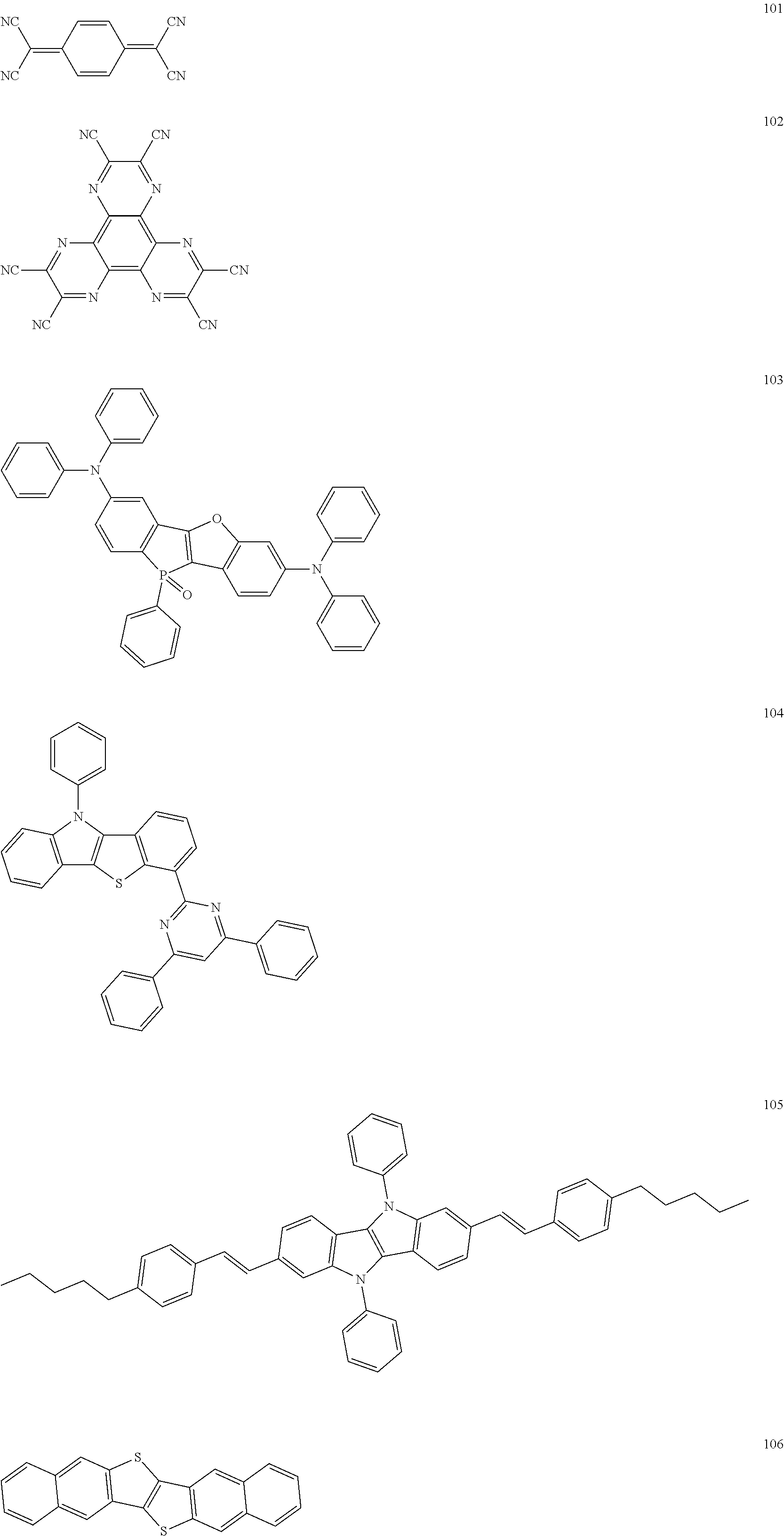 [SMILES: N#CC(C#N)=c1ccc(=C(C#N)C#N)cc1 ; N#Cc1nc2c3nc(C#N)c(C#N)nc3c3nc(C#N)c(C#N)nc3c2nc1C#N ; O=P1(c2ccccc2)c2ccc(N(c3ccccc3)c3ccccc3)cc2-c2oc3cc(N(c4ccccc4)c4ccccc4)ccc3c21 ; c1ccc(-c2cc(-c3ccccc3)nc(-c3cccc4c3sc3c5ccccc5n(-c5ccccc5)c43)n2)cc1 ; CCCCCc1ccc(/C=C/c2ccc3c(c2)n(-c2ccccc2)c2c4ccc(/C=C/c5ccc(CCCCC)cc5)cc4n(-c4ccccc4)c32)cc1 ; c1ccc2cc3c(cc2c1)sc1c2cc4ccccc4cc2sc31]

### EXPERIMENTAL EXAMPLE

### Experimental Example 1: Evaluation on Properties of Condensed Ring Compounds

Properties of the compounds of Examples and Comparative Examples were evaluated as follows. The evaluation results are shown in Table 3.

### (1) HOMO Energy Level Evaluation

Using cyclic voltammetry (CV) (electrolyte: 0.1 M Bu₄NPF₆/solvent: DMF (dimethylformamide)/electrode: 3-electrode system (working electrode: GC (glassy carbon), reference electrode: Ag/AgCl, auxiliary electrode: Pt)), a potential (V)-current (A) graph of each compound was obtained. Thereafter, a HOMO energy level of each compound was calculated from an oxidation onset of the graph.

### (2) LUMO Energy Level Evaluation

Cyclic voltammetry (CV) (electrolyte: 0.1 M Bu₄NPF₆/solvent: DMF (dimethylformamide)/electrode: 3-electrode system (working electrode: GC, reference electrode: Ag/AgCl, auxiliary electrode: Pt)) was used to obtain a potential (V)-current (A) graph of each compound. Thereafter, a LUMO energy level of each compound was calculated from an reduction onset of the graph.

### (3) Hole Mobility and Electron Mobility Evaluation

A hole mobility was evaluated using a space-charge-limited current (SCLC) method described in the literature “Hole mobility of N,N′-bis(napthalen-1-yl)-N,N′-bis(phenyl)benzidine investigated by using space-charge-limited currents, ‘Appl. Phys. Lett. 90, 203512 (2007)”.

### (4) Glass Transition Temperature Evaluation

A glass transition temperature (Tg) was measured by a DSC (Differential Scanning Calorimetry) analysis. Specifically, 5 mg of a sample was heated from room temperature to 300° C. at a ramping rate of 10° C./min, cooled from 300° C. to 25° C., and then heated to 300° C. again. The glass transition temperature was obtained from an inflection point of the graph during the second heating.

TABLE 3

| | | HOMO | LUMO | Hole mobility | Electron mobility | T_{g} |
|---|---|---|---|---|---|---|
| | Compound | (eV) | (eV) | (cm²/Vs) | (cm²/Vs) | (° C.) |
| Example 1 | 10 | −6.59 | −5.09 | 2.17E−02 | 2.46E−02 | 138.7 |
| Example 2 | 8 | −6.83 | −5.33 | 8.63E−03 | 2.80E−02 | 132.5 |
| Example 3 | 13 | −6.69 | −5.38 | 1.51E−03 | 4.13E−03 | 157.9 |
| Example 4 | 16 | −6.57 | −5.40 | 1.59E−03 | 4.10E−03 | 161.4 |
| Example 5 | 17 | −8.18 | −5.22 | 1.49E−03 | 7.78E−03 | 151.6 |
| Comparative | 101 | −7.58 | −5.02 | 8.52E−02 | 6.53E−02 | 101.8 |
| Example 1 | | | | | | |
| Comparative | 102 | −9.05 | −4.81 | 1.69E−03 | 5.01E−03 | 118.6 |
| Example 2 | | | | | | |
| Comparative | 103 | −5.14 | −1.84 | 4.23E−03 | 1.37E−02 | 127.8 |
| Example 3 | | | | | | |
| Comparative | 104 | −5.24 | −1.91 | 1.09E−03 | 6.74E−04 | 100.5 |
| Example 4 | | | | | | |
| Comparative | 105 | −4.82 | −1.79 | 3.27E−02 | 4.25E−03 | 68.9 |
| Example 5 | | | | | | |
| Comparative | 106 | −5.41 | −2.07 | 9.27E−03 | 3.47E−04 | 95.7 |
| Example 6 | | | | | | |

Referring to Table 3, in the condensed ring compounds according to the Examples, the HOMO energy level and the LUMO energy level were appropriately controlled to improve the hole mobility and electron mobility. Accordingly, the condensed ring compounds according to the Examples may increase generation efficiency of excitons in the emission layer. Additionally, the condensed ring compounds according to the Examples had high glass transition temperatures, and deterioration of the condensed ring compounds during a high-temperature process can be effectively prevented.

### Evaluation Example 2. Performance Evaluation of Light-Emitting Device

Properties of the light-emitting devices fabricated as described above were measured at a current density of 10 mA/cm² using V7000 OLED IVL Test System, (Polaronix).

Specifically, a driving voltage (V) at a luminance of 1000 cd/m² was measured using a source meter (Keithley Instrument, 2400 series), and a luminous efficacy (Cd/A) was measured using a luminance meter CS-2000 (Konica Minolta).

The light-emitting device was continuously driven at a current density of 10 mA/cm², and a time until a luminance dropped to 95% of an initial value was measured. A relative value with respect to a measured value from the light-emitting device using the compound of Comparative Example 1 was expressed as a driving voltage, an efficiency, and a life-span of each light-emitting device.

The results are shown in Table 4.

TABLE 4

| | | Relative | Relative | Relative |
|---|---|---|---|---|
| | Compound | driving voltage | efficiency | life-span |
| Example 1 | 10 | 77.1 | 100.1 | 117.9 |
| Example 2 | 8 | 65.6 | 100.0 | 122.2 |
| Example 3 | 13 | 65.5 | 100.0 | 122.2 |
| Example 4 | 16 | 65.5 | 100.0 | 122.2 |
| Example 5 | 17 | 66.9 | 100.0 | 121.8 |
| Comparative | 101 | 100 | 100 | 100 |
| Example 1 | | | | |
| Comparative | 102 | 81.1 | 100.1 | 115.5 |
| Example 2 | | | | |
| Comparative | 103 | 128.7 | 94.9 | 97.8 |
| Example 3 | | | | |
| Comparative | 104 | 115.0 | 90.2 | 101.1 |
| Example 4 | | | | |
| Comparative | 105 | 105.3 | 86.6 | 107.6 |
| Example 5 | | | | |
| Comparative | 106 | 118.9 | 96.1 | 102.7 |
| Example 6 | | | | |

Referring to Table 4, in light-emitting device using the condensed ring compound according to the Examples, the driving voltage was reduced and the relative life-span was improved without degrading the luminous efficiency.

In the light-emitting device using the condensed ring compound according to Comparative Examples, the driving voltage was high and the relative life-span was low.

Although the example embodiments of the present disclosure have been described, it is understood that the present disclosure should not be limited to these example embodiments but various changes and modifications can be made by one ordinary skilled in the art within the spirit and scope of the present disclosure, as set forth in the following claims and equivalents thereof.

What is claimed is:

1. A condensed ring compound represented by Chemical Formula 1: 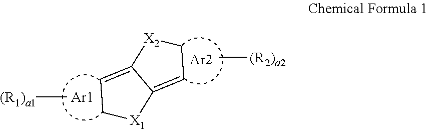
wherein, in Chemical Formula 1,
X₁ and X₂ are each independently oxygen, sulfur, or selenium,
Ar₁ and Ar₂ are each independently a fused C₆-C₆₀ ring comprising an aromatic ring,
R₁ and R₂ are each independently hydrogen, deuterium, —OH, —CN, —F, —Cl, —Br, —I, —SF₅, —CD₃, —CD₂H, —CDH₂, —CF₃, —CF₂H, —CFH₂, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₁-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₁-C₆₀ heteroaryloxy group, a substituted or unsubstituted C₁-C₆₀ heteroarylthio group, a substituted or unsubstituted C₈-C₆₀ condensed polycyclic group, or a substituted or unsubstituted silyl group,
at least one of R₁ and R₂ is an electron-withdrawing group or a group substituted with an electron-withdrawing group, wherein the electron-withdrawing group has a para Hammett substituent constant (σₚ) of greater than 0 according to the Hammett equation,
a1 and a2 are each independently an integer of 1 to 6,
when R₁ and R₂ are each independently 2 or more, two or more of each of R₁ and R₂ are the same or different,
two or more of adjacent R₁ are optionally combined with each other to form a saturated ring or an unsaturated ring, and
two or more of adjacent R₂ are optionally combined with each other to form a saturated ring or an unsaturated ring.

2. The condensed ring compound of claim 1, wherein the electron-withdrawing group is each independently —F, —Cl, —Br, —I, —SF₅, —CF₃, —CN, —SCN, —SOCH₃, —SOCH₂CH₃, —SCH(CH₃)₂, —NO₂, or a π electron-depleted nitrogen-containing C₃-C₃₀ cyclic group.

3. The condensed ring compound of claim 2, wherein the electron-withdrawing group is each independently —F, —Cl, —Br, —I, —SF₅, —CF₃, —CN, —SCN, —SOCH₃, —SOCH₂CH₃, —SCH(CH₃)₂, —NO₂, a substituted or unsubstituted triazine group, a substituted or unsubstituted thiazole group, a substituted or unsubstituted benzothiazole group, a substituted or unsubstituted pyrazine group, a substituted or unsubstituted pyridine group, a substituted or unsubstituted pyridazine group, a substituted or unsubstituted pyrimidine group, a substituted or unsubstituted naphthyridine group, a substituted or unsubstituted phthalazine group, a substituted or unsubstituted quinoline group, a substituted or unsubstituted quinazoline group, a substituted or unsubstituted benzocinnoline group, a substituted or unsubstituted phenanthroline group, or a substituted or unsubstituted acridine group.

4. The condensed ring compound of claim 3, wherein the substituted triazine group, the substituted thiazole group, the substituted benzothiazole group, the substituted pyrazine group, the substituted pyridine group, the substituted pyridazine group, the substituted pyrimidine group, the substituted naphthyridine group, the substituted phthalazine group, the substituted quinoline group, the substituted quinazoline group, the substituted benzocinnoline group, the substituted phenanthroline group, and the substituted acridine group are each independently substituted with at least one of —F, —Cl, —Br, —I, —SF₅, —CF₃, —CN, —SCN, —SOCH₃, —SOCH₂CH₃, —SCH(CH₃)₂, —NO₂, a triazine group, a thiazole group, a benzothiazole group, a pyrazine group, a pyridine group, a pyridazine group, a pyrimidine group, a naphthyridine group, a phthalazine group, a quinoline group, a quinazoline group, a benzocinnoline group, a phenanthroline group, or an acridine group.

5. The condensed ring compound of claim 1, wherein Ar₁ and Ar₂ are each independently a group in which a benzene ring, a naphthalene ring, an anthracene ring, a phenanthrene ring, a tetracene ring, a pyrene ring, or a benzopyrene ring is fused.

6. The condensed ring compound of claim 1, wherein the polycyclic compound is represented by Chemical Formula 1-1: 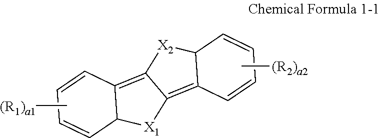
wherein, in Chemical Formula 1-1,
a1 and a2 are each independently an integer of 1 to 4.

7. The condensed ring compound of claim 1, wherein the condensed ring compound is represented by Chemical Formula 1-2, Chemical Formula 1-3, or Chemical Formula 1-4: 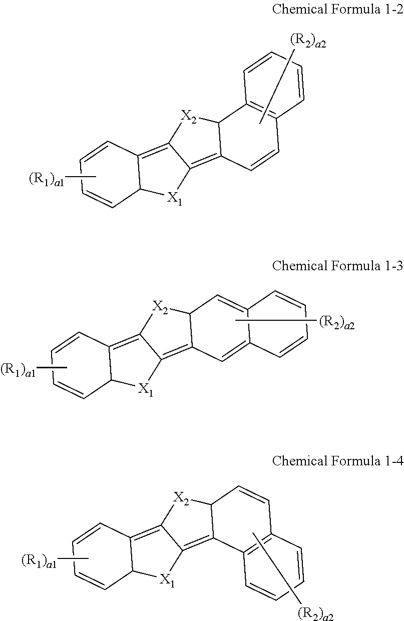
wherein, in Chemical Formulae 1-2, 1-3, and 1-4,
a1 is an integer of 1 to 4, and
a2 is an integer of 1 to 6.

8. The condensed ring compound of claim 1, wherein the condensed ring compound is represented by Chemical Formula 1-5, Chemical Formula 1-6, Chemical Formula 1-7, Chemical Formula 1-8, Chemical Formula 1-9, or Chemical Formula 1-10:

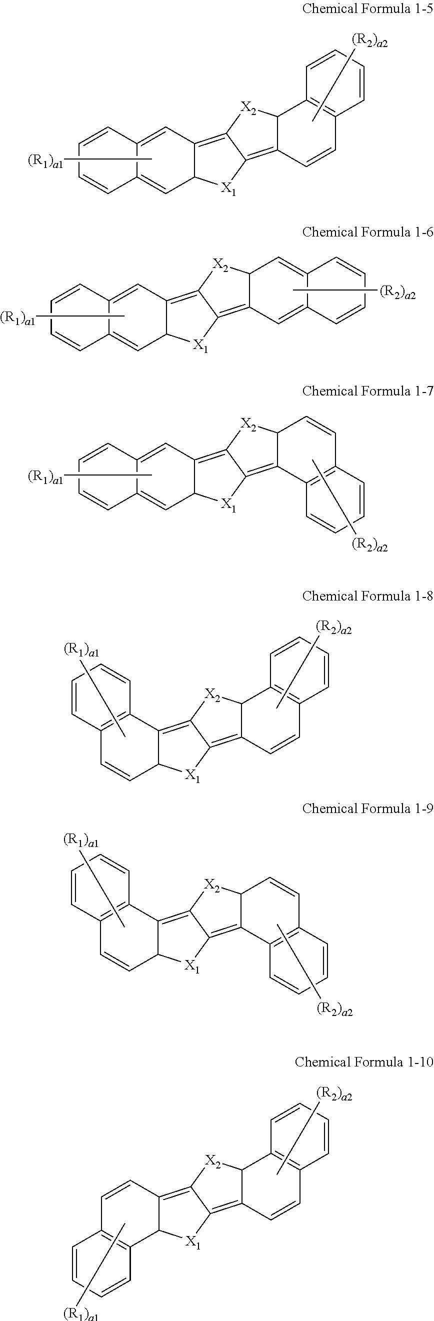

9. The condensed ring compound of claim 1, wherein the condensed ring compound has a hole mobility from 1.30×10⁻³ centimeter squared per volt second to 2.50×10⁻² centimeter squared per volt second, as determined by a space-charge-limited current (SCLC) method.

10. The condensed ring compound of claim 1, wherein the condensed ring compound has an electron mobility from 4.00×10⁻³ centimeter squared per volt second to 3.00×10⁻² centimeter squared per volt second, as determined by a space-charge-limited current (SCLC) method.

11. The condensed ring compound of claim 1, wherein the condensed ring compound has a glass transition temperature from 110° C. to 190° C., as determined by differential scanning calorimetry (DSC).

12. The condensed ring compound of claim 1, wherein the condensed ring compound has a HOMO energy level from −9.0 electron volts to −6.0 electron volts, as determined by cyclic voltammetry (CV).

13. The condensed ring compound of claim 1, wherein the condensed ring compound has a LUMO energy level from −5.8 electron volts to −4.9 electron volts, as determined by cyclic voltammetry (CV).

14. The condensed ring compound of claim 1, wherein the condensed ring compound is one of compounds 1 to 36:

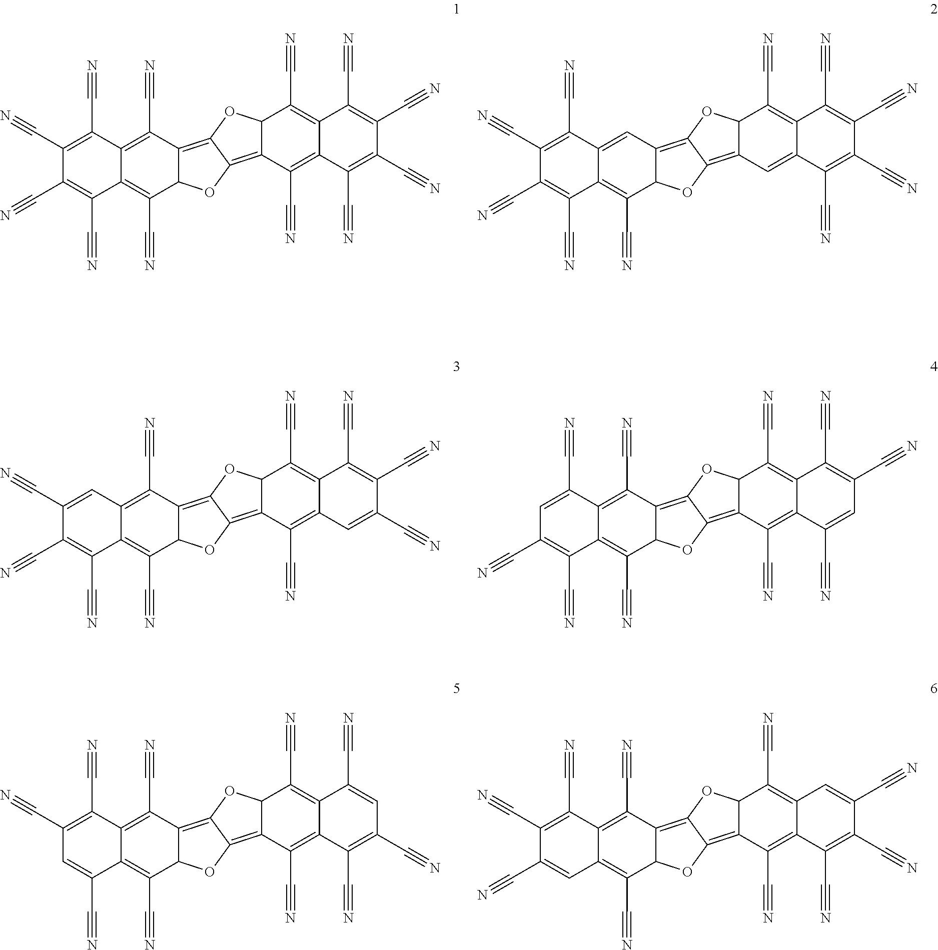

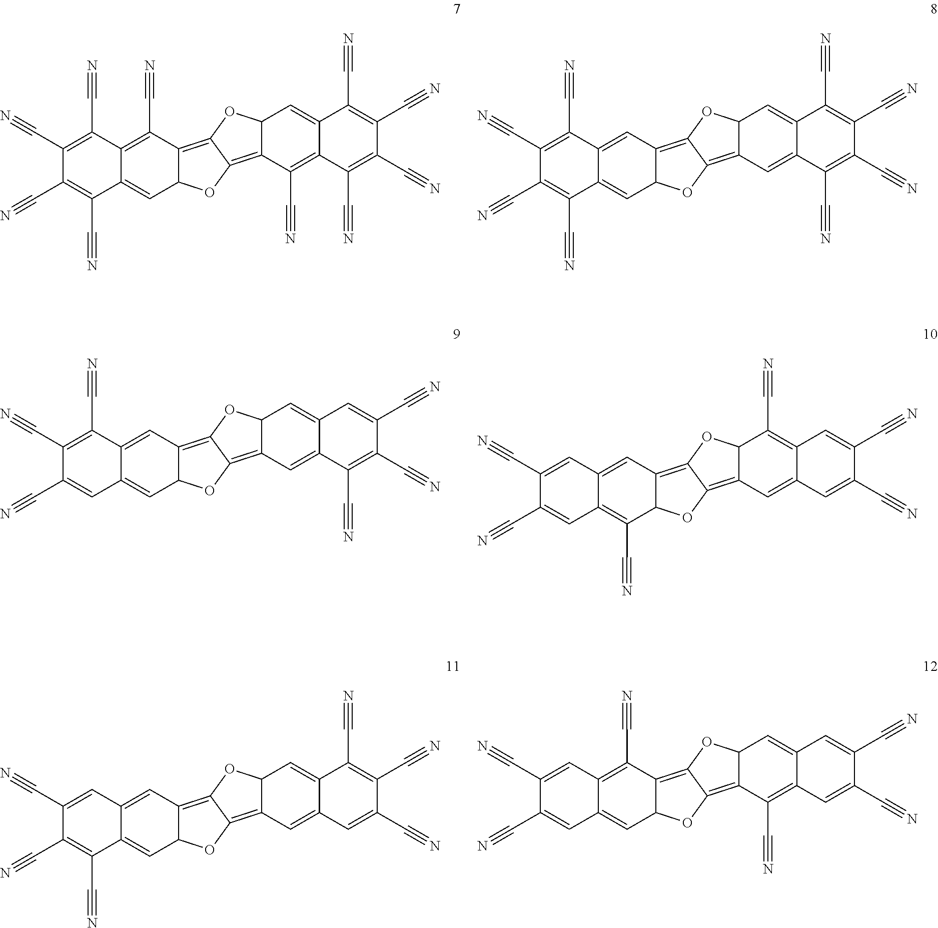 [SMILES: N#CC1=c2c(C#N)c(C#N)c(C#N)c(C#N)c2=CC2OC3=C4C(C#N)=c5c(C#N)c(C#N)c(C#N)c(C#N)c5=CC4OC3=C12 ; N#Cc1c(C#N)c(C#N)c2c(c1C#N)=CC1=C3OC4C=c5c(C#N)c(C#N)c(C#N)c(C#N)c5=CC4=C3OC1C=2 ; N#Cc1cc2c(c(C#N)c1C#N)=CC1=C3OC4C=c5cc(C#N)c(C#N)c(C#N)c5=CC4=C3OC1C=2 ; N#CC1=c2cc(C#N)c(C#N)cc2=CC2=C3OC4C(=C3OC21)C=c1cc(C#N)c(C#N)cc1=C4C#N ; N#Cc1cc2c(c(C#N)c1C#N)=CC1OC3=C4C=c5cc(C#N)c(C#N)c(C#N)c5=CC4OC3=C1C=2 ; N#CC1=c2cc(C#N)c(C#N)cc2=CC2OC3=C4C(C#N)=c5cc(C#N)c(C#N)cc5=CC4OC3=C12]

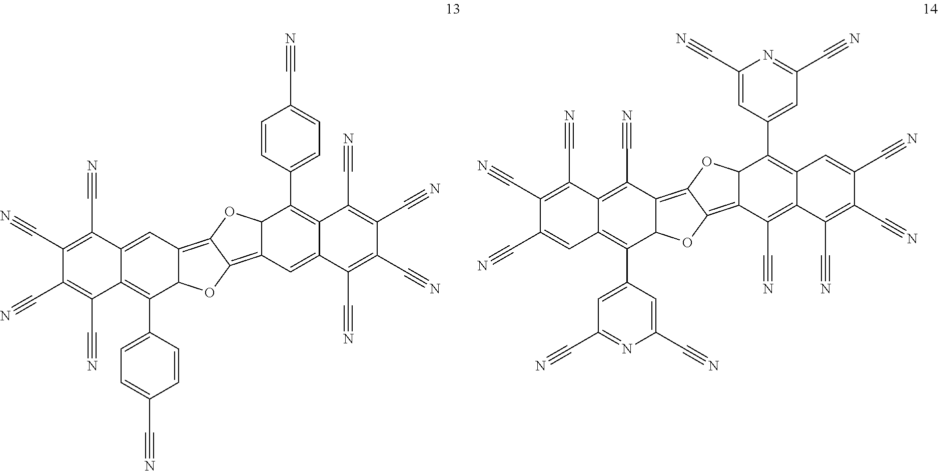 [SMILES: N#Cc1ccc(C2=c3c(C#N)c(C#N)c(C#N)c(C#N)c3=CC3=C4OC5C(=C4OC32)C=c2c(C#N)c(C#N)c(C#N)c(C#N)c2=C5c2ccc(C#N)cc2)cc1 ; N#CC1=c2c(C#N)c(C#N)c(C#N)cc2=C(c2cc(C#N)nc(C#N)c2)C2OC3=C4C(C#N)=c5c(C#N)c(C#N)c(C#N)cc5=C(c5cc(C#N)nc(C#N)c5)C4OC3=C12]

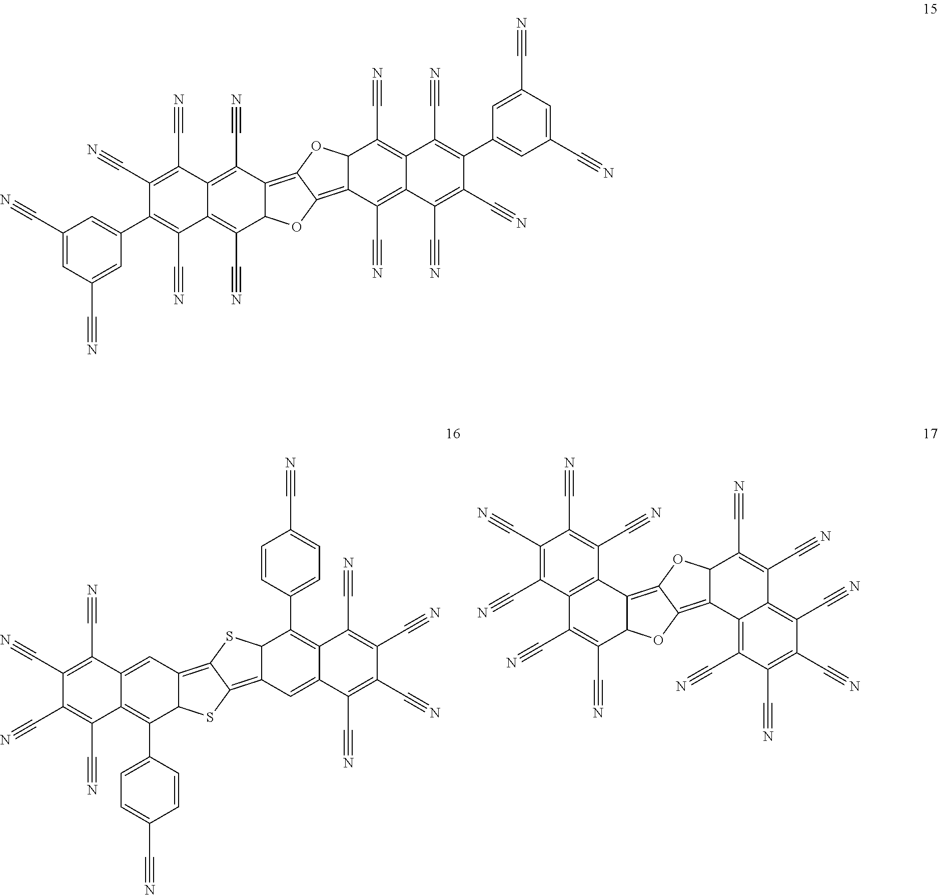 [SMILES: N#CC1=c2c(C#N)c(C#N)c(-c3cc(C#N)cc(C#N)c3)c(C#N)c2=C(C#N)C2OC3=C4C(C#N)=c5c(C#N)c(C#N)c(-c6cc(C#N)cc(C#N)c6)c(C#N)c5=C(C#N)C4OC3=C12 ; N#Cc1ccc(C2=c3c(C#N)c(C#N)c(C#N)c(C#N)c3=CC3=C4SC5C(=C4SC32)C=c2c(C#N)c(C#N)c(C#N)c(C#N)c2=C5c2ccc(C#N)cc2)cc1 ; N#CC1=C(C#N)C2OC3=C4c5c(C#N)c(C#N)c(C#N)c(C#N)c5C(C#N)=C(C#N)C4OC3=C2c2c(C#N)c(C#N)c(C#N)c(C#N)c21]

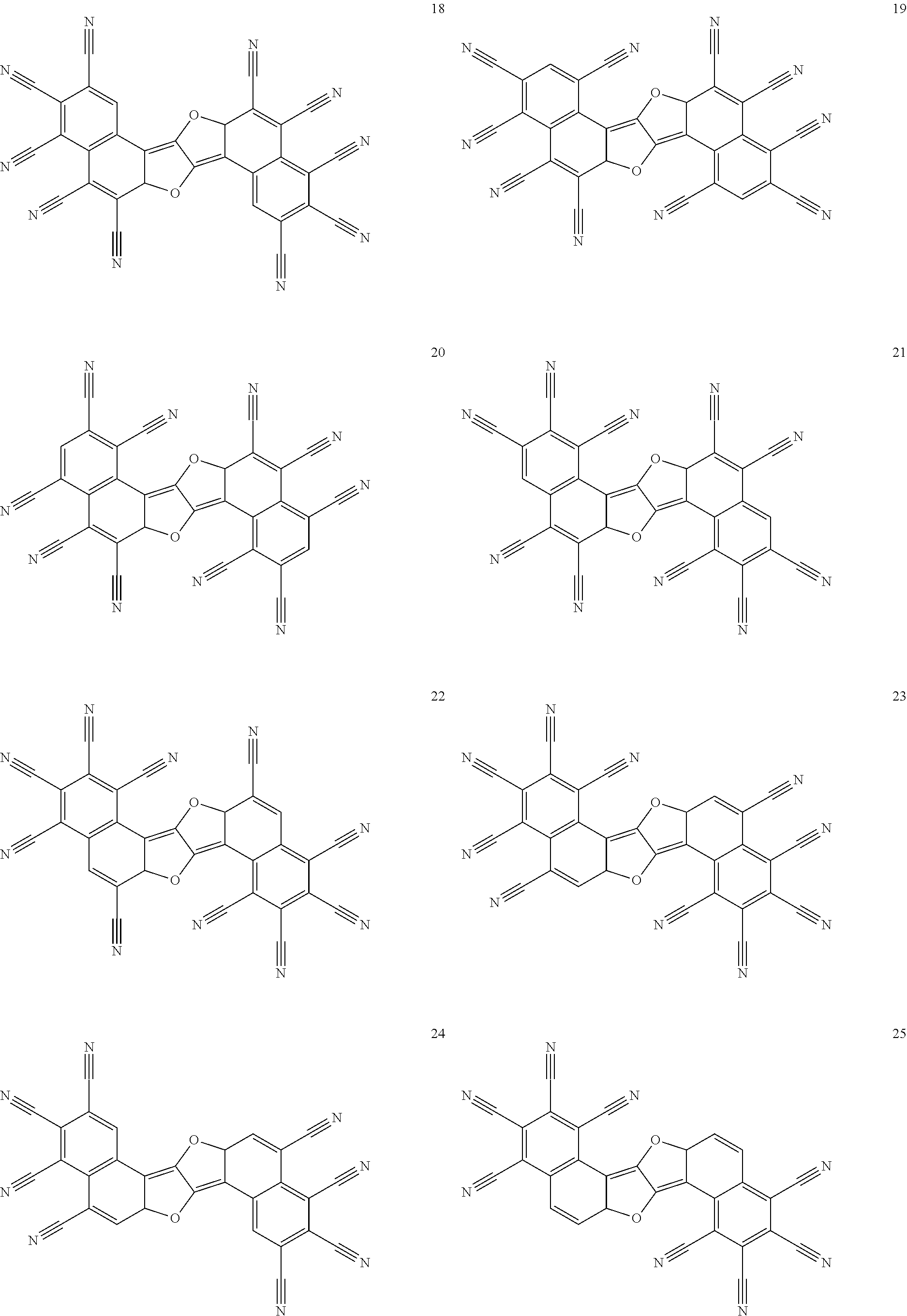

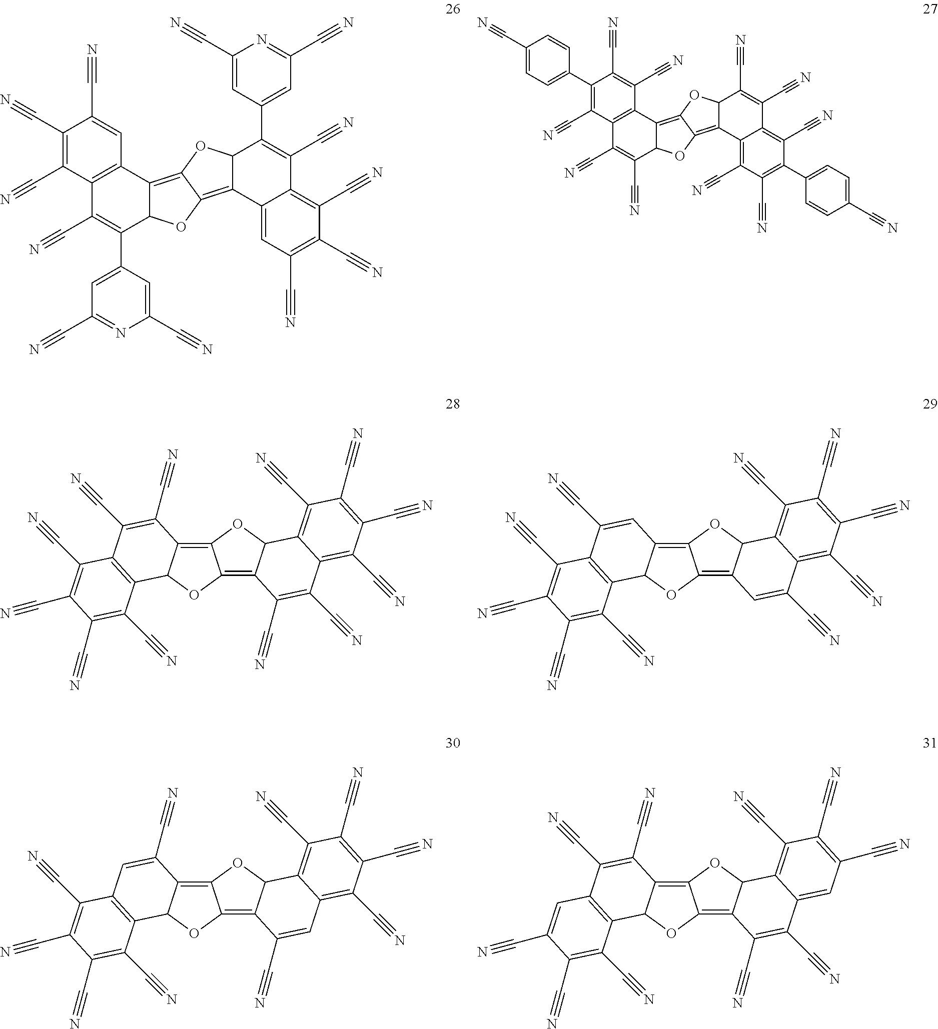

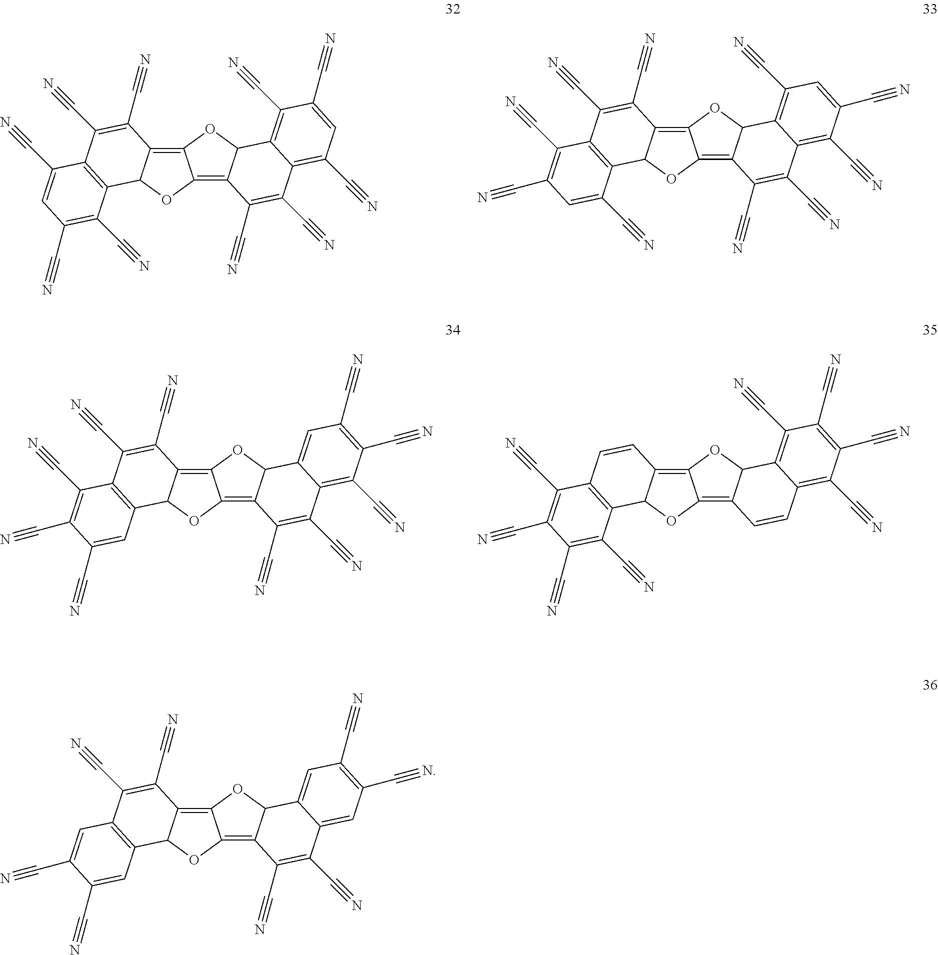 [SMILES: N#CC1=C(C#N)c2c(C#N)cc(C#N)c(C#N)c2C2OC3=C4C(C#N)=C(C#N)c5c(C#N)cc(C#N)c(C#N)c5C4OC3=C12 ; N#CC1=C(C#N)c2c(C#N)c(C#N)cc(C#N)c2C2OC3=C4C(C#N)=C(C#N)c5c(C#N)c(C#N)cc(C#N)c5C4OC3=C12 ; N#CC1=C(C#N)c2c(cc(C#N)c(C#N)c2C#N)C2OC3=C4C(C#N)=C(C#N)c5c(cc(C#N)c(C#N)c5C#N)C4OC3=C12 ; N#Cc1c(C#N)c(C#N)c2c(c1C#N)C=CC1=C3OC4C(=C3OC12)C=Cc1c(C#N)c(C#N)c(C#N)c(C#N)c14 ; N#CC1=C(C#N)c2cc(C#N)c(C#N)cc2C2OC3=C4C(C#N)=C(C#N)c5cc(C#N)c(C#N)cc5C4OC3=C12]

15. A light-emitting device, comprising:
a first electrode;
a second electrode; and
an intermediate layer disposed between the first electrode and the second electrode,
wherein the intermediate layer comprises a hole transfer region, an emission layer, and an electron transfer region, and the hole transfer region comprises a condensed ring compound represented by Chemical Formula 1: 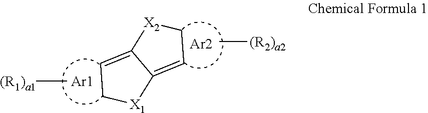
wherein, in Chemical Formula 1,
X₁ and X₂ are each independently oxygen, sulfur, or selenium,
Ar₁ and Ar₂ are each independently a fused C₆-C₆₀ ring comprising an aromatic ring,
R₁ and R₂ are each independently hydrogen, deuterium, —OH, —CN, —F, —Cl, —Br, —I, —SF₅, —CD₃, —CD₂H, —CDH₂, —CF₃, —CF₂H, —CFH₂, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₁-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₁-C₆₀ heteroaryloxy group, a substituted or unsubstituted C₁-C₆₀ heteroarylthio group, a substituted or unsubstituted C₈-C₆₀ condensed polycyclic group, or a substituted or unsubstituted silyl group,
at least one of R₁ and R₂ is an electron-withdrawing group or a group substituted with an electron-withdrawing group, wherein the electron-withdrawing group has a para Hammett substituent constant (σₚ) of greater than 0 according to the Hammett equation,
a1 and a2 are each independently be an integer of 1 to 6,
when R₁ and R₂ are each independently 2 or more, two or more of each of R₁ and R₂ are the same or different,
two or more adjacent R₁ are optionally combined with each other to form a saturated ring or an unsaturated ring, and
two or more of adjacent R₂ are optionally combined with each other to form a saturated ring or an unsaturated ring.

16. The light-emitting device of claim 15, wherein the hole transfer region comprises at least one of a hole injection layer, a hole transport layer, or an electron blocking layer, and at least one of the hole injection layer, the hole transport layer, or the electron blocking layer comprises the condensed ring compound.

17. The light-emitting device of claim 16, wherein at least one of the hole injection layer, the hole transport layer, or the electron blocking layer comprises one or more of the condensed ring compound.

18. The light-emitting device of claim 15, wherein the emission layer emits a blue light having a maximum emission central wavelength in a range from 430 nanometers to 490 nanometers.

19. An electronic device, comprising the light-emitting device of claim 15.

20. The electronic device of claim 19, wherein the electronic device is one of a flat panel display, a curved display, a computer monitor, a medical monitor, a television, a billboard, a light for indoor or outdoor lighting, a signal lighting, a head-up display, a full or partial transparent display, a flexible display, a rollable display, a foldable display, a stretchable display, a laser printer, a phone, a mobile phone, a tablet, a phablet, a personal information terminal (PDA), a wearable device, a laptop computer, a digital camera, a camcorder, a viewfinder, a micro display, a 3D display, a virtual reality or augmented reality display, a vehicle, a video wall including multiple displays tiled together, a theater or stadium screen, a phototherapy device, or a signage.
